(19) United States
(12) Patent Application Publication
(10) Pub. No.: US 2026/0022376 A1
(43) Pub. Date: Jan. 22, 2026

(54) TAU-SEED INTERACTOR INHIBTORS FOR THE TREATMENT OF NEURODEGENERATIVE DISORDERS

(71) Applicant: The Trustees of Indiana University, Bloomington, IN (US)

(72) Inventor: Cristian Lasagna-Reeves, Indianapolis, IN (US)

(21) Appl. No.: 18/998,412

(22) PCT Filed: Jul. 24, 2023
(86) PCT No.: PCT/US2023/070854

§ 371 Date: Jan. 24, 2025

Related U.S. Application Data
(60) Provisional application No. 63/391,829, filed on Jul. 25, 2022.

(51) Int. Cl. C12N 15/113; A61P 25/28; C12N 15/86

(52) U.S. Cl. CPC C12N 15/113; A61P 25/28; C12N 15/86; C12N 2310/14; C12N 2310/531; C12N 2750/14143

(57) ABSTRACT

The present disclosure provides novel approaches to the treatment of Alzheimer's disease, and other neurodegenerative disorders such as chronic traumatic encephalopathy (CTE) using novel therapeutics comprising agents that reduce the interaction of a tau seed interactor with intracellular tau proteins and thus reduce or inhibit the production of tau-associated neurofibrillary tangles.

### CROSS-REFERENCE TO RELATED APPLICATIONS

This application is a U.S. National Phase Entry of PCT Patent Application PCT/US2023/070854, filed Jul. 24, 2023, which claims the benefit of priority to U.S. Provisional Patent Application 63/391,829, filed Jul. 25, 2022, the entire contents of each are incorporated herein by reference.

### REFERENCE TO GOVERNMENT GRANTS

This invention was made with government support under NS119280 awarded by the National Institutes of Health. The Government has certain rights in the invention.

### INCORPORATION BY REFERENCE OF A SEQUENCE LISTING XML

A Sequence Listing is provided herewith as a Sequence Listing xml titled “IUIC-00089 (2022-22-02).xml” created on Jul. 17, 2023, and having a size of 10,283 bytes. The entire contents of the Sequence Listing xml are incorporated herein by reference.

### FIELD OF THE INVENTION

The general field of the present disclosure are novel approaches to the treatment of Alzheimer's disease, and other neurodegenerative disorders such as chronic traumatic encephalopathy (CTE) using novel therapeutics comprising agents that reduce the interaction of a tau seed interactor with intracellular tau proteins and thus reduce or inhibit the production of tau-associated neurofibrillary tangles.

### BACKGROUND

Pathological aggregation of tau and the preponderance of neurofibrillary tangles (NFT) and other tau inclusions are defining histopathological features of Alzheimer's disease (AD), Pick's disease (PiD), progressive supranuclear palsy (PSP), and many neurodegenerative diseases collectively known as tauopathies. See Alonso et al., “Mechanism of tau-induced neurodegeneration in Alzheimer disease and related tauopathies,” (2008) Curr Alzheimer Res 5: pp. 375-384; Lee et al., “Neurodegenerative tauopathies, (2001) Annu Rev Neurosci 24: pp. 1121-1159. A major focus of research has been understanding the propagation of pathological tau along neuronal networks in the brains of AD patients. Despite the knowledge acquired, the cellular mechanisms involved in tau propagation remain unclear, though reports have linked it to synaptic activity. See Pooler et al., “Physiological release of endogenous tau is stimulated by neuronal activity,” (2013) EMBO Rep 14: pp. 389-394; Yamada et al., “Neuronal activity regulates extracellular tau in vivo,” (2014) J Exp Med 211: pp. 387-393. It has been recently shown that increases in neuronal activity stimulate the release of tau in vitro and enhance tau pathology in vivo. See Wu et al., “Neuronal activity enhances tau propagation and tau pathology in vivo,” (2016) Nat Neurosci 19: pp. 1085-1092. Tau released due to neuronal activity could be impaired by blocking presynaptic vesicle release, suggesting that tau is released via presynaptic compartments. See Pooler et al. 2013. Furthermore, seed-competent tau is significantly enriched in AD patient synapses, suggesting that tau seeds propagate through the brain along synaptically connected neurons. See DeVos et al., “Synaptic Tau Seeding Precedes Tau Pathology in Human Alzheimer's Disease Brain,” (2018) Front Neurosci 12: p. 267. Unfortunately, the nature of the tau species involved in tau spreading and the precise seeding mechanism and template remain unclear. Despite this uncertainty, some studies suggest that a rare species of soluble phosphorylated high molecular weight (HMW) tau is involved in trans-synaptic propagation. See Takeda et al., “Neuronal uptake and propagation of a rare phosphorylated high-molecular-weight tau derived from Alzheimer's disease brain,” (2015) Nat Commun 6: p. 8490; Tanaka et al., “Seeding Activity-Based Detection Uncovers the Different Release Mechanisms of Seed-Competent Tau Versus Inert Tau via Lysosomal Exocytosis,” (2019) Front Neurosci 13: p. 1258. Whether these HMW tau-containing particles are exclusively comprised of tau or contain other constituents, such as proteins or lipids for propagation, is unknown.

Numerous studies on the tau interactome have established that tau interacts directly with proteins and complexes involved in various biological functions in addition to those associated with microtubule stability. See Eftekharzadeh et al., “Tau Protein Disrupts Nucleocytoplasmic Transport in Alzheimer's Disease,” (2018) Neuron 99: pp. 925-940; Ittner et al., “Phosphorylated Tau interacts with c-Jun N-terminal kinase-interacting protein 1 (JIP1) in Alzheimer disease,” (2009) J Biol Chem 284: pp. 20909-20916; Ittner et al., “Dendritic function of tau mediates amyloid-beta toxicity in Alzheimer's disease mouse models,” (2010) Cell 142: pp. 387-397; McInnes et al., “Synaptogyrin-3 Mediates Presynaptic Dysfunction Induced by Tau, (2018) Neuron 97: pp. 823-835; Sohn et al., “Pathogenic Tau Impairs Axon Initial Segment Plasticity and Excitability Homeostasis,” (2019) Neuron 104: pp. 458-470; Vanderweyde et al., “Interaction of tau with the RNA-Binding Protein TIA1 Regulates tau Pathophysiology and Toxicity,” (2016) Cell Rep 15: pp. 1455-1466; Morris et al., “The many faces of tau,” (2011) Neuron 70: pp. 410-426. Mass spectrometry studies have identified interactors of total tau in vivo in tauopathy mouse model. See Liu et al., “Co-immunoprecipitation with Tau Isoform-specific Antibodies Reveals Distinct Protein Interactions and Highlights a Putative Role for 2N Tau in Disease,” (2016) J Biol Chem 291: pp. 8173-8188; Maziuk et al., “RNA binding proteins co-localize with small tau inclusions in tauopathy,” (2018) Acta Neuropathol Commun 6: p. 71; Choi et al., “Acetylation changes tau interactome to degrade tau in Alzheimer's disease animal and organoid models,” (2020) Aging Cell 19: e13081; Wang et al., “Tau interactome mapping based identification of Otub1 as Tau deubiquitinase involved in accumulation of pathological Tau forms in vitro and in vivo,” (2017) Acta Neuropathol 133: pp. 731-749. Other studies have examined the interactome of total tau in SH-SY5Y neuroblastoma cells, a mixed population of neuroprogenitor-derived human ReN cells, and iPSC-derived neurons. See Gunawardana et al., “The Human Tau Interactome: Binding to the Ribonucleoproteome, and Impaired Binding of the Proline-to-Leucine Mutant at Position 301 (P301L) to Chaperones and the Proteasome,” (2015) Mol Cell Proteomics 14: pp. 3000-3014; Wang et al., “Tau interactome analyses in CRISPR-Cas9 engineered neuronal cells reveal ATPase-dependent binding of wild-type but not P301L Tau to non-muscle myosins,” (2019) Sci Rep 9: p. 16238; Tracy et al., “Tau interactome maps synaptic and mitochondrial processes associated with neurodegeneration,” (2022) Cell 185: pp. 712-728. However, no studies have directly compared the interactomes of seeding-competent tau aggregates with those of monomeric tau or determined how tau-seed interactors affect the nature of this seed and, subsequently, tau propagation.

However, what is needed is an understanding as to how tau spreading that the role of interactors of tau seeds can be utilized in treating and preventing tauopathies including Alzheimer's disease and chronic traumatic encephalopathy (CTE). The present invention addresses this need.

### SUMMARY OF THE INVENTION

Pathological tau aggregation is a defining histopathological feature of Alzheimer's disease (AD) and other neurodegenerative diseases collectively known as tauopathies. The propagation of pathological forms of tau in AD patient brains has been shown to follow neuronal networks. However, the cellular mechanisms involved in tau propagation and the nature of the tau species involved in spreading remain unclear. The inventors have identified the proteins that specifically interact with this tau seed.

The current invention discloses the identification of bassoon (BSN), a scaffolding presynaptic protein, as an interactor of the tau seed isolated from a mouse model of tauopathy, and from AD and progressive supranuclear palsy (PSP) postmortem samples. It is shown that BSN exacerbates tau seeding and toxicity in vivo and that BSN downregulation significantly decreases tau spreading and overall disease pathology in vivo, rescuing synaptic and behavioral impairment and ameliorating brain atrophy.

The invention provides methods using interactors, including BSN, to stabilize tau seeds as a therapeutic approach for neurodegenerative tauopathies including Alzheimer's disease and related disorders including chronic traumatic encephalopathy (CTE).

More specifically, the current invention provides methods of treating neurodegenerative tauopathies in a patient in need thereof comprising administration of an agent that reduces or inhibits the interaction of a tau seed interactor with intracellular tau proteins.

In some embodiments, the tau seed interactor is Bassoon (BSN), a scaffolding protein of the presynaptic active zone. In some embodiments, the agent comprises an adeno-associated virus (AAV)-containing an a short-hairpin RNA (shRNA) against BSN (shBSN).

In some embodiments, the shBSN sequence is CCTAACGCTTTCCTCTGACAT (SEQ ID NO: 1).

In other embodiments, the shBSN can be attached to or part of any vector known in the art including plasmids, viral vectors, bacteriophages, cosmids, and artificial chromosomes.

In other embodiments, the agent comprises a monoclonal antibody directed against the tau seed interactor. In yet other embodiments, the agent comprises a monoclonal antibody directed against BSN. In still other embodiments, the agent is an siRNA or antisense oligonucleotide that targets BSN.

In any of the embodiments, the agent that reduces or inhibits the interaction of a tau seed interactor with intracellular tau proteins leads to a reduction of neurodegeneration in a patient with a neurodegenerative tauopathies.

In any of the embodiments, the agent that reduces or inhibits the interaction of a tau seed interactor with intracellular tau proteins leads to a behavioral improvement in a patient with a neurodegenerative tauopathies.

The current invention also provides methods of preventing neurodegenerative tauopathies in a patient in need thereof comprising administration of an agent that reduces or inhibits the interaction of a tau seed interactor with intracellular tau proteins.

In any of the embodiments described herein, the neurodegenerative tauopathies can be neurodegenerative diseases characterized by abnormal metabolism of misfolded tau proteins which in turn leads to intracellular accumulation and formation of neurofibrillary tangles (NFT). In any embodiment, the neurodegenerative tauopathies are selected from Alzheimer's disease, progressive supranuclear palsy (PSP), frontotemporal lobar degeneration (FTLD-TAU), corticobasal degeneration, Pick's disease (frontal temporal dementia), chronic traumatic encephalopathy (CTE), and primary age related taupathy.

The current invention also provides methods of treating or preventing Alzheimer's disease in a patient in need thereof comprising administration of an agent that reduces that reduces the interaction of a tau seed interactor with intracellular tau proteins.

In yet other embodiments, current invention provides methods of treating or preventing chronic traumatic encephalopathy (CTE) in a patient in need thereof comprising administration of an agent that reduces the interaction of a tau seed interactor with intracellular tau proteins.

The current invention provides pharmaceutical compositions comprising an agent that reduces the interaction of a tau seed interactor with intracellular tau proteins. The pharmaceutical compositions of the current invention can further comprise one or more pharmaceutically acceptable carriers, diluents or excipients.

In any of the methods of the current invention, can further comprise the administration of one or more additional therapeutic agents.

These and other embodiments and features of the disclosure will become more apparent through reference to the following description, the accompanying figures, and the claims. Furthermore, it is to be understood that the features of the various embodiments described herein are not mutually exclusive and can exist in various combinations and permutations.

### BRIEF DESCRIPTION OF THE DRAWINGS

FIG. 1A-H depicts HMW tau seed interacting with BSN protein. FIG. 1A is a schematic of the tau-seeding assay of SEC fractions. FIG. 1B shows tau-seeding activity of SEC fractions obtained from 3-month-old PS19 and wild-type (WT) mouse brain lysates. FIG. 1C: total human tau detected by ELISA in SEC fractions from PS19 and WT mouse brain lysates. FIG. 1D: tau seeding activity of the PS19 SEC fraction 9 (F9) containing HMW tau, before and after hTau immunoprecipitation (IP) using HT7 antibody. FIG. 1E: Electron microscopy (EM) of the F9 IP product showing tau protofibril structures, and width distribution of protofibrils. Scale bar: 100 nm. FIG. 1F-G, Volcano plot indicating tau interactors found in F9 (FIG. 1F) and F17 (FIG. 1G) identified by LC-MS/MS. FIG. 1H: Western blot of BSN from co-immunoprecipitation (co-IP) of human tau (HT7) from F9 in PS19 brain lysates. Colocalization analysis was performed to determine pixel intensity correlation between PHF1 and BSN. Scale bar: 25 μm. Data are shown as the mean±s.e.m. Experiments were performed in triplicates (FIG. 1B-D, H), and significance was determined by unpaired Student's t-test (FIG. 1D).

FIG. 2A-I shows that BSN is associated with tau pathology in human AD and PSP cases. FIG. 2A and FIG. 2B: Tau seeding activity of SEC fractions from human AD lysates (from middle frontal gyrus, MFG) (FIG. 2A), and PSP lysates (from pons) (FIG. 2B), compared to healthy controls from MFG and pons, respectively. FIG. 2C, FIG. 2D: Total human tau detected by ELISA in SEC fractions from human AD (FIG. 2C) and PSP (FIG. 2D) brain lysates compared to healthy controls. FIG. 2E: Tau seeding activity of the AD and PSP SEC fraction 9 (F9) containing HMW tau, before and after hTau immunoprecipitation (IP) using HT7 antibody. FIG. 2F, FIG. 2G: Tau twisted filaments present in the tau-IP product from F9 of AD and PSP SEC fractions, visualized by electron microscopy. Scale bars: 100 nm. (FIG. 2F), and width distribution (FIG. 2G). No filaments were detected in MFG and pons healthy controls. FIG. 2H, Co-IP of human tau (HT7) and BSN from SEC F9 in AD, PSP, control MFG and control pons brain lysates. FIG. 2I: Colocalization between BSN (red) and pathological phosphorylated tau species (PHF1, green) in AD and PSP brain section. Merge panel includes orthogonal image of reconstructed three-dimensional views. Colocalization analysis was performed to determine pixel intensity correlation between PHF1 and BSN. Scale bar: 10 μm. Data are shown as the mean±s.e.m. Experiments were performed with n=3 (FIG. 2A-D, F, H, I) and n=6 (FIG. 2E). significance was determined by one-way ANOVA (FIG. 2E).

FIG. 3A-I depicts the BSN overexpression increases tau-seeding and toxicity by direct interaction. FIG. 3A: Seeding activity of HEK cell lysates expressing hTauP^{301S}, BSN^{WT}, or both plasmids. FIG. 3B: Western blot and quantification of misfolded tau levels detected with MC1 antibody in HEK cells overexpressing hTau^{P301S}, BSN^{WT} or both. FIG. 3C: Representative image of double immunofluorescences between MC1 and BSN in HEK cells overexpressing hTauP^{301S} and BSN^{WT}. Merge panel includes orthogonal image of reconstructed three-dimensional views. Colocalization analysis was performed to determine pixel intensity correlation between MC1 and BSN. Scale bar: 50 μm. FIG. 3D: Co-immunoprecipitation of hTau (HT7 antibody) and immunoblot for BSN in HEK cells overexpressing hTauP^{301S} and BSN^{WT}. FIG. 3E: PLA fluorescence paring of hTau and BSN antibodies, and quantification in HEK cells overexpressing Tau^{WT}, Tau^{WT}/BSN^{WT}, Tau^{P301S} and Ta^{P301S}/BSN^{WT}. FIG. 3F: PLA fluorescence paring of hTau and his-tag antibodies, and quantification in HEK cells overexpressing Tau^{P301S} and Tau^{P301S} plus the N- or C-terminal fragments of BSN (BSN-N or BSN-C, respectively). Scale bar: 50 μm. FIG. 3G-I: Representative images (FIG. 3G), quantification of phenotypic eye degeneration (FIG. 3H), and tau-seeding activity (each point is a pool of 20 fly heads from a distinct eclosion event) (FIG. 3I) in control, BSN^{WT}, BSN^{mut}, hTau^{P301L}, hTau^{P301L}/BSN^{WT} and hTau^{P301L}/BSN^{mut} flies. Data are the mean±s.e.m. Experiments were performed with n=8 (FIG. 3A-C), n=3 (FIG. 3D), n=5 (FIG. 3E, F), n=20 (FIG. 3G, H) and n=6 (FIG. 3I). Significance was determined by one-way ANOVA (FIG. 3A, B, E, F, H, I).

FIG. 4A-D shows that BSN contributes to tau-spreading in vivo. FIG. 4A: Schematic of the GFP-P2A-hTau^{P301L} tau-spreading mouse model. GFP-positive neurons are hTau-donors, spreading hTau^{P301L} to recipient GFP-negative neurons. FIG. 4B: Timeline of injections in wild-type mice. These animals were injected with scramble or shBSN AAV at P0, then with AAV-GFP-P2A-hTau^{P301L} at P90. Animals were euthanized at P180 to evaluate hTau spreading. FIG. 4C, Representative immunofluorescences for hTau (HT7 antibody) in animals overexpressing GFP-P2A-hTau^{P301L} injected with scramble shRNA (left) and BSN shRNA (right). Scale bar: 100 μm. FIG. 4D, Quantification of cortical hTau⁺/GFP⁻ cells (top) and GFP⁺ cells (bottom) per mm². Values are given as the means±s.e.m. Experiments were performed with n=4, and significance was determined by unpaired Student t-test.

FIG. 5A-O shows that BSN downregulation reduces tau pathology and tau-seeding stability in vivo. FIG. 5A: Representative images of hippocampal MC1 immunostaining in 4-month-old PS19 mice injected with shBSN and scramble shRNA. Scale bar: 200 μm, and 50 μm for CA1 and CA3 insets. FIG. 5B: Quantification of MC1 immunostaining in shBSN and scramble PS19 mice as percentage of area. FIG. 5C-E: Hippocampal immunofluorescence (FIG. 5C) using specific antibodies against GFAP (red) and IBA1 (green) in WT_{scramble}, WT_{shBSN}, PS19_{scramble} and PS19_{shBSN} mice. Quantification was performed as percentage of area for IBA1 (FIG. 5D) and GFAP (FIG. 5E) positive cells. FIG. 5F-H: Western blot (FIG. 5F) and quantification of specific antibodies against pTauS396/5404 (PHF1) (FIG. 5G) and pTau Thr231 (FIG. 5H) in shBSN and scramble PS19 mouse brain. FIG. 5I, FIG. 5J: Total human tau levels measured by ELISA (FIG. 5I) and tau-seeding activity (FIG. 5J) in shBSN and scrambled shRNA PS19 mouse brain lysates. FIG. 5K, FIG. 5L: Seeding activity (FIG. 5K), and total hTau levels (FIG. 5L) in SEC fractions from shBSN or scramble PS19 mouse brain lysates. FIG. 5M: Western blot of total tau (HT7, top blot) and misfolded tau (MC1, bottom blot) in SEC fractions (fractions 7 to 10, HMW; fractions 14 to 17, LMW) from shBSN and scramble PS19 mouse brain lysates. FIG. 5N: Representative merge images of immunofluorescence of PSD95 (green) and Syn-1 (magenta). Merge panel includes orthogonal image of reconstructed three-dimensional views. Colocalization analysis was performed to determine pixel intensity correlation between PSD95 and Syn-1. FIG. 5O: Colocalization quantification by Pearson's correlation coefficient. Data are the mean±s.e.m. Experiments were performed with n=8 (FIG. 5A-J, N, O), n=6 (FIG. 5K, L), and n=3 (FIG. 5M). Significance was determined by unpaired Student's t-test (FIG. 5B, G-J) and one-way ANOVA (FIG. 5D, E, O).

FIG. 6A-D shows BSN downregulation restores electrophysiological impairments in PS19 mice. FIG. 6A: Superimposed fEPSP traces produced by stimulation of the Schaffer collateral pathway and recorded in CA1 brain slices before (gray dotted lines) and after (solid lines) LTP induction. Vertical bar: 300 mV, Horizontal bar: 10 ms. FIG. 6B-D: Summary of in vivo LTP (FIG. 6B), averaged from minute 60 to 70 (FIG. 6C), and paired-pulse ratio (PPR) (FIG. 6D) in 6-month-old WT and PS19 mice injected with shBSN or scramble shRNA. Data represent the mean±s.e.m. Experiments were performed with n=11 for WT_{scramble}, n=11 for PS19_{scramble}, n=10 for WT_{shBSN}, and n=11 for PS19_{shBSN} mice (FIG. 6B-D). Significance was determined by two-way ANOVA (FIG. 6C, D).

FIG. 7A-J shows that reducing BSN levels improves behavioral deficit and diminishes pathological tau species in a late stage of pathology. FIG. 7A-D, 2-paw (FIG. 7A), 4-paw test (FIG. 7B), body temperature (FIG. 7C) and (FIG. 7D) frailty test in 9-month-old WT and PS19 mice injected with shBSN or scramble shRNA. FIG. 7E: Hippocampal MC1 immunostaining in 9-month-old PS19 mice cohort injected with shBSN and scramble shRNA. Scale bar: 200 μm, and 50 μm for CA1 and CA3 insets. FIG. 7F: Quantification of MC1 immunostaining in shBSN and scramble PS19 mice as percentage of area. FIG. 7G-I: Western blot and (FIG. 7G) quantification of specific antibodies against pTauS396/5404 (PHF1) (FIG. 7H) and pTau Thr231 (FIG. 7I) in shBSN and scramble PS19 mouse brain lysates. FIG. 7J: Total human tau levels by ELISA in shBSN and scrambled PS19 mouse brain lysates. Experiments were performed with n=20 for WT_{scramble}, n=21 for PS19_{scramble}, n=22 for WT_{shBSN}, and n=16 for PS19_{shBSN} mice (FIG. 7A-D), n=8 (FIG. 7E-J). Significance was determined by one-way ANOVA (FIG. 7A-D) and unpaired Student's t-test (FIG. 7F, H-J).

FIG. 8A and FIG. 8B shows that HMW tau seed represents a low percentage of total tau in the PS19 mouse brain. FIG. 8A, Schematic of aggregated tau denaturation by guanidine HCl (Gdn HCl). FIG. 8B, hTau present in F9 (gray bars) as a percentage of total brain lysates (green bars) before and after denaturation with 3M of Gdn-HCl. Total human tau levels were assayed by ELISA. Data represent the mean±s.e.m. Experiment was performed with n=3.

FIG. 9A-D shows BSN is associated with tau pathology in PS19 mice. FIG. 9A: Representative immunofluorescence and colocalization for BSN (red), PHF1 (green), nuclei (cyan), and merge (yellow), in 3-, 6- and 9-month-old wild-type and PS19 mouse cortexes. Scale bar: 200 μm for main images and 50 μm for insets. Merge image for PS19 includes orthogonal image of reconstructed three-dimensional views. Colocalization analysis was performed to determine pixel intensity correlation between PHF1 and BSN. FIG. 9B: Pearson's correlation of BSN and PHF1 intensity in 3-, 6- and 9-month-old wild-type and PS19 mouse cortexes. FIG. 9C: 3-month-old PS19 mouse sections immunostained against PHF1, BSN, and Syn-1 (upper panels), and colocalization of PHF1 and BSN (colored yellow; lower left panel). PHF1/BSN colocalization was re-colocalized with the Syn-1 channel to obtain a final triple-colocalization between PHF1/BSN and Syn-1 (lower middle and right panels). FIG. 9D: Percentage of colocalization between PHF1/BSN puncta and Syn-1. Data are the mean±s.e.m. Experiments were performed with n=3 (FIG. 9A-D). Significance was determined by one-way ANOVA (FIG. 9B).

FIG. 10A and FIG. 10B shows that BSN colocalizes with a pathological tau species in human AD and PSP cases. FIG. 10A, B: Immunofluorescence, merge and colocalization images of BSN (red) and pTauS396/5404 (PHF1, green) in human AD (from middle frontal gyrus, MFG) (FIG. 10A), and PSP (from pons) (FIG. 10B), and healthy control subjects from MFG and pons, respectively. Merge image includes orthogonal image of reconstructed three-dimensional views. Scale bar: 200 μm for main images and 50 μm for insets. Experiments were performed in triplicates for AD and PSP cases.

FIG. 11A-M shows bassoon overexpression increases tau accumulation in vitro and in a Drosophila model of tauopathy. FIG. 11A-C: hTau levels by ELISA (FIG. 11A), Western blot of total tau (FIG. 11B), seeding activity (FIG. 11C) from HEK cells overexpressing hTau^{WT} or hTau^{P301S}. Western blot membrane was overexposed in the upper level for better visualization of oligomeric tau (denoted by the asterisk*). FIG. 11D: Location of BSN peptides detected by IP-mass spec. FIG. 11E: Representative images, and quantification of 6X-His immunofluorescences from HEK cells overexpressing Tau^{P301S} and 6X-His-BSN N- or C-terminal fragments (BSN-N or BSN-C, respectively). FIG. 11F: Disordered residues of human and mouse bassoon. 85.1% of residues in human BSN and 85.8% of residues in mouse BSN were predicted to be disordered. FIG. 11G: Hydrophobicity profile of bassoon using method of Kyte & Doolittle. Average hydrophobicity is −0.81 for human BSN, and −0.85 mouse BSN. FIG. 11H: Western blot confirming the overexpression of the UAS-BSN under the GMR-Gal4 driver in different fly lines. FIG. 11I: Western blot for BSN of co-immunoprecipitation (Co-IP) of human tau (HT7) from hTau^{P301L}/BSN^{WT} and hTau^{P301L}/BSN^{mut} fly head lysates. FIG. 11J, K: Western blot (FIG. 11J) of the level of HMW and 117 kDa MC1 detected and quantified (FIG. 11K) in Tau^{P301L} Tau^{P301L}/BSN^{WT} and Ta^{P301L}/BSN^{mut} fly head lysates. Each sample is a pool of 20 fly brains from 3 different eclosion events. FIG. 11L, M Western blot (FIG. 11L) and quantification (FIG. 11M) of misfolded tau (MC1) in fly lysates denatured by increasing concentrations of guanidine HCl, in hTau^{P301L}, hTau^{P301L}/BSN^{WT} and hTau^{P301L}/BSN^{mut} head lysates. Data are the mean±s.e.m. Experiments were performed with n=3 (FIG. 11A-C, E, J-M). Significance was determined by unpaired Student's t-test (FIG. 11B-E) and one-way ANOVA (FIG. 11K, in).

FIG. 12A-D shows that murine BSN downregulation does not produce gross brain abnormalities. FIG. 12A: Western blot and quantification of BSN downregulation in WT mice. FIG. 12B: Detection of BFP2 reporter in AAV scramble and AAV shBSN mice, confirming the widespread expression of both sequences. FIG. 12C: H&E staining of WT mice injected with scramble and shBSN shRNA. FIG. 12D: BSN and Syn-1 immunofluoresce in WT mouse cortexes injected scramble and shBSN shRNA, and mean intensity of BSN and Syn-1. Data are shown as the mean±s.e.m. Experiments were performed with n=8 (FIG. 12A-D). Significance was determined by unpaired Student's t-test (FIG. 12A) and one-way ANOVA (FIG. 12D).

FIG. 13A-I shows reducing BSN levels mitigate tau pathology in male and female PS19 mice. FIG. 13A, B: Western blot (FIG. 13A) and quantification (FIG. 13B) of BSN downregulation in PS19 mice. FIG. 13C: Quantification of MC1 immunostaining in shBSN and scramble, of PS19 mice by sex as percentage of area. FIG. 13D, E: Quantification of hippocampal immunofluorescence of GFAP (FIG. 13D) and IBA1 (FIG. 13E) in WT_{scramble}, WT_{shBSN}, PS19_{scramble} and PS19_{shBSN} mice, separated by sex. FIG. 13F, G: Western blot quantification of pTauS396/5404 (PHF1) (FIG. 13F) and pTau Thr231 (FIG. 13G) in shBSN and scramble PS19 mouse brain lysate, separate by sex. FIG. 13H, I: Total human tau levels by ELISA (FIG. 13H) and tau-seeding activity (FIG. 13I) in shBSN and scrambled PS19 mouse brain lysates, separated by sex. Data are shown as the mean±s.e.m. Experiments were performed with n=8 (FIG. 13A, B) and n=4 (FIG. 13C-I). Significance was determined by unpaired Student's t-test (FIG. 13B, C, F-I) and one-way ANOVA (FIG. 13D, E).

FIG. 14A, B show the increased susceptibility to PK degradation of misfolded tau aggregates after BSN downregulation in PS19 mice. FIG. 14A, B: Proteinase K sensitivity assay (FIG. 14A) and quantification (FIG. 14B) of MC1 IHC intensity at 0, 10 and 45 sec after PK treatment in scramble and shBSN PS19 brain sections. Scale bar: 100 μm for main pictures and 10μ for insets. Data are shown as the mean±s.e.m. Experiments were performed with n=3 (FIG. 14A, B). Significance was determined by unpaired Student's t-test (FIG. 14B).

FIG. 15A-C show the rescue of synaptic integrity in PS19 after BSN downregulation. FIG. 15A: Pearson's correlation of PSD95 and Syn-1 in WT_{scramble}, WT_{shBSN}, PS19_{scramble} and PS19_{shBSN} mice, separated by sex. FIG. 15B: Representative immunofluorescence in scramble and shBSN PS19 mice for misfolded tau (MC1), and Syn-1 protein. Colocalization analysis was performed to determine pixel intensity correlation between MC1 and Syn-1. Scale bar: 50 μm for main panels and 10 μm for insets. FIG. 15C: Pearson's correlation of MC1 and Syn-1 immunostainings in scramble and shBSN PS19 mice. Data are shown as the mean±s.e.m. Experiments were performed with n=4 (FIG. 15A) and n=8 (FIG. 15B, C). Significance was determined by one-way ANOVA (FIG. 15A) and unpaired Student's t-test (FIG. 15C).

FIG. 16A-J show that BSN downregulation improves behavioral and electrophysiological impairments in PS19 male and females. FIG. 16A, B: Average of fEPSP slope from minute 60 to 70 by sex (FIG. 16A) and paired-pulse ratio (PPR) (FIG. 16B) in 6-month-old WT and PS19 mice injected with shBSN or scramble shRNA. FIG. 16C-F: 2-paw test (FIG. 16C), 4-paw test (FIG. 16D), body temperature (FIG. 16E) and frailty test (FIG. 16F) in 9-month-old WT and PS19 mice injected with shBSN or scramble shRNA. FIG. 16G-J: Quantification of MC1 immunostaining as percentage of area (FIG. 16G), pTauS396/5404 (PHF1) protein levels (FIG. 16H), pTau Thr231 protein levels (FIG. 16I), and total human tau levels by ELISA (FIG. 16J) in shBSN and scrambled PS19 male and female mouse brain lysates. Data represent the mean±s.e.m. Experiments were performed with n=5 for males and n=6 for females for WT_{scramble}, n=5 for males and n=5 for females for PS19_{scramble}, n=5 for males and n=5 for females for WT_{shBSN}, and n=5 for males and n=6 for females for PS19_{shBSN} mice (FIG. 16A-B), n=10 for males and n=10 for females for WT_{scramble}, n=9 for males and n=12 for females for PS19_{scramble}, n=13 for males and n=9 for females for WT_{shBSN}, and n=8 for males and n=8 for females for PS19_{shBSN} mice (FIG. 16C-F), and n=4 per sex (FIG. 16G-J). Significance was determined by one-way ANOVA (FIG. 16A-F) and unpaired Student's t-test (FIG. 16G-J).

FIG. 17A-C depicts the downregulation of BSN corresponds to reduced ventricular volume and an increment of hippocampal volume in PS19 mice. FIG. 17A-C: In vivo Magnetic resonance imaging (MRI) (FIG. 17A), quantification of ventricular (FIG. 17B), and hippocampal volume normalized to total brain volume (FIG. 17C) in 9-month-old WT and PS19 mice injected with shBSN or scramble shRNA. Data represent the mean±s.e.m. Experiments were performed with n=7 for WT_{scramble}, n=8 for PS19_{scramble}, n=4 for WT_{shBSN}, and n=7 for PS19_{shBSN} mice. Significance was determined by one-way ANOVA.

FIG. 18A,B depicts that siRNA against BSN downregulated BSN levels in neuronal primary cultures. FIG. 18A) Representative Western blot of cell lysate from neurons treated with siRNA anti-Bassoon. Vinculin was used as loading control. FIG. 18B) Quantification of Bassoon protein levels measure by Western blot. n=4, *p<0.05 and **p<0.01.

FIG. 19A,B shows the results of siRNA against BSN downregulated BSN levels in neuronal primary cultures. FIG. 19A) Double staining of neurons treated with siRNA against Bassoon using an anti-BSN antibody (red) and anti-MAP2 antibody (green). FIG. 19B) Quantification of BSN protein levels measure by percentage (%) of BBSN staining per cellular area determine by MAP2 staining. n=5.

### DETAILED DESCRIPTION

Various quantities, such as amounts, sizes, dimensions, proportions, and the like, are presented in a range format throughout this disclosure. It should be understood that the description of a quantity in range format is merely for convenience and brevity and should not be construed as an inflexible limitation on the scope of any embodiment. Accordingly, the description of a range should be considered to have specifically disclosed all the possible subranges as well as all individual numerical values within that range unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 4.62, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is to describe particular embodiments only and is not intended to be limiting of any embodiment. As used herein, the singular forms “a,” “an”, and “the” are intended to include the plural forms as well, unless the context clearly indicates otherwise. It will be further understood that the terms “includes”, “comprises”, “including” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Unless expressly stated or obvious from context, as used herein, the term “about” in reference to a number or range of numbers is understood to mean the stated number and numbers +/−10% thereof, or 10% below the lower listed limit and 10% above the higher listed limit for the values listed for a range.

In any of the embodiments disclosed herein, the terms “treating” or “to treat” includes restraining, slowing, stopping, or reversing the progression or severity of an existing symptom or disorder.

In any of the embodiments disclosed herein, the term “patient” refers to a human.

### Excipients

Illustrative, non-limiting examples of excipients or carriers include sodium citrate or dicalcium phosphate and/or a) one or more fillers or extenders (a filler or extender may be, but is not limited to, one or more selected from starches, lactose, sucrose, glucose, mannitol, and silicic acid), b) one or more binders (binders may be selected from, but not limited to, carboxymethylcellulose, alginates, gelatin, polyvinylpyrrolidinone, sucrose, and acacia), c) one or more humectants (a humectant may be, but is not limited to, glycerol), d) one or more disintegrating agents (disintegrating agents may be selected from, but are not limited to, agar-agar, calcium carbonate, potato or tapioca starch, alginic acid, silicates, and sodium carbonate), e) one or more solution retarding agents (for example, but not limited to, paraffin), f) one or more absorption accelerators (selected from, but not limited to, quaternary ammonium compounds), g) one or more wetting agents (for example, but not limited to, acetyl alcohol and glycerol monostearate), h) one or more absorbents (selected from, but not limited to, kaolin and bentonite clay), and i) one or more lubricants (selected from, but not limited to, talc, calcium stearate, magnesium stearate, solid polyethylene glycols, and sodium lauryl sulfate). In the case of capsules, tablets and pills, for example, the dosage form may also comprise buffering agents.

### “Effective or Therapeutic Amount”

Effective or therapeutic amounts of the compositions of this disclosure include any amount sufficient to inhibit (e.g., slow or stop) the progression of a neurodegenerative disorder. In some embodiments, effective amounts of the compositions include any amount sufficient to inhibit (e.g., slow or stop) the deterioration of the muscular function of a patient.

The amount of the active ingredient that may be combined with the optional carrier materials to produce a single dosage form may vary depending upon the host treated and the particular mode of administration. The specific dose level for any particular patient may depend upon a variety of factors, including the activity of the specific compound employed, the age, body weight, general health, sex, diet, time of administration, route of administration, rate of excretion, drug combination, and the severity of the particular disorder or disease undergoing therapy. A therapeutically effective amount for a given situation can be readily determined by routine experimentation and is within the skill and judgment of the ordinary clinician.

The term “RNA interference” or “RNAi” refers to the silencing or decreasing of gene expression by siRNAs. It is the process of sequence-specific, post-transcriptional gene silencing in animals and plants, initiated by siRNAthat is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

Pathological tau aggregation is a defining histopathological feature of Alzheimer's disease (AD) and other neurodegenerative diseases collectively known as tauopathies. The propagation of pathological forms of tau in AD patient brains has been shown to follow neuronal networks. However, the cellular mechanisms involved in tau propagation and the nature of the tau species involved in spreading remain unclear. The inventors have identified the proteins that specifically interact with this tau seed.

The current invention discloses the identification of bassoon (BSN), a scaffolding presynaptic protein, as an interactor of the tau seed isolated from a mouse model of tauopathy, and from AD and progressive supranuclear palsy (PSP) postmortem samples. It is shown that BSN exacerbates tau seeding and toxicity in vivo and that BSN downregulation significantly decreases tau spreading and overall disease pathology in vivo, rescuing synaptic and behavioral impairment and ameliorating brain atrophy.

The invention provides methods using interactors, including BSN, to stabilize tau seeds as a therapeutic approach for neurodegenerative tauopathies including Alzheimer's disease and related disorders including chronic traumatic encephalopathy (CTE).

More specifically, the current invention provides methods of treating neurodegenerative tauopathies in a patient in need thereof comprising administration of an agent that reduces or inhibits the interaction of a tau seed interactor with intracellular tau proteins.

In some embodiments, the tau seed interactor is Bassoon (BSN), a scaffolding protein of the presynaptic active zone. In some embodiments, the agent comprises an adeno-associated virus (AAV)-containing an a short-hairpin RNA (shRNA) against BSN (shBSN).

In some embodiments, the shBSN sequence is CCTAACGCTTTCCTCTGACAT (SEQ ID NO: 1).

In other embodiments, the shBSN can be attached to or part of any vector known in the art including plasmids, viral vectors, bacteriophages, cosmids, and artificial chromosomes.

In other embodiments, the agent comprises a monoclonal antibody directed against the tau seed interactor. In yet other embodiments, the agent comprises a monoclonal antibody directed against BSN. In still other embodiments, the agent is an siRNA or antisense oligonucleotide that targets BSN.

In any of the embodiments, the agent that reduces or inhibits the interaction of a tau seed interactor with intracellular tau proteins leads to a reduction of neurodegeneration in a patient with a neurodegenerative tauopathies.

In any of the embodiments, the agent that reduces or inhibits the interaction of a tau seed interactor with intracellular tau proteins leads to a behavioral improvement in a patient with a neurodegenerative tauopathies.

The current invention also provides methods of preventing neurodegenerative tauopathies in a patient in need thereof comprising administration of an agent that reduces or inhibits the interaction of a tau seed interactor with intracellular tau proteins.

In any of the embodiments described herein, the neurodegenerative tauopathies can be neurodegenerative diseases characterized by abnormal metabolism of misfolded tau proteins which in turn leads to intracellular accumulation and formation of neurofibrillary tangles (NFT). In any embodiment, the neurodegenerative tauopathies are selected from Alzheimer's disease, progressive supranuclear palsy (PSP), frontotemporal lobar degeneration (FTLD-TAU), corticobasal degeneration, Pick's disease (frontal temporal dementia), chronic traumatic encephalopathy (CTE), and primary age related taupathy.

The current invention also provides methods of treating or preventing Alzheimer's disease in a patient in need thereof comprising administration of an agent that reduces that reduces the interaction of a tau seed interactor with intracellular tau proteins.

In yet other embodiments, current invention provides methods of treating or preventing chronic traumatic encephalopathy (CTE) in a patient in need thereof comprising administration of an agent that reduces the interaction of a tau seed interactor with intracellular tau proteins.

The current invention provides pharmaceutical compositions comprising an agent that reduces the interaction of a tau seed interactor with intracellular tau proteins. The pharmaceutical compositions of the current invention can further comprise one or more pharmaceutically acceptable carriers, diluents or excipients.

In any of the methods of the current invention, can further comprise the administration of one or more additional therapeutic agents.

### General Methods and Assays

### Mice

Mice were bred and housed at the Indiana University School of Medicine (IUSM) animal care facility and were maintained according to USDA standards (12-h light/dark cycle, food and water ad libitum) in accordance with the Guide for the Care and Use of Laboratory Animals (National Institutes of Health, Bethesda, MD). The PS19 mouse model, which overexpresses human 1N4R tau with the P301S mutation on C57B6/J background, was directly purchased from The Jackson Laboratory (stock number 008169). For all described experiments, 4, 6 and 9-month-old PS19 and wild-type (WT) littermates of both sexes were utilized. All mice were randomly assigned to experiments. Animals were anesthetized and euthanized according to IUSM Institutional Animal Care and Use Committee-approved procedures.

### Human Tissue Samples

Frozen block from post-mortem brain tissues from AD subjects, PSP subjects and control subjects were provided by the Brain Resource Center at Johns Hopkins. AD cases consisted of pathologically severe AD, stage V-VI.

### Preparation of TBS-Soluble Homogenates

Each brain tissue was homogenized in 1:10 (w/v) TBS buffer with a protease inhibitor cocktail (Roche, 11873580001). Samples were then centrifuged at max speed for 15 min at 4° C. The supernatants were portioned into aliquots, snap-frozen and stored at −80° C. until analyzed.

### Size Exclusion Chromatography

Size exclusion chromatography was performed with a Superose 6 Increase 10/300 GL column (GE Healthcare, 29091596) on an ÄKTA pure 25 L chromatography system (GE Healthcare, 29018224). The column was equilibrated with 1.5 CV of a 50 mM NaCl, 50 mM Tris pH 8.0 buffer at a flow rate of 0.7 mL/min. Samples were clarified by centrifugation at 10,000 g for 10 min. Protein concentration was quantified by Bradford assay, and 1-5 mg total protein of supernatant was taken for separation, depending on the sample. The supernatant was concentrated with a 0.5 mL 3K Amicon centrifugal filter (Millipore Sigma, UFC5003) to ˜200 μL, then loaded onto the column via sample loop injection. Starting from injection, 1 mL fractions were collected into tubes containing EDTA-free protease inhibitor (Roche, 11873580001) at a flow rate of 0.3 m/min.

### Tau Seeding Assay

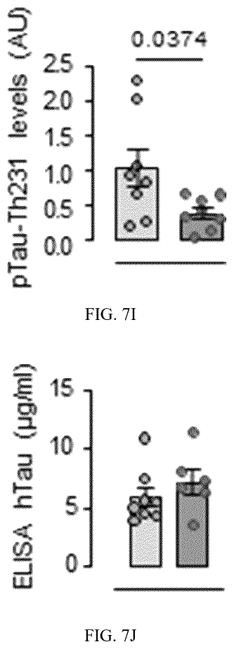

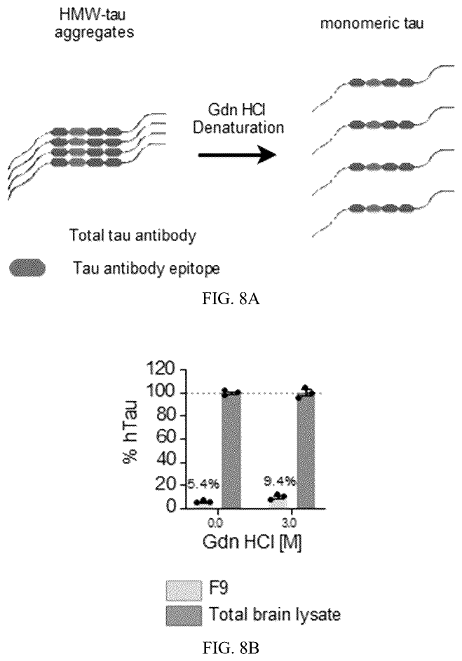

Seeding assay was performed as previously described (Holmes et al., 2014) with minor modifications. TauRD P301S FRET Biosensor cells (ATCC #CRL-3275) were plated at 35,000 cells/well in 130 μL media in a 96-well plate, then incubated at 37° C. overnight. The next day, cells were transfected with cell or brain lysate (20 μg total protein per well) by using Lipofectamine 2000 then incubated at 37° C. for 48 hr. Cells were harvested by trypsinization. Flow cytometry was conducted with a BD LSRFortessa™ X-20 with a High Throughput Sampler. The BV421 channel (Ex: 405 nm, Em: 450/50) was used to detect CFP, and the BV510 channel (Ex: 405 nm, Em: 525/50+505LP) was used to detect FRET signal, with compensation to remove the CFP spillover into the FRET channel. Data analysis was performed with FlowJo, using gating strategy shown in FIG. 8. Seeding was quantified by integrated FRET density, defined as the product of the percentage of FRET-positive cells and median fluorescent intensity (MFI) of FRET-positive cells.

### Human Tau ELISA

ELISA was performed on SEC fractions using Tau (Total) Human ELISA Kit (Invitrogen, KHB0041) by following the directions provided by the manufacturer. Lysates were diluted 1:50,000 in blocking buffer. F7-F14 were diluted 1:2,000 in blocking buffer. F15-F22 were diluted 1:20,000 in blocking buffer.

### Guanidine HCl Denaturation Assay

Each sample was diluted to the same protein concentration by Bradford assay. Samples were denatured by adding a Gdn HCl solution at a 1:1 ratio by volume, varying the initial concentration of the Gdn HCl solution to obtain the desired final concentration after mixing. The mixture was incubated for 30 min at room temperature (RT). After incubation, the mixture was diluted for ELISA or Western blot immediately. To control for the potential effect of trace amount (<5 mM) of Gdn HCl on the ELISA assay, a 1 ng/mL tau standard was also spiked with Gdn HCl at a concentration matching that of each sample. The effect of trace Gdn HCl on the assayed ELISA concentration was not statistically significant.

### Immunoprecipitation

From HEK293T lysates, SEC fraction 9 from PS19 mice, AD and PSP cases, tau was immunoprecipitated by using 2 μg of biotinylated HT7 antibody for every 100 ng of human tau quantified by ELISA. IgG isotype control antibodies were used for comparison. The IP and flow-through samples were then subjected to further analysis.

### Electron Microscopy

IP samples were analyzed by negative stain biological TEM as described before. See Sanyal et al., “Alpha-Synuclein Is a Target of Fic-Mediated Adenylylation/AMPylation: Possible Implications for Parkinson's Disease,” (2019) J Mol Biol 431: pp. 2266-2282. Briefly, 3 μl of the IP sample was directly pipetted on a discharged carbon-coated copper TEM grids and incubated for 1 min. Grids were carefully washed with deionized water without letting it dry and stained with 3.5 μL of 1% (w/v) phosphotungstic acid (PTA) solution for 1 min. Any excess solution was then removed by blotting with a Whatman filter paper. The samples were imaged using an FEI Tecnai T12 Transmission Electron Microscope operating at 80 kV. Images were captured using Gatan digital micrograph software and the width was measured using image analysis software ImageJ. Three independent samples were analyzed, and results were plotted with Prism 9.0 software.

### Mass Spectrometry

Sample preparation for the affinity purification mass spectrometry experiments were designed at the Proteomics Core Facility of Indiana University School of Medicine (IUSM) from previously reported qPLEX-RIME methodology that have integrated isobaric labelling and tribrid mass spectrometry methods with RIME (Rapid Immunoprecipitation Mass spectrometry of Endogenous proteins) with modifications. See Justice et al., “Mutant thermal proteome profiling for characterization of missense protein variants and their associated phenotypes within the proteome,” (2020) J Biol Chem 295: pp. 16219-16238; Mohammed et al., “Rapid immunoprecipitation mass spectrometry of endogenous proteins (RIME) for analysis of chromatin complexes,” (2016) Nat Protoc 11: pp. 316-326; Papachristou et al., “A quantitative mass spectrometry-based approach to monitor the dynamics of endogenous chromatin-associated protein complexes,” (2018) Nat Commun 9: p. 2311. Briefly, magnetic beads, affinity captured with proteins, were treated with 10 μL trypsin/LysC (15 ng/μL, Promega Corporation, WI, U.S.A.) in 100 mM ammonium bicarbonate (ABC) and incubated overnight at 37° C. with shaking, followed by a second digestion with trypsin/LysC at RT for 4 hr. The magnetic beads were next separated on a magnetic stand to separate the supernatant peptide solution. De-salting was carried out using Sep-Pak® Vac 1cc C18 Cartridges, (Waters Corporation Milford, MA, U.S.A.) employing a vacuum manifold. All samples were then dried using a SpeedVac, reconstituted in 50 mM triethylammonium bicarbonate (TEAB) and subjected to TMT (Tandem Mass Tags) based labelling using TMT10plex reagents (ThermoFisher, cat. No. 90309). Next, Nano-LC-MS/MS analyses were performed on an Orbitrap Fusion™ Lumos™ mass spectrometer (Thermo Fisher Scientific) coupled to an EASY-nLC™ HPLC system (Thermo Scientific). Labeled, mixed and dried peptide samples were reconstituted in 0.1% formic acid (20 μL) and 8 μL equivalent volume was loaded onto a reversed phase PepMap™ RSLC C18 column (2 μm, 100 Å, 75 μm×50 cm) with Easy-Spray tip at 750 bar applied maximum pressure. The peptides were eluted using a varying mobile phase (MP) gradient from 94% phase A (FA/H₂O 0.1/99.9, v/v) to 28% phase B (FA/ACN 0.1/99.9, v/v) for 160 min; to 35% phase B for 5 min; to 50% phase B for 14 min to ensure elution of all peptides and bringing down the MP-composition to 10% phase B for 1 min at 400 nL/min to bring the MP-composition to higher % of phase A. Nano-LC mobile phase was introduced into the mass spectrometer using an EASY-Spray™ Source (Thermo Scientific™) During peptide elution, the heated capillary temperature was kept at 275° C. and ion spray voltage was kept at 2.6 kV. The mass spectrometer method was operated in positive ion mode for 180 min having a cycle time of 4 sec. MS data was acquired using a data-dependent acquisition method that was programmed to have 2 data dependent scan events following the first survey MS scan. During MSn level 1, using a wide quadrupole isolation, survey scans were obtained with an Orbitrap in the range of 375-1500 m z at 60k resolution. To isolate and fragment the selected precursor ions, MSn level 2 scans were performed with following vendor defined parameters: Isolation mode=Quadrupole; Isolation Offset=Off; Isolation Window=0.8; Multi-notch Isolation=False; Scan Range Mode=Auto Normal; First Mass=100; Activation Type=HCD; Collision Energy Mode=Fixed; Collision Energy (%)=38; Detector Type=Orbitrap; Orbitrap Resolution=50k; Maximum Injection Time=90 ms; AGC Target=1E5; Data type=Centroid; Polarity=Positive; Source Fragmentation=False. The data were recorded using Thermo Scientific Xcalibur (4.1.31.9) software (©2017 Thermo Fisher Scientific Inc.). Resulting RAW files were analyzed using Proteome Discover 2.2.0.388 (Thermo Fisher Scientific). The MS/MS spectra were searched against in silico tryptic digest of a database (FASTA) downloaded from UniProt (mouse_human_uniprot_contam_030419.fasta) using the SEQUEST HT search engine. The following search parameters were applied: Trypsin as the proteolytic enzyme; peptides with a maximum number of 2 missed cleavages, precursor mass tolerance of 10 ppm, and a fragment mass tolerance of 0.6 Da. Static modifications used for the search were, 1) carbamidomethylation on cysteine(C) residues; 2) TMT sixplex label on lysine (K) residues and the N-termini of peptides. Dynamic modifications used for the search were oxidation of methionines and phosphorylation of S/T/Y. Percolator False Discovery Rate was set to a strict setting of 0.01 and a relaxed setting of 0.05. Values from both unique and razor peptides were used for quantification. Finally, the mass spectrometry proteomics data have been deposited to the ProteomeXchange Consortium via the PRIDE5-7 partner repository with the dataset identifier PXD027451 and 10.6019/PXD027451. See Perez-Riverol et al., “The PRIDE database and related tools and resources in 2019: improving support for quantification data,” (2019) Nucleic Acids Res 47: pp. D442-D450; Deutsch et al., “The ProteomeXchange consortium in 2020: enabling ‘big data’ approaches in proteomics,” (2020) Nucleic Acids Res 48: pp. D1145-D1152; Perez-Riverol et al., “PRIDE Inspector Toolsuite: Moving Toward a Universal Visualization Tool for Proteomics Data Standard Formats and Quality Assessment of ProteomeXchange Datasets,” (2016) Mol Cell Proteomics 15: pp. 305-317.

### Enrichment Analysis

Abundance ratios of HT7 IP were compared with control IgG IP to determine protein enrichment levels (n=3). Proteins enriched by a fold change >4 with p<0.05 by t-test (total of 1159 in tau seed and 365 in tau monomer) were selected for DAVID functional annotation (https://david.ncifcrf.gov/) using default settings with the mouse genome. We then compared both interactomes with the “Wall of Targets” nominated by the AMP-AD as possible targets for AD (https://agora.ampadportal.org/genes/(genes-router:genes-list)). Synaptic proteins were identified by searching Gene Ontology for genes that have a “part of” or “regulates” transitive closure relation with the synapse term (GO:0045202).

### Immunohistochemistry and Proteinase K Sensitivity Assay

Slides were deparaffinized and rehydrated in several incubations of xylene, ethanol gradient (100% to 30%) and deionized water. The slides were stained with hematoxylin and eosin (Vector Labs, H-3502) following the manufacture's protocol. Slides were dehydrated and coverslip mounted for imaging. For Proteinase K sensitivity assay, following antigen retrieval step, the slides were incubated with 50 μg/mL PK (Bioline, BIO-37037), 10 mM Tris HCl pH 7.8, 100 mM NaCl, 0.1% NP-40 at 37° C. for 0 sec, 10 sec and 45 sec, similar to what was described before8. Bengoa-Vergniory, N. et al. “Tau-proximity ligation assay reveals extensive previously undetected pathology prior to neurofibrillary tangles in preclinical Alzheimer's disease,” (2021) Acta Neuropathologica Commun 9: p. 18.

### Disorder & Hydrophobicity Profile

Disordered residues were predicted by ESpritz9 using the DisProt database. See Walsh et al., “ESpritz: accurate and fast prediction of protein disorder,” (2011) Bioinformatics 28: pp. 503-509. The optimal binary decision threshold Sw was used as a threshold for the disorder. The hydrophobicity profile was calculated using the method of Kyte & Doolittle with a moving average window of 9 residues. See Kyte et al., “A simple method for displaying the hydropathic character of a protein,” (1982) J Mol Biol 157: pp. 105-132.

### Immunofluorescence

Mouse paraffin sections were deparaffinized in xylene, rehydrated in ethanol gradient (100% to 30%), and washed with deionized water. Then, the sections were heated to 95° C. in high pH antigen retrieval solution (Invitrogen eBioscience, 00-4956-58) for 10 min with a microwave oven. After washing twice with PBS (5 min each), the sections were incubated with TrueBlack (Biotium, 23007) for 3 min and then washed 3 times in PBS. Then, tissues were blocked with PBS 10% goat serum and 0.01% Triton X-100 for 1 hr at RT. Sections were then incubated overnight at 4° C. with the primary antibodies. The next day, sections were quickly washed 3 times in PBS and incubated 2.5 hr with 1:500 Alexa Fluor antibodies, diluted in blocking solution. Sections were quickly washed 3 more times in PBS and mounted with Fluoromount (Sigma, F4680). For AD and PSP cases, frozen sections were first fixed with paraformaldehyde (PFA) 4% 1 hr at RT and then permeabilized in Triton X-100 0.01% for 1 hr, continuing with the mentioned immunofluorescence protocol. For cell immunofluorescences, cells were fixed in 4% paraformaldehyde for 30 min and washed 3 times for 5 min with 1×PBS. Cells were permeabilized with 0.010% Triton-X for 20 min at RT and washed 3 times with 1×PBS. Coverslips were incubated with 10% Normal Goat Serum and blocked for 30 min at RT. Then, the coverslips were incubated with primary antibodies diluted in 10% Normal Goat Serum overnight at 4° C. The next day, coverslips were washed 3 times with 1×PBS and incubated with Alexa secondary antibodies diluted in 10% Normal Goat Serum for 2 hr at RT. Coverslips were then washed 3 times with 1×PBS and mounted using Fluoromount.

### Proximity Ligation Assay (PLA)

HEK cells were grown on coverslips and transfected with plasmids coding for TauWT, TauP301S, full-length BSNWT and BSN N- and C-terminal fragments. 72 hr later, cells were fixed in 4% paraformaldehyde for 30 min and washed 3 times for 5 min with PBS. PLA was performed using the Duolink In Situ Fluorescence kit (Sigma, DU092101) as per the manufacturer's instructions. Briefly, the coverslips were incubated with the Duolink Blocking solution for 1 hr at 37° C. in a humidified chamber. Then, the blocking solution was removed, and coverslips were incubated with primary antibodies diluted in Duolink Antibody Diluent overnight at 4° C. The coverslips were then washed with Wash Buffer A (Sigma) twice for 5 min. Then, the Duolink In Situ PLA Probe Anti-Rabbit PLUS and Duolink In Situ PLA Probe Anti-Mouse MINUS PLA probes (Sigma) diluted in Duolink Antibody Diluent were applied to the coverslips and incubated in a humidified chamber for 1 hour at 37° C. Coverslips were washed twice with Wash Buffer A, followed by incubation with the ligation solution for 30 min at 37° C., then washed twice with Wash Buffer A and incubated with amplification solution for 100 min at 37° C. Finally, the coverslips were washed twice for 10 min in Wash Buffer B (Sigma), once in 0.01% Wash Buffer B and then mounted onto the slides using the Duolink In Situ Mounting Medium with DAPI (Sigma). The edges were sealed with clear nail polish.

### Imaging and Analysis

Mice sections were imaged with a Nikon A1-R laser scanning confocal microscope coupled with Nikon AR software. For tau propagation analysis, images (z-stack 0.5 μm stepsize) were imported into ImageJ (NIH). Propagation analysis was performed by counting HT7(+)/GFP(−) or GFP(+) cells per 1 mm². For functional synapsis analysis, image stacks (0.1 μm) were imported into Image J software, and colocalization was quantified by Pearson's correlation coefficient using JACoP plugin.

### Cell Culture Co-Transfection

HEK293T cell line was cultured in DMEM (Invitrogen) with 10% FBS (Invitrogen). Human Tau-P301L/WT and wild-type human Bassoon were cloned in pRK5 and CMV-plasmid (Vector Builder) respectively. Plasmids were transfected, in a ratio TauP301L: BSN—1:10, with Lipofectamine 3000 (Invitrogen) and incubated for 72 hr. Cells were lysed in 1×TBS with protease inhibitor (Roche) by sonication (2 min, 30% Amp, 5 sec ON −5 sec OFF). The lysate was centrifuged at 21,100 g for 10 min at 4° C. The supernatant (TBS soluble fraction) was transferred into fresh tubes and used for downstream seeding activity, WB and IP analysis.

### Western Blot Analysis

TBS soluble samples were incubated with Laemmli SDS buffer, reducing 6× (Alfa Aesar J61337) at 95° C. for 10 min for denaturing conditions or 4×NuPAGE sample buffer (Invitrogen NP0007) for non-denaturing and then loaded on 4-12% NuPAGE Novex gels (Invitrogen). Nitrocellulose membranes were used to transfer proteins and blocked with 5% BSA in TBS with 0.01% tween followed by overnight incubation of primary antibodies diluted in the blocking solution. HRP secondary antibodies were incubated for 1 hr at RT and the proteins were detected with Supersignal West Pico (Thermo Scientific) and imaged by using iBright 1500 (Invitrogen).

### Drosophila Stocks and Genetics

Drosophila melanogaster stocks and crosses were maintained on Nutri-fly Bloomington formulation (Genesee Scientific) at 25° C. in a 12-hr light/dark cycle. All the fly experiments were performed at day 30 post-eclosion. Transgene overexpression was achieved with the Gal4/UAS system, using GMR-Gal4 (BDSC, #9146) and GMR-TauP301L (BDSC, #51377) lines. See Karsten et al., “A Genomic Screen for Modifiers of Tauopathy Identifies Puromycin-Sensitive Aminopeptidase as an Inhibitor of Tau-Induced Neurodegeneration,” (2006) Neuron 51: pp. 549-560. For human BSN overexpression, full-length BSN wild-type and P3866A mutant sequences were cloned downstream of the Gal4-responsive upstream activating sequences into the pUAST plasmid (Vectorbuilder) and then microinjected in fly embryos (Bestgene).

### Eye Phenotype Quantification, WB and Seeding

For light microscope imaging, adult flies were immobilized at −80° C., and then mounted for visualization. Flies were then imaged using a Leica DMC6200 camera with a 10× objective, with a white light falling on each ommatidium until observing a single reflection spot in its center. Images were captured and stacked using Zerene Stacker (Zerene Systems, Richland, WA). For eye phenotypic scoring, Flynotyper plugin was used on Image J software as previously described. See Iyer et al. “Quantitative assessment of eye phenotypes for functional genetic studies using Drosophila melanogaster,” (2016) Biorxiv 036368: doi: 10.1101/036368.

### AAV Production and Injections

For shRNA, sequences for mouse Bsn shRNA (CCTAACGCTTTCCTCTGACAT) (SEQ ID NO: 1) and scramble shRNA (CCTAAGGTTAAGTCGCCCTCG) (SEQ ID NO: 2) were used. shRNA sequences were cloned downstream the U6 promoter and packaged into AAV9 from Vectorbuilder. For in vivo tau propagation experiments, the EGFP-P2A-hTau(2N4R)^{P301L} sequence was cloned downstream of the CMV promoter and packaged into AAV9 (Vectorbuilder).

For Bsn downregulation, neonatal (P0) PS19 and wild-type mice were injected with the AAV scramble or shRNA Bsn sequence. Animals were sacrificed at 4, 6 or 9 months after injections. For in vivo tau propagation experiments, neonatal mice were injected with AAV encoding either scramble or shRNA Bsn sequences. After twelve weeks, mice were intracranially injected with an AAV encoding EGFP-P2A-hTau(2N4R)^{P301L} into the hippocampus (1 μL in the left hemisphere). Mice were anaesthetized with isoflurane (2%) and AAV injections were made in bregma A/P—2 mm, M/L—1.5 mm, D/V—1.5 mm at 0.5 μl min−1. Head skin was sutured, and mice were allowed to recover on a warming incubator. Mice received meloxicam for two days after surgery.

### Electrophysiology

Hippocampal slices (300 μm-thick) were cut at 0.1 mm/s with a Leica VT1200 vibratome in ice-cold oxygenated external solution containing a sucrose-based artificial cerebrospinal fluid (aCSF) (in mM: Sucrose, 194; NaCl, 30; NaHCO₃, 26; Glucose, 10; KCl, 4.5; NaH₂PO₄, 0.5; MgCl₂, 1; pH 7.4) bubbled with 95% O₂/5% CO₂. Prior to cutting, mice were anesthetized with isoflurane and perfused transcardially with 10 mL of cold aCSF solution. The brains were quickly removed and blocked. After cutting, slices were transferred to an incubation chamber containing oxygenated aCSF solution at 33° C. for 1 hr. Slices were then kept at RT until recording. Recordings were done at 30-32° C. in a submersion chamber perfused (1-2 mL/min) with aCSF solution comprised of (in mM: NaCl, 124; KCl 4.5; NaH₂PO₄, 1.2; MgCl₂, 1; CaCl₂, 2; NaHCO₃, 26; and Glucose 10, continuously bubbled with 95% O2/5% CO₂; pH 7.4, 310 mOsm).

Input-Output (I/O), Paired-pulse ratio (PPR) and Long-term potentiation (LTP) recordings: For I/O curves and LTP experiments, field excitatory postsynaptic potentials (fEPSP) evoked by Schaffer collateral stimulation with stainless steel stereotrodes (1MΩ, P1 Technologies), were recorded in current-clamp mode with micropipettes filled with 1 M NaCl using a Multiclamp 700B amplifier and Clampex software (Molecular Devices). Signals were low pass filtered at 2 KHz and digitized at 50 KHz. The recording micropipette was placed in the CA1 region of the hippocampus. The intensity of the stimulator was increased stepwise until a maximal response was obtained using a constant current isolated stimulator (Digitimer). The slope of the fEPSP (mV/ms) was measured. PPR were obtained every 20 sec at 40 ms inter stimuli interval (ISI). The LTP protocol consisted of 10 min of stable baseline: 30 pulses every 20s (stimulus strengths were adjusted to evoked <50% of the maximal response); 1 min conditioning: trains (10 pulses at 100 Hz) repeated 4 times every 20 s; then a 60 min post-conditioning at the same baseline stimulation frequency: The synaptic strength change was measured from the slope of the fEPSP and data were expressed as a percentage of change with respect to the average baseline.

### Behavioral Testing

Body temperature, body weight and frailty: Mice were weighed, and the resting core body temperature was measured by inserting a lubricated rectal probe (Bioseb), ˜1 cm into the rectum for 10 sec. For the frailty examination, mice were assessed for the presence or absence of 26 difference characteristics as described previously. See Whitehead et al., “A Clinical Frailty Index in Aging Mice: Comparisons with Frailty Index Data in Humans,” (2014) Journals Gerontology Ser 69: pp. 621-632. A score of 0 was given if the mouse had no sign of the deficit, 0.5 if there was a mild deficit and 1 if there was a severe deficit.

Grip Strength: Grip strength of the forelimbs (front two paws) and all limbs (four paws) was evaluated using the Grip Strength Meter (Bioseb, BIO-GS3). As per the manufacturer's protocol, mice were held by the tail and lowered towards the apparatus and allowed to grab the metal grid using two or four paws. The mice were pulled backward horizontally, and the force applied to the grid just before they lost their grip was recorded as the peak tension (converted to grams by the transducer). Peak force was measured two times in succession for each mouse for the front two paws and all four paws. The mean value of both trials was used for analysis. Mice were given a minimum break of 5 min between trials.

### Magnetic Resonance Imaging

T2-weighted high resolution structural images were acquired in a horizontal bore 9.4 Tesla Biospec pre-clinical MRI system (Bruker BioSpin MRI GmbH, Germany) equipped with shielded gradients (maximum gradient strength=660 mT/m, rise time=4570 T/m/s) and 1H mouse cryogenic surface coil (Cryoprobe, Bruker, Biospin). 2D T2-Weighted (Bruker, T2 Turbo RARA) images were acquired using the following parameters: TE/TR=43.67/7600 ms, Rare factor=8, matrix size=256×256, voxel size=60×60×200 μm³, number of slices=72, slice thickness=200 μm, number of averages=6 and acquisition time=25 min. Mice were anesthetized under 3% isoflurane and positioned in an MRI compatible head holder to minimize motion artefacts. Anesthesia was then maintained at 1.5% isoflurane in 100% O₂ throughout imaging. Respiration rate was monitored using a pressure pad placed under the animal abdomen, and animal body temperature was maintained by a warming pad (37° C.) placed under the animal. The high resolution in vivo T2-W images were oriented to Badhwar hippocampal atlas space, corrected for noise and skull stripped using STAPLE algorithm. See Badhwar et al., “Impaired structural correlates of memory in Alzheimer's disease mice,” (2013) Neuroimage Clin 3: pp. 290-300; Coupe, P. et al., “An Optimized Blockwise Nonlocal Means Denoising Filter for 3-D Magnetic Resonance Images, (2008) IEEE T Med Imaging 27: pp. 425-441; Cardoso et al., “STEPS: Similarity and Truth Estimation for Propagated Segmentations and its application to hippocampal segmentation and brain parcellation,” (2013) Med Image Anal 17: pp. 671-684 (2013). The skull stripped brain volumes were corrected for B1 field inhomogeneity using N4 bias field correction algorithm and then non-linearly registered to Badhwar hippocampal atlas using Symmetric diffeomorphic image registration with cross-correlation (SyN) algorithm implemented in ANTs. See Tustison et al., “N4ITK: Improved N3 Bias Correction,” (2010) IEEE T Med Imaging 29: pp. 1310-1320; Avants et al., “A reproducible evaluation of ANTs similarity metric performance in brain image registration,” (2011) Neuroimage 54: pp. 2033-2044; Avants et al., “Symmetric diffeomorphic image registration with cross-correlation: Evaluating automated labeling of elderly and neurodegenerative brain,” (2008) Med Image Anal 12: pp. 26-41.

Third ventricle, fourth ventricle and lateral ventricle were combined as a single region of interest (ROI) in atlas space and then transformed to individual in vivo T2-W image space using inverse transform matrix and deformation map, which were generated during the forward registration. Using registered ROI (ventricle) as prior label and sample specific T2-W image as a reference image, additional improvement in registration/segmentation was achieved using ANTs Atropos tool. See Avants et al., “An Open Source Multivariate Framework for n-Tissue Segmentation with Evaluation on Public Data,” (2011) Neuroinformatics 9: pp. 381-400. For each sample, final segmentation results were manually inspected for miss registration. Using brain mask, total intra cranial volume (TICV) was extracted using FSL “fslstats” tool and using segmented ventricle, total ventricle volume was also extracted using FSL “fslstats” tool.

To investigate group differences in ventricle volume, general linear model (GLM) was used. The independent between-group assessment was corrected for the effect of TICV. A post-hoc test was conducted to further understand the sensitivity of ventricle size in terms of group-wise comparisons. The analysis was performed in SPSS (IBM, SPSS, Version 27). To account for multiple comparisons across 4 groups, false discovery rate (FDR) correction using Benjamini-Hochberg criterion (α=0.05) was used (PFDR<0.05).

### Statistics

All statistical analysis and graph designs were performed using GraphPad Prism 9 software. The results in column graphs represented average+/−SEM. Student's t-test, One-Way ANOVA and Two-Way ANOVA tests were performed as necessary. For all tests, alpha value of 0.05 was used to determine statistical significance.

Further reference is made to the following experimental examples.

### EXAMPLES

The following examples are provided for the purpose of illustrating various embodiments of the invention and are not meant to limit the present disclosure in any fashion. The present examples, along with the methods described herein are presently representative of preferred embodiments, are provided only as examples, and are not intended as limitations on the scope of the invention. Changes therein and other uses which are encompassed within the spirit of the disclosure as defined by the scope of the claims will occur to those skilled in the art.

### Example 1

BSN Protein Interacts with a HMW Tau Seed

Tau seeds were charactered in a PS19 mouse model that overexpresses human tau harboring the P301S mutation. See Yoshiyama et al., “Synapse loss and microglial activation precede tangles in a P301S tauopathy mouse model,” (2007) Neuron 53: pp. 337-351. In this model, seeding activity is detected as early as 2 months of age, several weeks sooner than any other pathological marker. See Holmes et al., “Proteopathic tau seeding predicts tauopathy in vivo,” (2014) Proc Natl Acad Sci USA 111: pp. E4376-4385. To assess the molecular weight of the tau species involved in seeding activity, the inventors performed size exclusion chromatography (SEC) on TBS-soluble brain extracts from 3-month-old PS19 mice, representing an early stage of pathology. The tau-seeding activity of each SEC-fraction was assessed by transfection into Tau RD P301S FRET biosensor cells and quantification of the integrated FRET density by flow cytometry (FIG. TA). See Holmes et al. 2014.

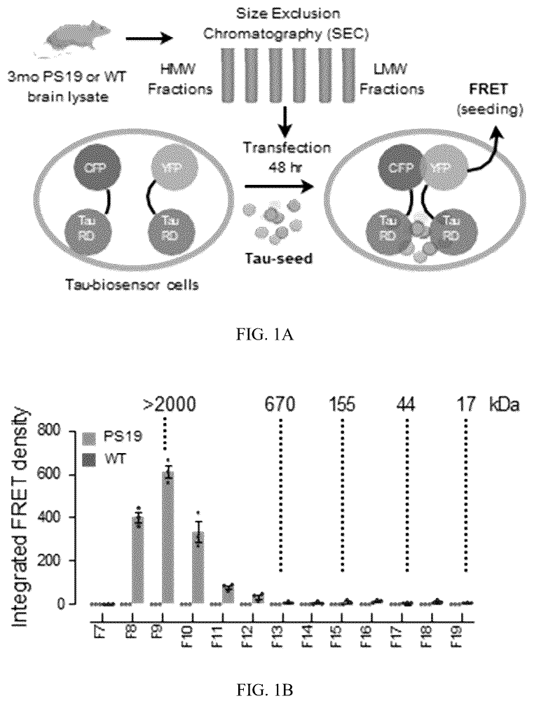

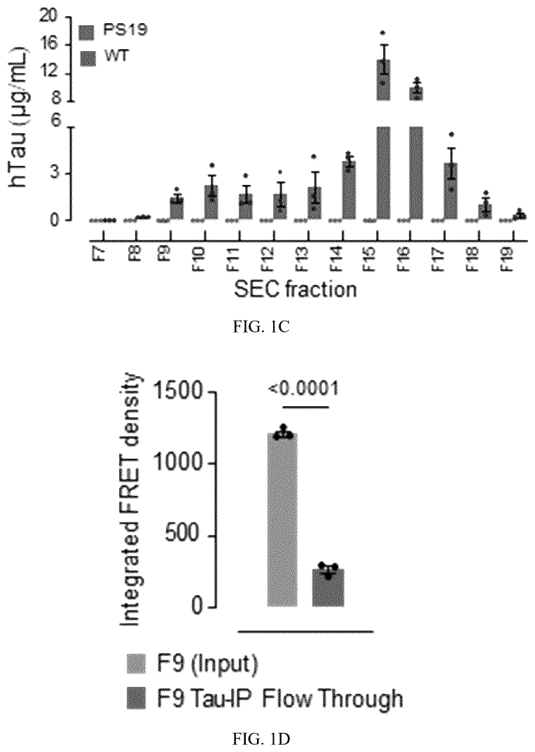

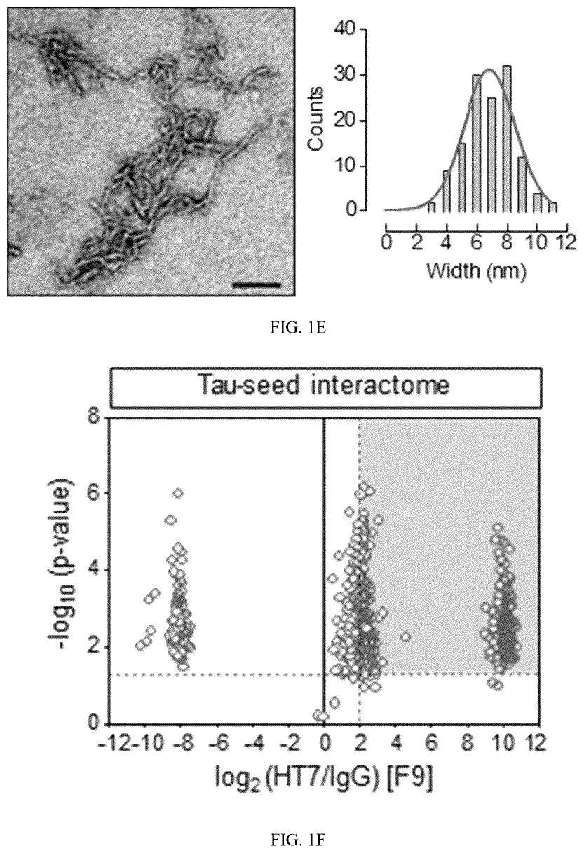

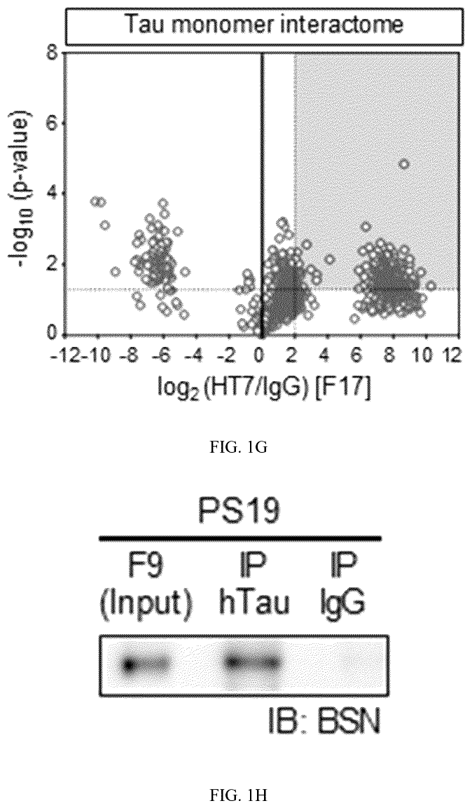

FIG. TA-H depicts the results of HMW tau seed interacting with BSN protein. FIG. TA is a schematic of the tau-seeding assay of SEC fractions. FIG. 1B shows tau-seeding activity of SEC fractions obtained from 3-month-old PS19 and wild-type (WT) mouse brain lysates. FIG. 1C: total human tau detected by ELISA in SEC fractions from PS19 and WT mouse brain lysates. FIG. 1D: tau seeding activity of the PS19 SEC fraction 9 (F9) containing HMW tau, before and after hTau immunoprecipitation (IP) using HT7 antibody. FIG. TE: Electron microscopy (EM) of the F9 IP product showing tau protofibril structures, and width distribution of protofibrils. Scale bar: 100 nm. FIG. 1F-G, Volcano plot indicating tau interactors found in F9 (FIG. 1F) and F17 (FIG. 1G) identified by LC-MS/MS. FIG. 1H: Western blot of BSN from co-immunoprecipitation (co-IP) of human tau (HT7) from F9 in PS19 brain lysates. Scale bar: 25 μm. Data are shown as the mean±s.e.m. Experiments were performed in triplicates (FIG. 1B-D, H), and significance was determined by unpaired Student's t-test (FIG. 1D).

FIG. 8A-B shows that HMW tau seed represents a low percentage of total tau in the PS19 mouse brain. FIG. 8A, Schematic of aggregated tau denaturation by guanidine HCl (Gdn HCl). FIG. 8B, hTau present in F9 (gray bars) as a percentage of total brain lysates (green bars) before and after denaturation with 3M of Gdn-HCl. Total human tau levels were assayed by ELISA. Data represent the mean±s.e.m. Experiment was performed with n=3.

The strongest seeding activity was found in the void volume fraction, fraction 9, which contained HMW proteins larger than 2,000 kDa (FIG. 1B). Fraction 9 contained only 5% of total tau, as assayed by ELISA for human tau (FIG. 1C, FIG. 8A,B). Interestingly, fraction 9 still contained a low percentage of total tau after treatment with the chaotropic denaturant guanidine HCl (Gdn-HCl) that unmasks hidden epitope meaning that the low total tau level detected in fraction 9 is not due to epitope masking in the HMW tau complex. See (FIG. 8A, B) and Hnasko et al., “Enhanced detection of infectious prions by direct ELISA from the brains of asymptomatic animals using DRM2-118 monoclonal antibody and Gdn-HCl,” (2018) J Immunol Methods 456: pp. 38-43; Kim et al., “Distinct populations of highly potent TAU seed conformers in rapidly progressing Alzheimer's disease,” (2022) Sci Transl Med 14: eabg0253. No seeding activity was observed in any SEC fractions from WT littermates (FIG. 1B).

To confirm that the tau species present in fraction 9 is responsible for the seeding activity, the inventors depleted human tau via immunoprecipitation (IP) using the HT7 antibody and observed a dramatic decrease in the seeding activity of the flow-through (FIG. 1D). Electron microscopy (EM) of the tau IP material from fraction 9 revealed that the seed takes on a predominantly curved linear structure with a width of 7±3 nm (FIG. 1E), defined as protofibrils. See Ono et al., “Alzheimer's disease as oligomeropathy,” (2018) Neurochem Int 119: pp. 57-70; Harper et al., “Models of amyloid seeding in Alzheimer's disease and scrapie: mechanistic truths and physiological consequences of the time-dependent solubility of amyloid proteins,” (1997) Annu Rev Biochem 66: pp. 385-407; Walsh et al., “Amyloid beta-protein fibrillogenesis—Detection of a protofibrillar intermediate,” (1997) J Biol Chem 272: pp. 22364-22372; Ramachandran et al., (2011) “Understanding the kinetic roles of the inducer heparin and of rod-like protofibrils during amyloid fibril formation by Tau protein,” J Biol Chem 286: pp. 38948-38959. Tau protofibrils are intermediate, metastable species that are more toxic and relevant to disease pathogenesis than larger, more stable fibrillar structures and neurofibrillary tangles. See Gerson et al., “Potential mechanisms and implications for the formation of tau oligomeric strains,” (2016) Crit Rev Biochem Mol Biol 51: pp. 482-496; Ghag et al., “Soluble tau aggregates, not large fibrils, are the toxic species that display seeding and cross-seeding behavior, (2018) Protein Sci 27: pp. 1901-1909.

Immunoprecipitation (IP) was also performed on human tau from fraction 9 and fraction 17 (containing monomeric tau) using the HT7 antibody to identify protein interactors of the HMW tau seed and monomeric tau isolated from the same brain. For this purpose, the IP products were analyzed by a TMT-tags based quantitative mass spectrometry workflow (FIG. 1F-G; FIG. 8A,B). DAVID functional annotation clustering revealed enrichment of synaptic terms in both interactomes and interestingly, many synaptic proteins identified as tau-seed interactors differed from those that interacted with monomeric tau. The relevance of synaptic interactors in tau pathogenesis is supported by an overwhelming volume of literature suggesting that the tau seed is propagated trans-synaptically. Moreover, a recent study demonstrated that wild-type tau interacted with synaptic proteins and that this interaction was enhanced by neuronal activity in iPSC-derived neurons. Both interactomes were compared with potential therapeutic targets for AD nominated by the AMP-AD in the “Wall of Targets.” See Hodes et al., “Accelerating Medicines Partnership: Alzheimer's Disease (AMP-AD) Knowledge Portal Aids Alzheimer's Drug Discovery through Open Data Sharing,” (2016) Expert Opin Ther Targets 20: pp. 389-391; Ertekin-Taner et al., “Identifying therapeutic targets for Alzheimer's disease with big data,” (2017) Neurodegener Dis Manag 7: pp. 101-105. From this comparison, the inventors identified bassoon (BSN), a large scaffolding protein of the presynaptic active zone involved in regulating synaptic neurotransmitter release, presynaptic proteostasis, and autophagy as a significant interactor of the tau seed present in fraction 9. See Annamneedi et al. 2018; Okerlund et al., “Bassoon Controls Presynaptic Autophagy through Atg5,” (2017) Neuron 93: pp. 897-913; Waites et al., “Bassoon and Piccolo maintain synapse integrity by regulating protein ubiquitination and degradation,” (2013) EMBO J 32: pp. 954-969; Montenegro-Venegas et al., “Bassoon inhibits proteasome activity via interaction with PSMB4,” (2021) Cell Mol Life Sci 78: pp. 1545-1563. Surprisingly, missense mutations in the BSN gene have recently been identified in a family with a spatial distribution of tau pathology consistent with PSP and sporadic PSP cases. See Yabe et al., “Mutations in bassoon in individuals with familial and sporadic progressive supranuclear palsy-like syndrome,” (2018) Sci Rep 8: p. 819. Additionally, BSN expression is increased in multiple system atrophy (MSA) patients and BSN accumulates in multiple sclerosis (MS) patients. See Hashida et al., “Cloning and mapping of ZNF231, a novel brain-specific gene encoding neuronal double zinc finger protein whose expression is enhanced in a neurodegenerative disorder, multiple system atrophy (MSA),” (1998) Genomics 54: pp: 50-58; Schattling et al., “Bassoon proteinopathy drives neurodegeneration in multiple sclerosis,” (2019) Nat Neurosci 22: pp. 887-896.

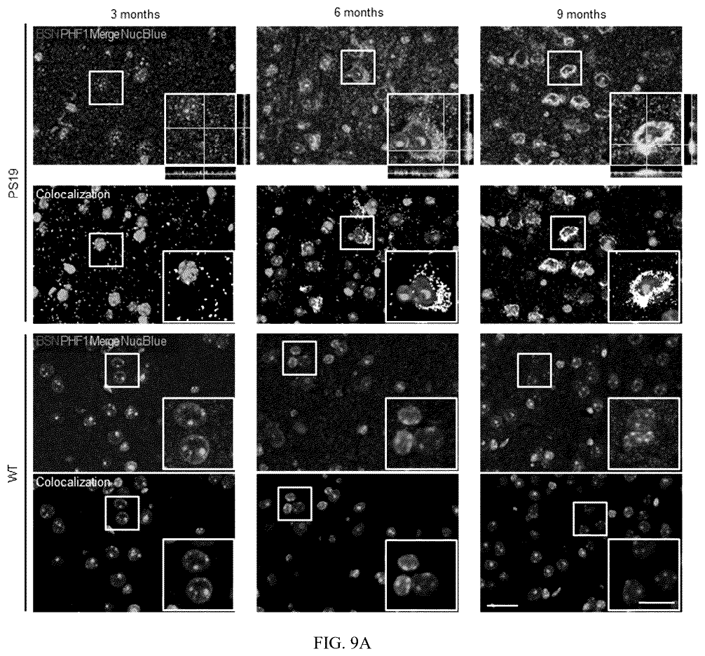

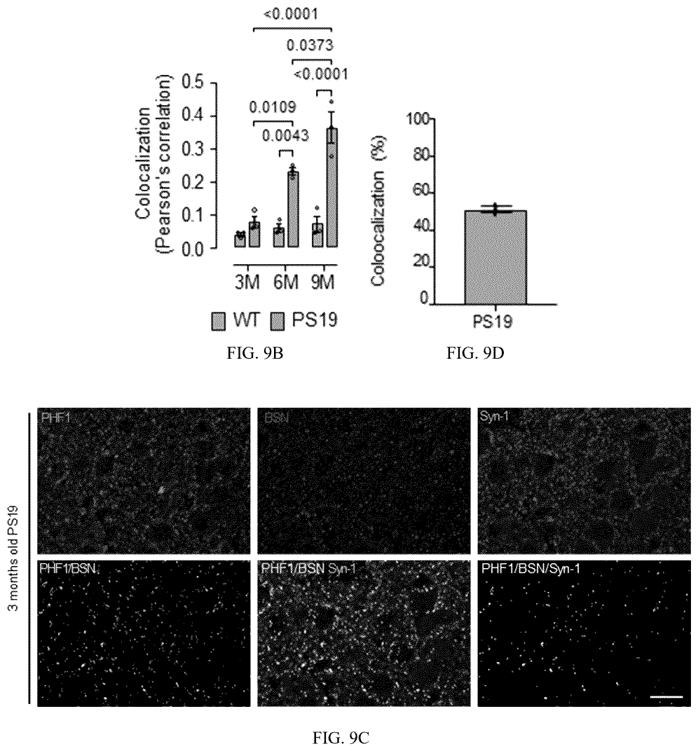

The interaction between BSN and tau seeds was confirmed by co-immunoprecipitation (co-IP) in the SEC fraction 9 from PS19 mice (FIG. 1H). Double staining also revealed a strong colocalization of BSN with tau deposits in PS19 mice (FIG. 9A-D).

FIG. 9A-D shows BSN is associated with tau pathology in PS19 mice. FIG. 9A: Representative immunofluorescence and colocalization for BSN (red), PHF1 (green), nuclei (cyan), and merge (yellow), in 3-, 6- and 9-month-old wild-type and PS19 mouse cortexes. Scale bar: 200 μm for main images and 50 μm for insets. Merge image for PS19 includes orthogonal image of reconstructed three-dimensional views. Colocalization analysis was performed to determine pixel intensity correlation between PHF1 and BSN. FIG. 9B: Pearson's correlation of BSN and PHF1 intensity in 3-, 6- and 9-month-old wild-type and PS19 mouse cortexes. FIG. 9C: 3-month-old PS19 mouse sections immunostained against PHF1, BSN, and Syn-1 (upper panels), and colocalization of PHF1 and BSN (colored yellow; lower left panel). PHF1/BSN colocalization was re-colocalized with the Syn-1 channel to obtain a final triple-colocalization between PHF1/BSN and Syn-1 (lower middle and right panels). FIG. 9D: Percentage of colocalization between PHF1/BSN puncta and Syn-1. Data are the mean±s.e.m. Experiments were performed with n=3 (FIG. 9A-D). Significance was determined by one-way ANOVA (FIG. 9B).

The degree of colocalization between BSN and phosphorylated tau increased with age (FIG. 9A, 9B). At later stages of tau pathology (9 months old), the colocalization of both signals appeared to be cytoplasmic, but at early stages (3 months old), the colocalization presented as diffuse puncta. Considering that BSN is mainly a presynaptic protein and tau has been identified at the presynapse at the early stage of pathology, the inventors performed triple staining for BSN, phospho-tau PHF1, and the presynaptic marker synapsin 1 (Syn-1) at 3 months, to show that BSN and phospho-tau colocalize at the presynaptic terminals. FIG. 9C-D; see Wu et al., “The role of pathological tau in synaptic dysfunction in Alzheimer's diseases,” (2021) Transl Neurodegener 10: p. 45; Zhou et al., “Tau association with synaptic vesicles causes presynaptic dysfunction, (2017) Nat Commun 8: pp. 15295.

These results suggest that at the early stages of pathology, the tau seed interacts with BSN at presynaptic compartments, and as the disease progresses, tau aggregates and BSN co-deposit in the cytoplasm.

### Example 2

BSN is Associated with Tau Pathology in Human Tauopathy Brain Tissue

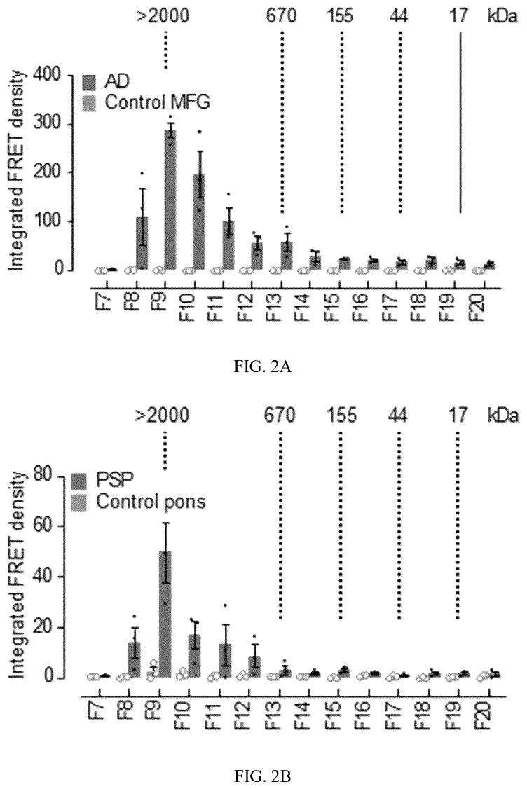

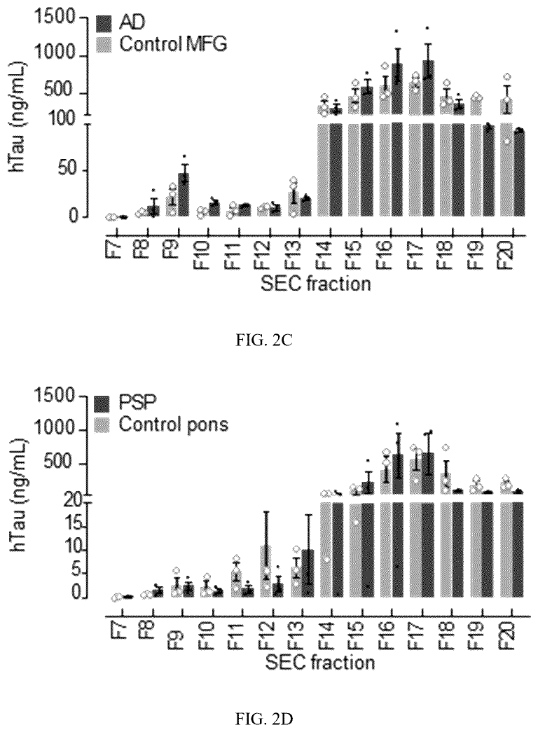

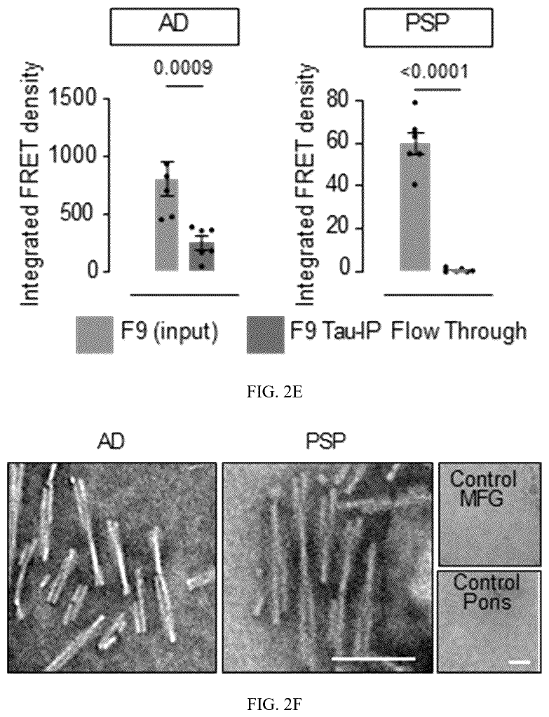

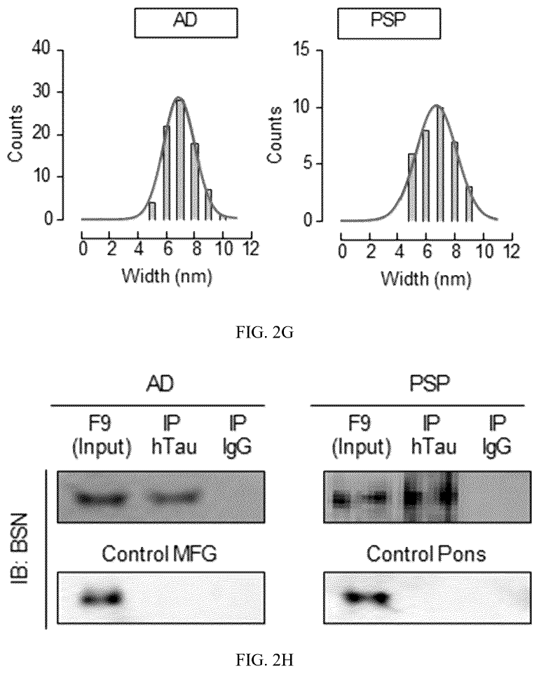

FIG. 2A-I shows that BSN is associated with tau pathology in human AD and PSP cases. FIG. 2A and FIG. 2B: Tau seeding activity of SEC fractions from human AD lysates (from middle frontal gyrus, MFG) (FIG. 2A), and PSP lysates (from pons) (FIG. 2B), compared to healthy controls from MFG and pons, respectively. FIG. 2C, FIG. 2D: Total human tau detected by ELISA in SEC fractions from human AD (FIG. 2C) and PSP (FIG. 2D) brain lysates compared to healthy controls. FIG. 2E: Tau seeding activity of the AD and PSP SEC fraction 9 (F9) containing HMW tau, before and after hTau immunoprecipitation (IP) using HT7 antibody. FIG. 2F, FIG. 2G: Tau twisted filaments present in the tau-IP product from F9 of AD and PSP SEC fractions, visualized by electron microscopy. Scale bars: 100 nm. (FIG. 2F), and width distribution (FIG. 2G). No filaments were detected in MFG and pons healthy controls. FIG. 2H, Co-IP of human tau (HT7) and BSN from SEC F9 in AD, PSP, control MFG and control pons brain lysates. FIG. 2I: Colocalization between BSN (red) and pathological phosphorylated tau species (PHF1, green) in AD and PSP brain section. Merge panel includes orthogonal image of reconstructed three-dimensional views. Colocalization analysis was performed to determine pixel intensity correlation between PHF1 and BSN. Scale bar: 10 μm. Data are shown as the mean±s.e.m. Experiments were performed with n=3 (FIG. 2A-D, F, H, I) and n=6 (FIG. 2E). significance was determined by one-way ANOVA (FIG. 2E).

The tau species with the strongest seeding activity in AD and PSP patient brains was also a HMW tau species in fraction 9 (>2,000 kDa) (FIG. 2A-B), consistent with the findings in the PS19 mice. As in the PS19 model, for both human tauopathies, tau in fraction 9 represented a small percentage of total tau in the brain (FIG. 2C-D). Interestingly, similar tau levels were detected in fraction 9 in age-matched controls; however, seeding activity was absent in these control cases (FIG. 2A-D). Depleting tau in fraction 9 from AD and PSP cases dramatically decreased seeding activity in the flow-through, confirming that a tau species in fraction 9 was responsible for seeding (FIG. 2E). EM of the tau IP material from fraction 9 revealed that in AD and PSP, the seeds are predominantly twisted filaments with widths of 6.95±1.1 nm and 6.77±1.4 nm, respectively (FIG. 2F-G). No tau filaments were detected when tau was immunoprecipitated from age-matched controls (FIG. 2F). It was then confirmed by co-IP that, in both tauopathies, BSN interacts with tau species in fraction 9 but does not interact with tau in fraction 9 from age-matched controls (FIG. 2H).

These results suggest that the interaction between BSN and tau is conformation-dependent and mainly occurs when tau forms soluble aggregates with seeding activity.

FIG. 10A and FIG. 10B shows that BSN colocalizes with a pathological tau species in human AD and PSP cases. FIG. 10A,B: Immunofluorescence, merge and colocalization images of BSN (red) and pTauS396/5404 (PHF1, green) in human AD (from middle frontal gyrus, MFG) (FIG. 10A), and PSP (from pons) (FIG. 10B), and healthy control subjects from MFG and pons, respectively. Merge image includes orthogonal image of reconstructed three-dimensional views. Scale bar: 200 μm for main images and 50 μm for insets. Experiments were performed in triplicates for AD and PSP cases.

Double staining revealed colocalization of BSN with tau deposits in AD and PSP brains (FIG. 2I; FIG. 10A, B), supporting the notion of a disease-related interaction.

### Example 3

### BSN Enhances Tau-Seeding Activity and Toxicity

Human P301S tau was overexpressed in HEK293T cells with and without human BSN to determine the effect of BSN on the pathological properties of tau.

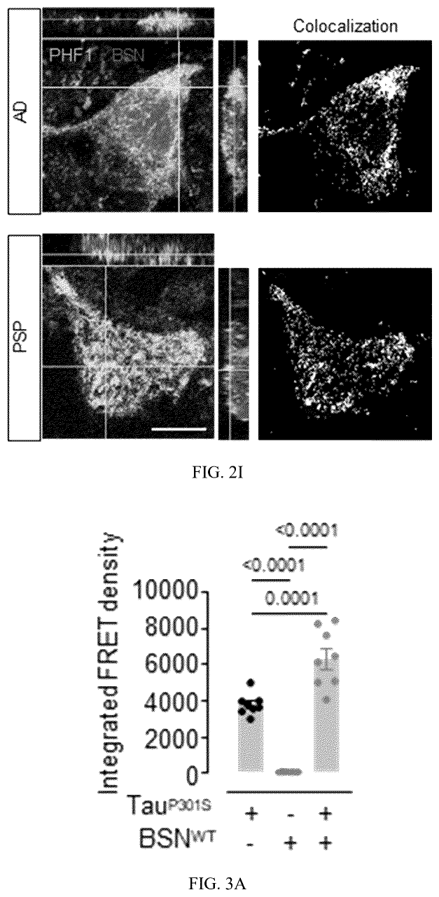

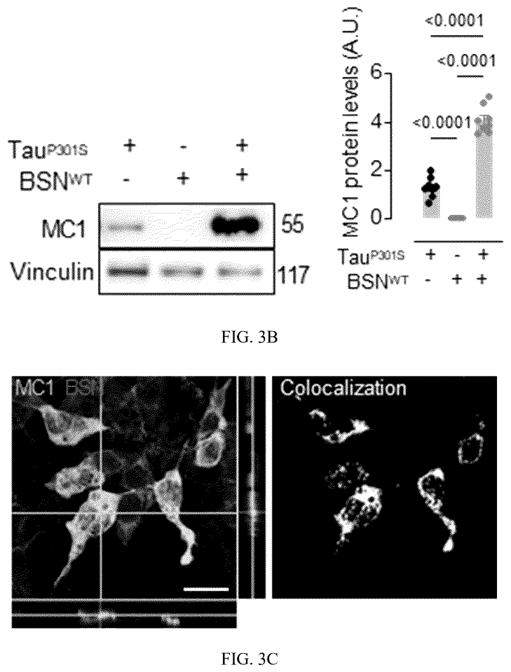

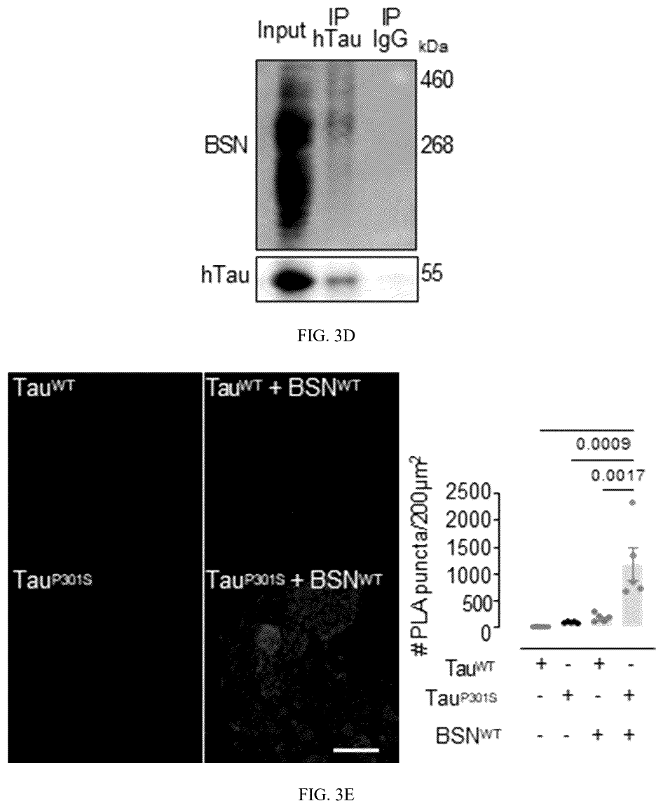

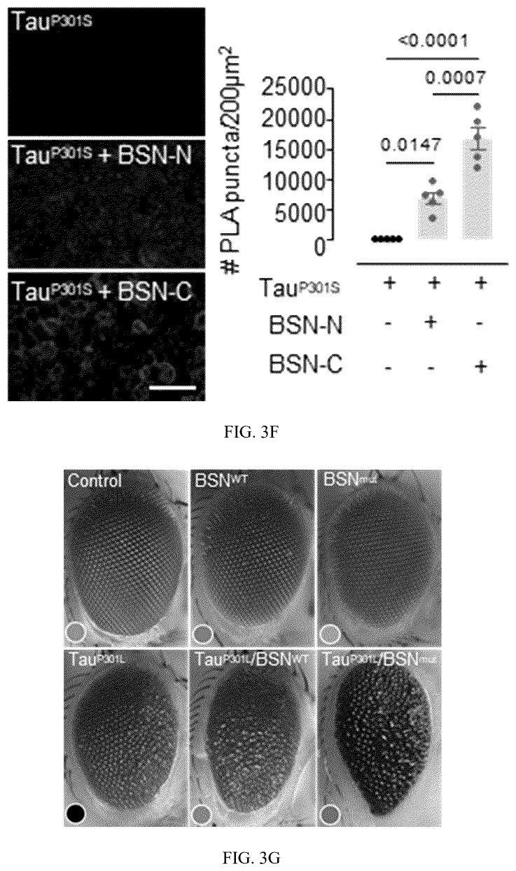

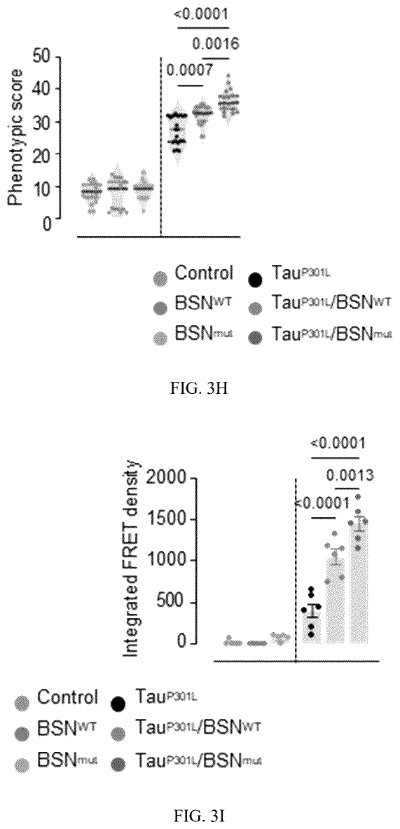

FIG. 3A-I depicts the BSN overexpression increases tau-seeding and toxicity by direct interaction. FIG. 3A: Seeding activity of HEK cell lysates expressing hTauP^{301S}, BSN^{WT}, or both plasmids. FIG. 3B: Western blot and quantification of misfolded tau levels detected with MC1 antibody in HEK cells overexpressing hTau^{P301S}, BSN^{WT} or both. FIG. 3C: Representative image of double immunofluorescences between MC1 and BSN in HEK cells overexpressing hTauP^{301S} and BSN^{WT}. Merge panel includes orthogonal image of reconstructed three-dimensional views. Colocalization analysis was performed to determine pixel intensity correlation between MC1 and BSN. Scale bar: 50 μm. FIG. 3D: Co-immunoprecipitation of hTau (HT7 antibody) and immunoblot for BSN in HEK cells overexpressing hTauP^{301S} and BSN^{WT}. FIG. 3E: PLA fluorescence paring of hTau and BSN antibodies, and quantification in HEK cells overexpressing Tau^{WT}, Tau^{WT}/BSN^{WT}, Tau^{P301S} and Ta^{P301S}/BSN^{WT}. FIG. 3F: PLA fluorescence paring of hTau and his-tag antibodies, and quantification in HEK cells overexpressing Tau^{P301S} and Tau^{P301S} plus the N- or C-terminal fragments of BSN (BSN-N or BSN-C, respectively). Scale bar: 50 μm. FIG. 3G-I Representative images (FIG. 3G), quantification of phenotypic eye degeneration (FIG. 3H), and tau-seeding activity (each point is a pool of 20 fly heads from a distinct eclosion event) (FIG. Ii) in control, BSN^{WT}, BSN^{mut}, hTau^{P301L}, hTau^{P301L}/BSN^{WT} and hTau^{P301L}/BSN^{mut} flies. Data are the mean±s.e.m. Experiments were performed with n=8 (FIG. 3A-C), n=3 (FIG. 3D), n=5 (FIG. 3E,F), n=20 (FIG. 3G,H) and n=6 (FIG. 3I). Significance was determined by one-way ANOVA (FIG. 3A, B, E, F, H, I).

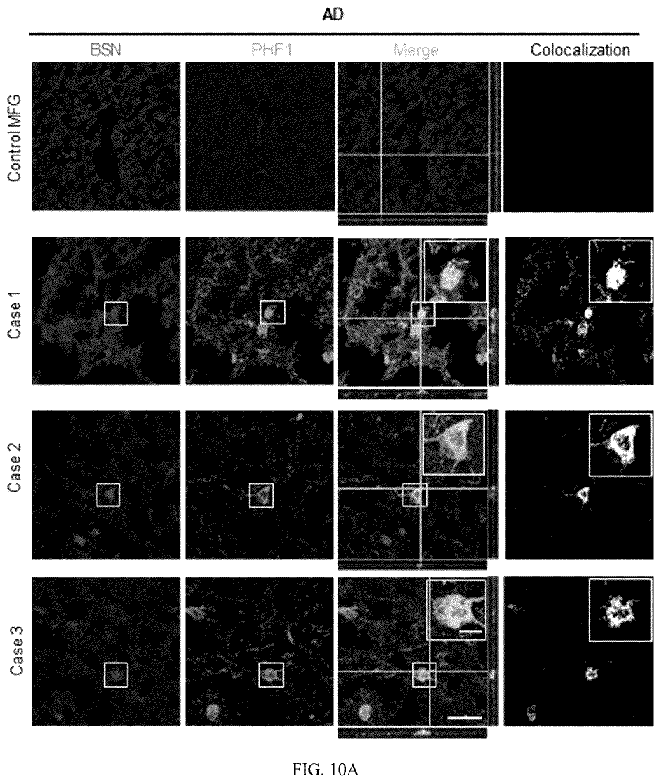

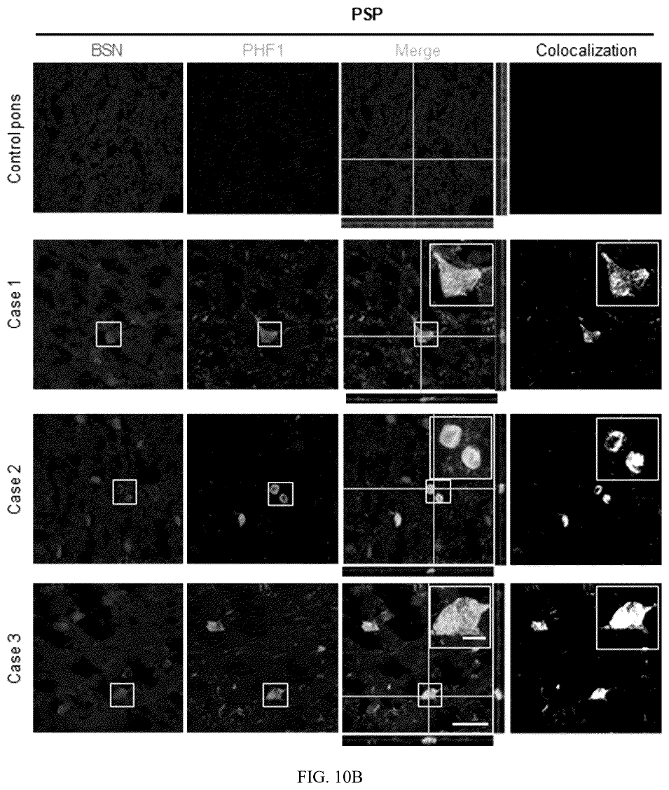

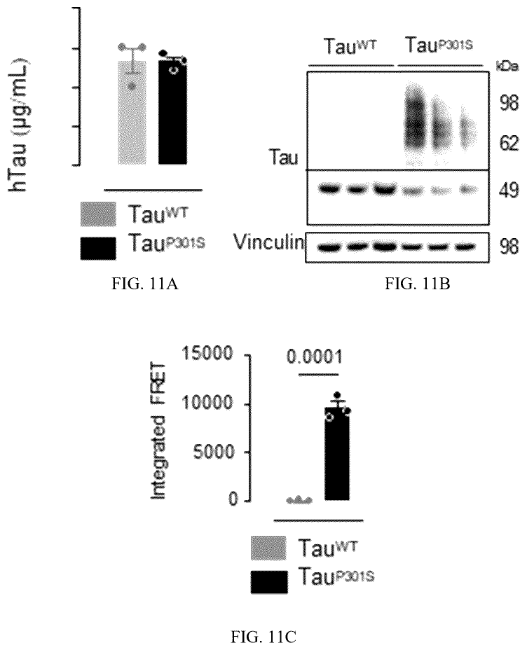

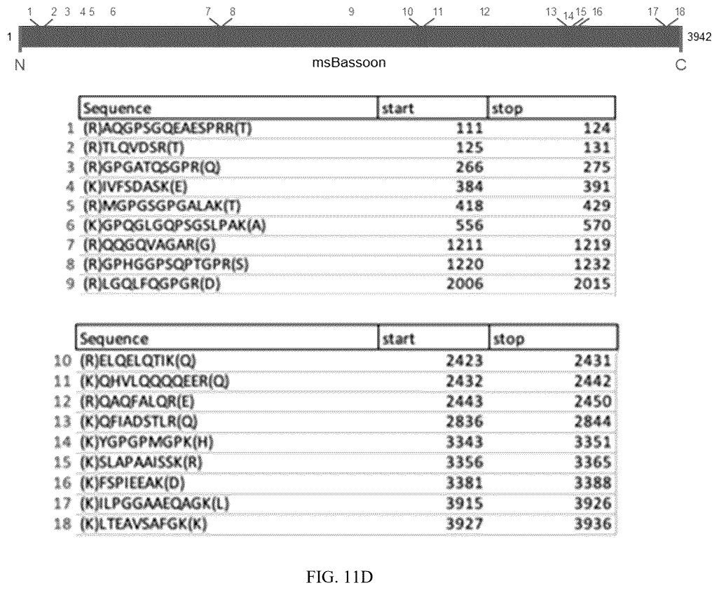

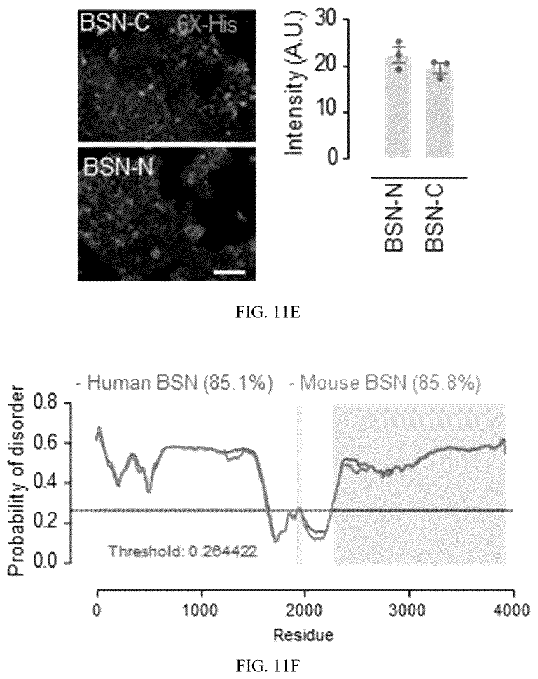

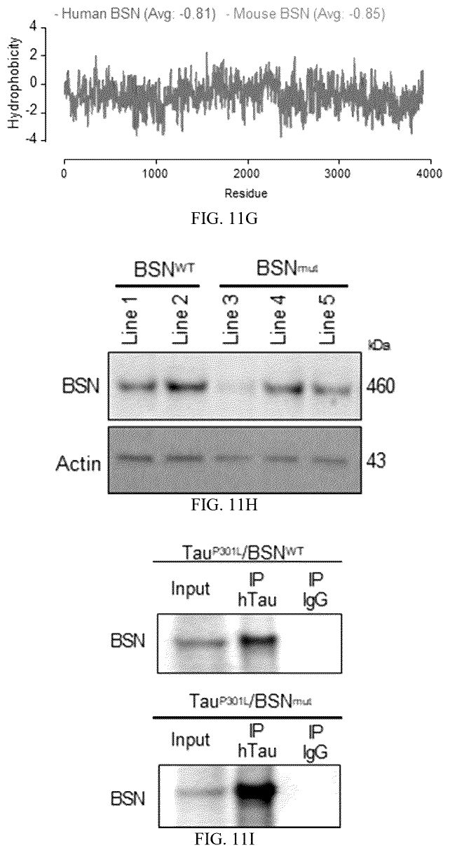

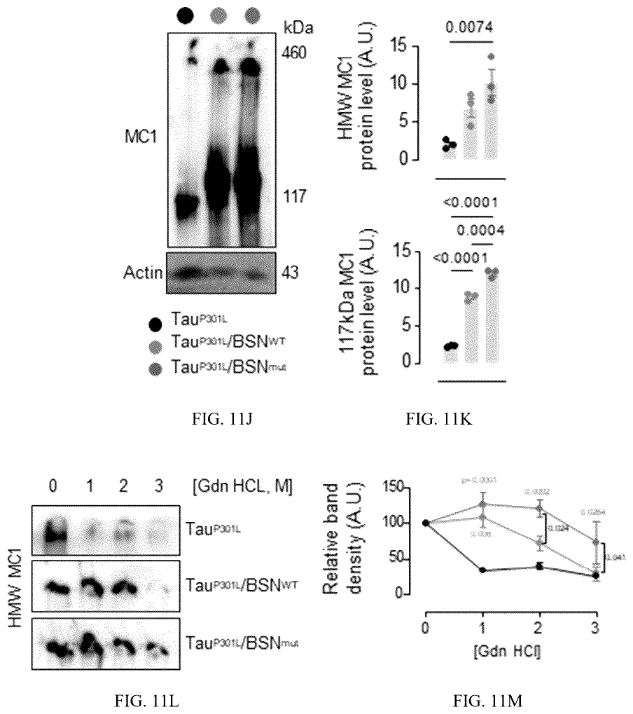

Lysates from cells overexpressing both tau and BSN demonstrated increased seeding activity compared to that observed with lysate from cells that solely overexpressed human tau (FIG. 3A). The overexpression of BSN was also associated with an increased accumulation of misfolded tau species (FIG. 3B). Double staining of HEK cells co-expressing human BSN and human P301S tau demonstrated that BSN co-deposits with misfolded tau species (FIG. 3C). In the same co-expression experiment, it was confirmed by co-IP that BSN interacts with tau (FIG. 3D). The inventors performed a proximity ligation assay (PLA) on HEK cells co-expressing BSN and human P301S tau to determine whether BSN interacts directly with tau. A strong PLA signal in cells stained for human tau and BSN antibodies was observed, indicating that human P301S tau and BSN are within interaction proximity (<40 nm) (FIG. 3E). Considering that BSN interacts with the tau seed but not monomeric tau from PS19 mice (FIG. 1F-G) and with fraction 9 tau from AD and PSP patients but not that from age match controls without seeding activity (FIG. 2H), the inventors aimed to determine whether this interaction is conformation-dependent. Since overexpressing a similar amount of human wild-type tau does not form stable aggregates and does not show seeding activity as human P301S tau (FIG. 11A-C), PLA on HEK cells co-expressing BSN and human wild-type tau was performed to determine if BSN interacts with non-aggregating tau. Minimal PLA signal was observed in cells stained for human tau and BSN antibodies (FIG. 3E), demonstrating that BSN has a higher affinity for tau when tau adopts a misfolded or aggregated conformation.

FIG. 11A-M shows BSN overexpression increases tau accumulation in vitro and in a Drosophila model of tauopathy. FIG. 11A-C: hTau levels by ELISA (FIG. 11A), Western blot of total tau (FIG. 11B), seeding activity (FIG. 11C) from HEK cells overexpressing hTau^{WT} or hTau^{P301S}. Western blot membrane was overexposed in the upper level for better visualization of oligomeric tau (denoted by the asterisk*). FIG. 11D: Location of BSN peptides detected by IP-mass spec. FIG. 11E: Representative images, and quantification of 6X-His immunofluorescences from HEK cells overexpressing Tau^{P301S} and 6X-His-BSN N- or C-terminal fragments (BSN-N or BSN-C, respectively). FIG. 11F: Disordered residues of human and mouse bassoon. 85.1% of residues in human BSN and 85.8% of residues in mouse BSN were predicted to be disordered. FIG. 11F: Hydrophobicity profile of bassoon using method of Kyte & Doolittle. Average hydrophobicity is −0.81 for human BSN, and −0.85 mouse BSN. FIG. 11H: Western blot confirming the overexpression of the UAS-BSN under the GMR-Gal4 driver in different fly lines. FIG. 11I: Western blot for BSN of co-immunoprecipitation (Co-IP) of human tau (HT7) from hTau^{P301L}/BSN^{WT} and hTau^{P301L}/BSN^{mut} fly head lysates. FIG. 11J,K: Western blot (FIG. 11J) of the level of HMW and 117 kDa MC1 detected and quantified (FIG. 11K) in Tau^{P301L}, Tau^{P301L}/BSN^{WT} and Ta^{P301L}/BSN^{mut} fly head lysates. Each sample is a pool of 20 fly brains from 3 different eclosion events. FIG. 11L,M Western blot (FIG. 11L) and quantification (FIG. 11M) of misfolded tau (MC1) in fly lysates denatured by increasing concentrations of guanidine HCl, in hTau^{P301L}, hTau^{P301L}/BSN^{WT} and hTau^{P301L}/BSN^{mut} head lysates. Data are the mean±s.e.m. Experiments were performed with n=3 (FIG. 11A-C, E, J-M). Significance was determined by unpaired Student's t-test (FIG. 11B-E) and one-way ANOVA (FIG. 11K, M).

In the tau-seed IP-MS analysis (FIG. 1F), BSN peptides were identified corresponding to the N-terminal and the C-terminal regions of the BSN protein (FIG. 11D), suggesting that the tau seed interacts with full-length BSN. The inventors co-expressed human P301S tau with the N-terminal fragment (1-850) or with the C-terminal region (2450-3942) fused to 6×His-tag of human BSN in HEK cells, with both fragments expressed at a similar level (FIG. 11E), to determine the region of BSN that interacts with aggregated tau. PLA was performed in these cells using anti-human tau and anti-his-tag antibodies. PLA signals were observed in HEK cells co-transfected with P301S tau and BSN C-terminal fragment (FIG. 3F), suggesting that BSN could interact with tau aggregates through its C-terminal region. Nevertheless, although significantly lower, PLA signals were observed in HEK cells co-transfected with P301S tau and BSN N-terminal fragment (FIG. 3F), suggesting that BSN could also interact with tau aggregates via its N-terminal region. Interestingly, it has been shown that large proteins with lower-than-average hydrophobicity, more intrinsically disordered residues and longer regions of disorder are more susceptible to aberrant interactions with amyloid-like aggregates. See Olzscha et al., “Amyloid-like aggregates sequester numerous metastable proteins with essential cellular functions,” (2011) Cell 144: pp. 67-78. Considering that BSN is a large protein that is almost entirely disordered (FIG. 11F and Shattling et al. 2019) and has a lower-than-average hydrophobicity (FIG. 11G), BSN has the potential to engage in widespread aberrant interactions with aggregated forms of tau.

The inventors further overexpressed human BSN in two transgenic Drosophila melanogaster lines to investigate the effects of BSN upregulation in vivo, one line overexpressing wild-type BSN and the other overexpressing BSN harboring the P3866A mutation recently identified in patients with a spatial distribution of tau pathology consistent with PSP (FIG. 11H; Yabe et al. 2018). Neither of these lines developed a degenerative eye phenotype (FIG. 3G). However, the overexpression of wild-type BSN enhanced the degenerative eye phenotype in the Drosophila model expressing human tau with the P301L mutation (hTau-P301L) by increasing the disruption of the ommatidial structure (FIG. 3G-H). The degenerative eye phenotype was intensified in the hTau-P301L fly when the BSN mutant was overexpressed (FIG. 3G-H), suggesting that BSN exacerbates tau toxicity in vivo. The inventors confirmed by co-IP that wild-type and mutant BSN also interact with tau in flies (FIG. 11I). Western blotting showed that both wild-type and mutant BSN promote the accumulation of the misfolded tau species, as detected by MC1 antibody (FIG. 11J-K). A guanidine stability assay was then used to further examine stability differences between fly hTau-P301L aggregates. In the presence of wild-type and mutant BSN, tau aggregates were significantly more resistant to disaggregation by guanidine hydrochloride (FIG. 11L-M). As observed in the cellular model, BSN overexpression in hTau-P301L flies lead to an increase in tau-seeding activity, which was even higher with mutant BSN (FIG. 3I).

These results suggest that BSN could facilitates tau-related toxicity by enhancing the formation and stabilization of pathological tau species exhibiting seeding activity.

### Example 4

### BSN Knockdown Reduces Tau Spread In Vivo

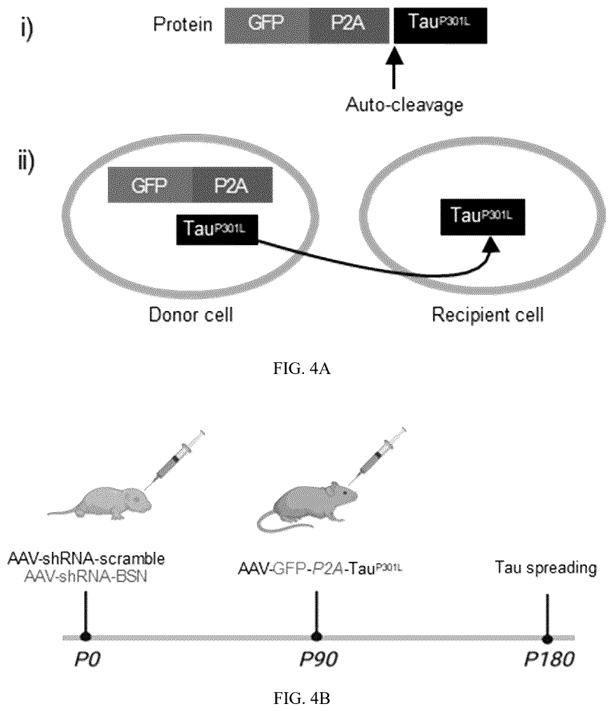

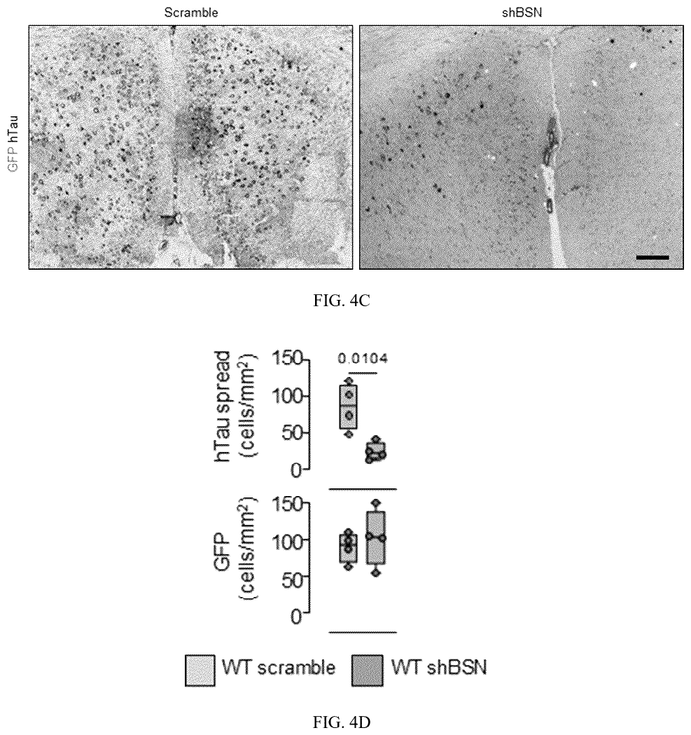

The next aim was to investigate whether BSN is critical for tau spreading in the brain using a well-characterized adeno-associated virus (AAV)-based spreading model. In this model, GFP and hTau^{P301L} proteins are translated from mRNA-GFP-P2A-hTau^{P301L} under the control of the cytomegalovirus promoter. See Wegmann et al., “Experimental evidence for the age dependence of tau protein spread in the brain,” (2019) Sci Adv 5: eaaw6404. This model allows for discrimination between transduced neurons expressing both GFP and hTau^{P301L} from those expressing only hTau^{P301L} due to spreading (FIG. 4A).

FIG. 4A-D shows that BSN contributes to tau-spreading in vivo. FIG. 4A: Schematic of the GFP-P2A-hTau^{P301L} tau-spreading mouse model. GFP-positive neurons are hTau-donors, spreading hTau^{P301L} to recipient GFP-negative neurons. FIG. 4B: Timeline of injections in wild-type mice. These animals were injected with scramble or shBSN AAV at P0, then with AAV-GFP-P2A-hTau^{P301L} at P90. Animals were euthanized at P180 to evaluate hTau spreading. FIG. 4C, Representative immunofluorescences for hTau (HT7 antibody) in animals overexpressing GFP-P2A-hTau^{P301L} injected with scramble shRNA (left) and BSN shRNA (right). Scale bar: 100 μm. FIG. 4D, Quantification of cortical hTau⁺/GFP⁻ cells (top) and GFP⁺ cells (bottom) per mm². Values are given as the means±s.e.m. Experiments were performed with n=4, and significance was determined by unpaired Student t-test.

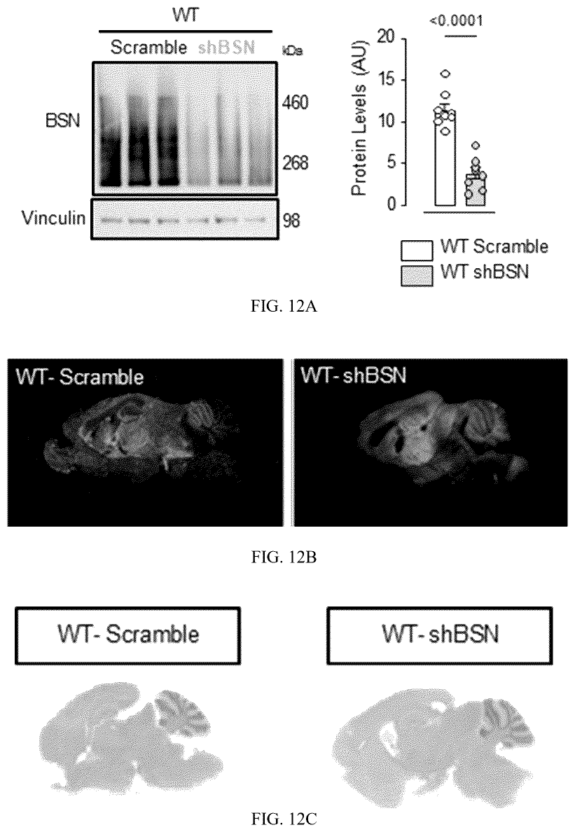

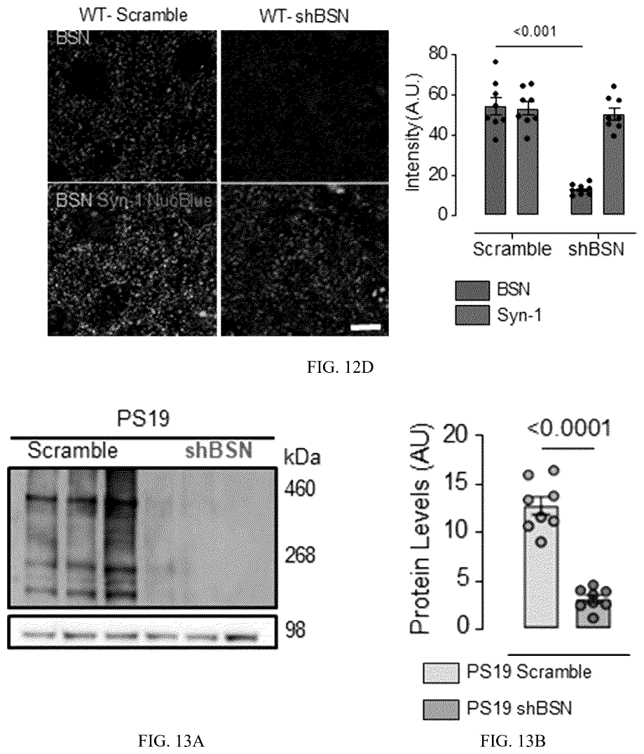

The inventors performed neonatal (P0) intracerebroventricular (ICV) injection of an AAV harboring a short-hairpin RNA (shRNA) against murine Bsn (shBSN) or control ‘scramble’ shRNA in wild-type mice to downregulate the expression of Bsn in parallel. shBSN significantly downregulated Bsn (to ˜60%) expression in vivo in wild-type mice (FIG. 12A).

FIG. 12A-D shows that murine BSN downregulation does not produce gross brain abnormalities. FIG. 12A: Western blot and quantification of BSN downregulation in WT mice. FIG. 12B: Detection of BFP2 reporter in AAV scramble and AAV shBSN mice, confirming the widespread expression of both sequences. FIG. 12C: H&E staining of WT mice injected with scramble and shBSN shRNA. FIG. 12D: BSN and Syn-1 immunofluoresce in WT mouse cortexes injected scramble and shBSN shRNA, and mean intensity of BSN and Syn-1. Data are shown as the mean±s.e.m. Experiments were performed with n=8 (FIG. 12A-D). Significance was determined by unpaired Student's t-test (FIG. 12A) and one-way ANOVA (FIG. 12D).

Both scramble and shBSN shRNAs encode for a blue fluorescent protein (BFP2) reporter, allowing the visualization of AAV transduction throughout the brain without producing gross abnormalities in brain sections or negatively affecting presynaptic integrity (FIG. 12B-D). Specifically, neonatal (P0) mice received ICV injections of pAAV9-mTagBFP2-U6-mBsn-shRNA or control pAAV9-mTagBFP2-U6-Scr-shRNA, and three months later, the inventors administered stereotaxic injections of the pAAV-GFP-(P2A)-hTau^{P301L} into the cortex and hippocampus. Three months post-injection, the mice were euthanized, and tau spread was assessed by immunostaining using the anti-hTau antibody HT7 (FIG. 4B, C). Tau spreading was quantified in both groups by counting the number of hTau⁺/GFP⁻ cells per mm² (FIG. 4C, D).

The inventors observed substantial tau spreading in mice injected with scramble AAV, whereas spreading was significantly reduced in BSN knockdown mice (FIG. 4D). This decrease was not due to differences in the number of transduced cells, as a comparable number of GFP+ cells was observed in both groups (FIG. 4D).

### Example 5

### BSN Downregulation Reduced Pathology and Seed Stability In Vivo

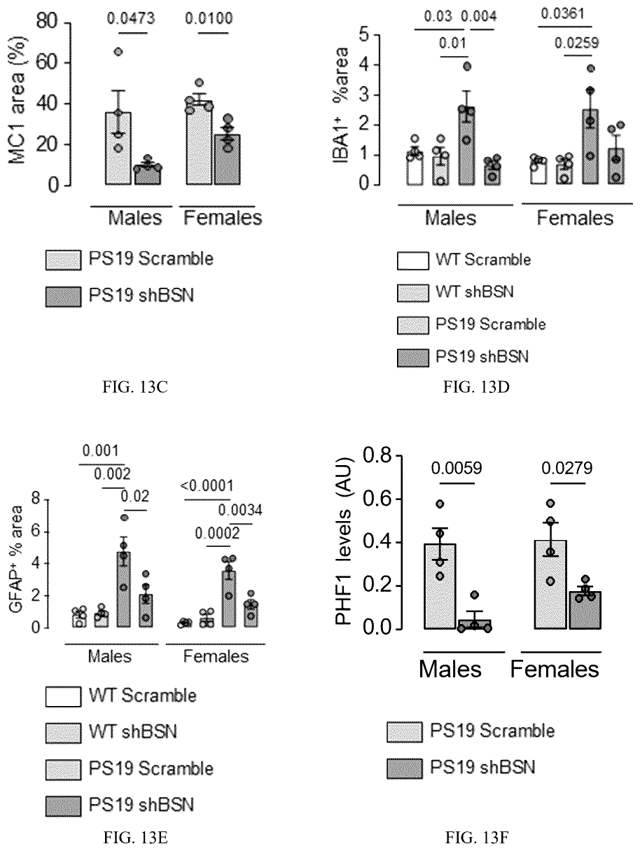

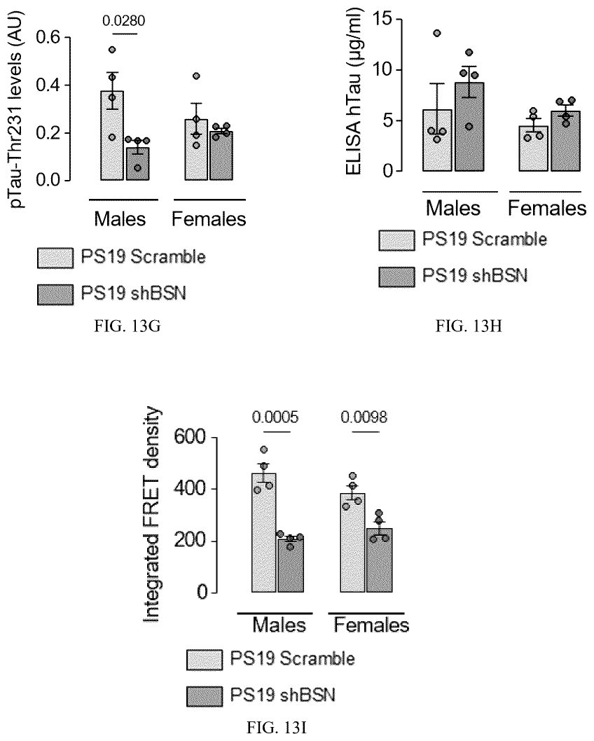

It was next investigated whether BSN downregulation affects tau pathology and associated pathogenesis in PS19 mice. Neonatal PS19 and wild-type littermates were injected with AAV coding scramble or BSN shRNA. Four months post-injection, the mice were sacrificed, and western blotting was conducted to confirm BSN downregulation (FIG. 13A-B).

FIG. 13A-I shows reducing BSN levels mitigate tau pathology in male and female PS19 mice. FIG. 13A, B: Western blot (FIG. 13A) and quantification (FIG. 13B) of BSN downregulation in PS19 mice. FIG. 13C: Quantification of MC1 immunostaining in shBSN and scramble, of PS19 mice by sex as percentage of area. FIG. 13D, E: Quantification of hippocampal immunofluorescence of GFAP (FIG. 13D) and IBA1 (FIG. 13E) in WT_{scramble}, WT_{shBSN}, PS19_{scramble} and PS19_{shBSN} mice, separated by sex. FIG. 13F, G: Western blot quantification of pTauS396/5404 (PHF1) (FIG. 13F) and pTau Thr231 (FIG. 13G) in shBSN and scramble PS19 mouse brain lysate, separate by sex. FIG. 13H, I: Total human tau levels by ELISA (FIG. 13H) and tau-seeding activity (FIG. 13I) in shBSN and scrambled PS19 mouse brain lysates, separated by sex. Data are shown as the mean±s.e.m. Experiments were performed with n=8 (FIG. 13A, B) and n=4 (FIG. 13C-I). Significance was determined by unpaired Student's t-test (FIG. 13B, C, F-I) and one-way ANOVA (FIG. 13D, E).

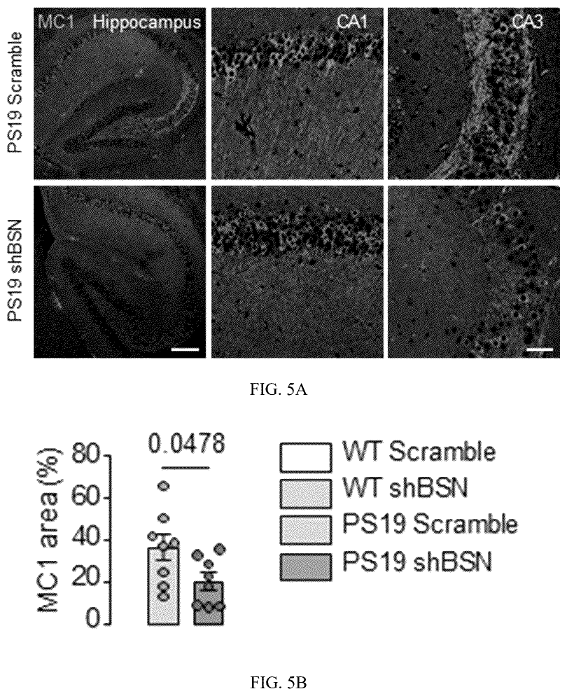

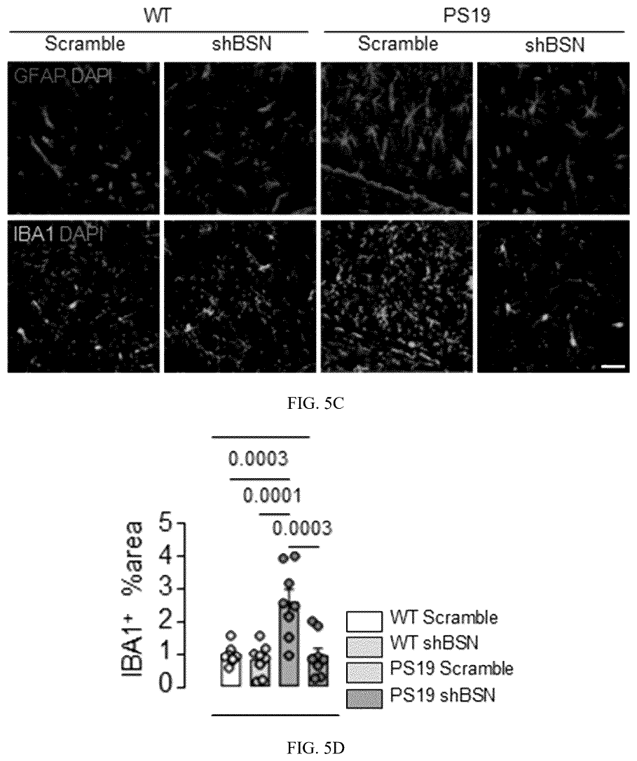

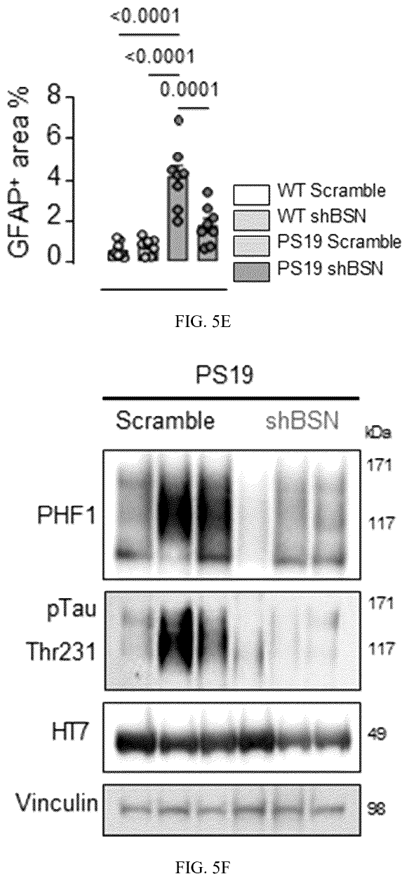

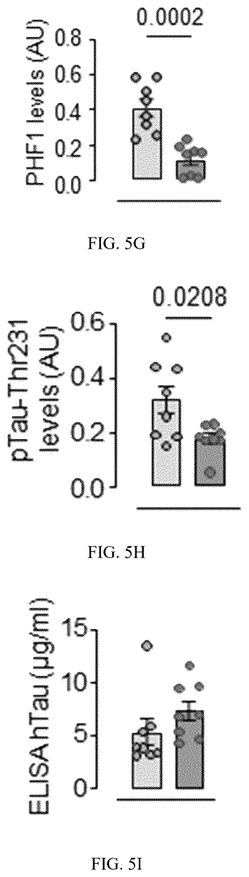

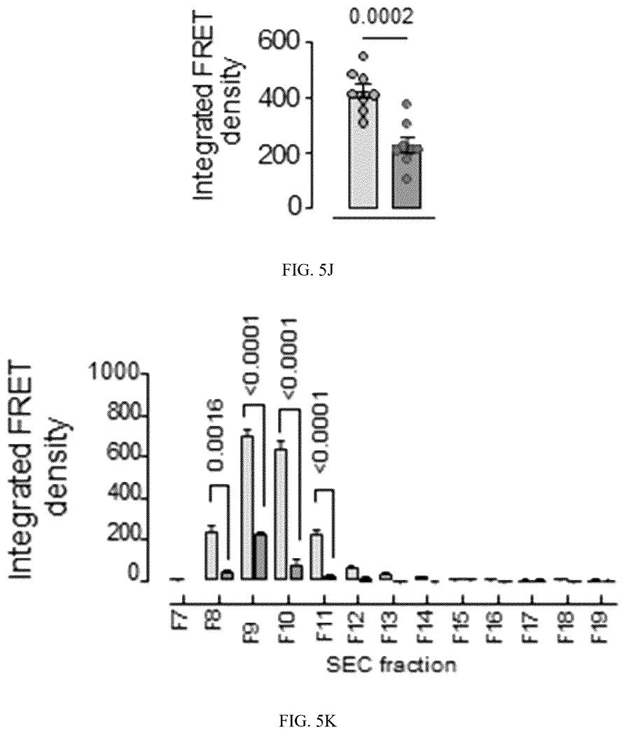

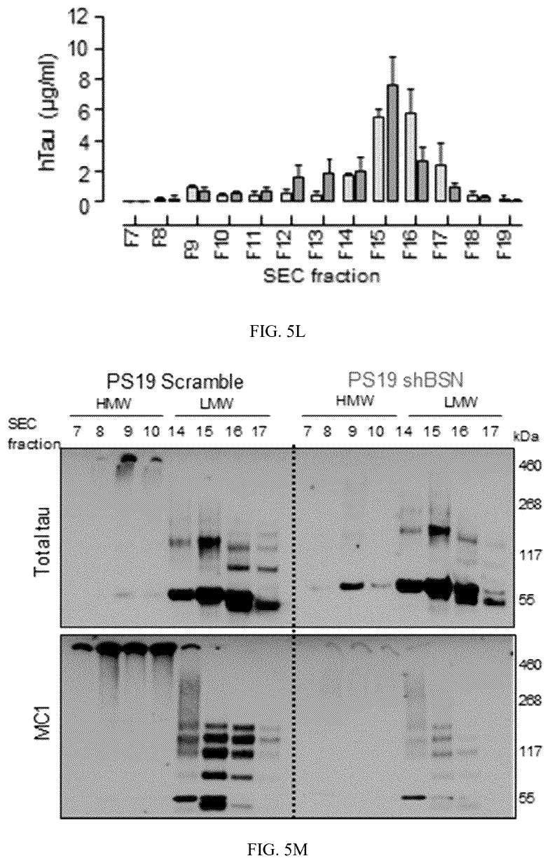

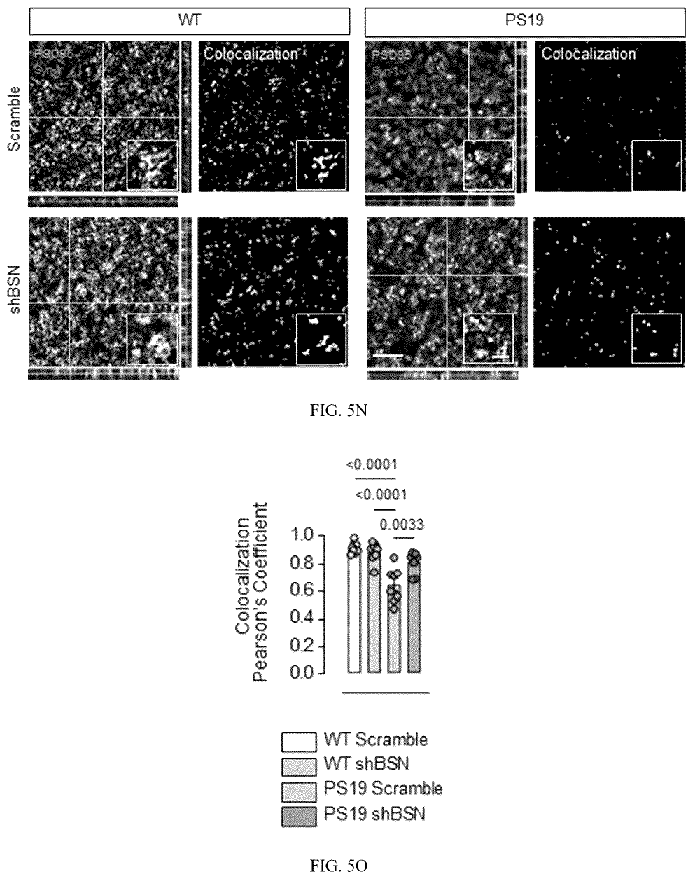

FIG. 5A-O shows that BSN downregulation reduces tau pathology and tau-seeding stability in vivo. FIG. 5A: Representative images of hippocampal MC1 immunostaining in 4-month-old PS19 mice injected with shBSN and scramble shRNA. Scale bar: 200 μm, and 50 μm for CA1 and CA3 insets. FIG. 5B: Quantification of MC1 immunostaining in shBSN and scramble PS19 mice as percentage of area. FIG. 5C-E: Hippocampal immunofluorescence (FIG. 5C) using specific antibodies against GFAP (red) and IBA1 (green) in WT_{scramble}, WT_{shBSN}, PS19_{scramble} and PS19_{shBSN} mice. Quantification was performed as percentage of area for IBA1 (FIG. 5D) and GFAP (FIG. 5E) positive cells. FIG. 5F-H: Western blot (FIG. 5F) and quantification of specific antibodies against pTauS396/5404 (PHF1) (FIG. 5G) and pTau Thr231 (FIG. 5H) in shBSN and scramble PS19 mouse brain. FIG. 5I, FIG. 5J: Total human tau levels measured by ELISA (FIG. 5I) and tau-seeding activity (FIG. 5J) in shBSN and scrambled shRNA PS19 mouse brain lysates. FIG. 5K, FIG. 5L: Seeding activity (FIG. 5K), and total hTau levels (FIG. 5L) in SEC fractions from shBSN or scramble PS19 mouse brain lysates. FIG. 5M: Western blot of total tau (HT7, top blot) and misfolded tau (MC1, bottom blot) in SEC fractions (fractions 7 to 10, HMW; fractions 14 to 17, LMW) from shBSN and scramble PS19 mouse brain lysates. FIG. 5N: Representative merge images of immunofluorescence of PSD95 (green) and Syn-1 (magenta). Merge panel includes orthogonal image of reconstructed three-dimensional views. Colocalization analysis was performed to determine pixel intensity correlation between PSD95 and Syn-1. FIG. 5O: Colocalization quantification by Pearson's correlation coefficient. Data are the mean±s.e.m. Experiments were performed with n=8 (FIG. 5A-J, N, O), n=6 (FIG. 5K, L), and n=3 (FIG. 5M). Significance was determined by unpaired Student's t-test (FIG. 5B, G-J) and one-way ANOVA (FIG. 5D, E, O).

Detection of the anti-misfolded tau antibody (MC1) revealed that decreasing BSN levels reduced tau pathology in the hippocampus of PS19 mice (FIG. 5A-B). Astrogliosis and microgliosis were similarly decreased (FIG. 5C-E). The effect of BSN downregulation on tau pathology and gliosis was significant in both female and male PS19 mice (FIG. 13C-E). Western blotting in non-reducing conditions revealed that Bsn downregulation decreased phosphorylated tau aggregates (p-tau; detected using PHF1 (pS396/S404) and pThr231 antibodies) in the total lysate (FIG. 5F-H). ELISA revealed that BSN downregulation did not influence total human tau levels in PS19 brains (FIG. 5I); however, a dramatic decrease in tau-seeding activity was observed (FIG. 5J). Bsn downregulation significantly decreased the levels of PHF1 aggregates, and seeding activity in females and males, but the effect on pThr231 tau levels was solely observed in male PS19 mice (FIG. 13F-I). The inventors then performed size exclusion chromatography on the brain lysates to determine if BSN downregulation affects HMW tau-seed activity. As expected, the seeding activity of the fraction containing the HMW tau seed (fraction 9) was significantly lower in PS19_{shBSN} than in PS19_{scramble} samples (FIG. 5K). Interestingly, a similar distribution of total tau levels was observed in the SEC fractions of PS19_{shBSN} and PS19_{scramble} samples (FIG. 5L), suggesting that BSN plays a minimal role in the formation of HMW tau complexes but instead affects the properties of the tau seed by enhancing its seeding activity, perhaps by stabilizing and promoting HMW tau-seed aggregation. SEC fractions were analyzed by western blot with strong reducing conditions to test this hypothesis. Tau remained at the top of the gel in the HMW fractions (F8, F9, and F10) in samples from PS19_{scramble}, but in samples from PS19_{shBSN}, HMW tau was resolved in the gel as a monomer (FIG. 5M, top blot). When using the MC1 antibody, tau again remained at the top of the gel in HMW SEC fractions in PS19_{scramble} samples, but in PS19_{shBSN} samples, no misfolded tau was observed in the HMW SEC fractions in strong reducing conditions (FIG. 5M, bottom blot). These data suggest that BSN stabilizes HMW tau aggregates, increasing their resistance to degradation in reducing conditions.

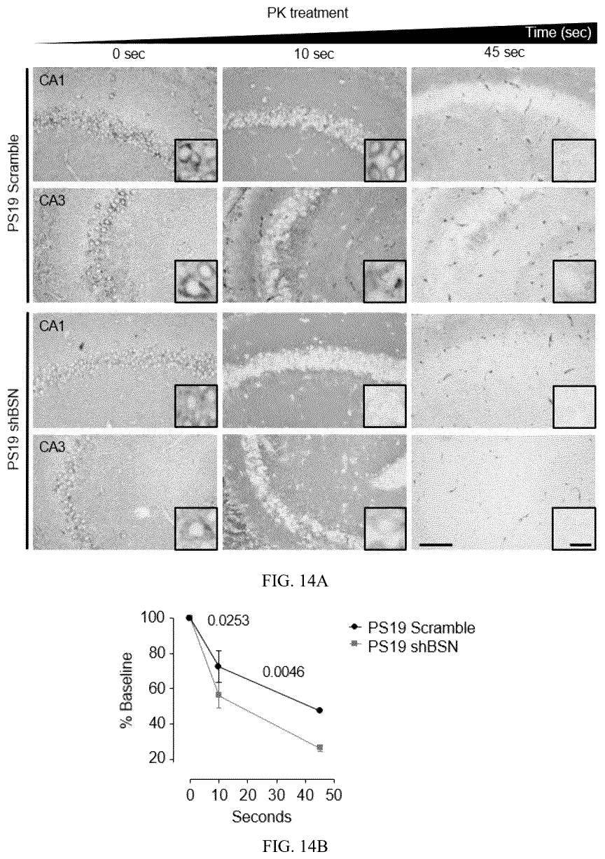

The inventors next assessed the proteinase K (PK) sensitivity of misfolded tau in brain sections from PS19_{scramble} and PS19_{shBSN} mice to further explore if BSN affects the structural properties of tau aggregates. Misfolded tau detected by MC1 in PS19_{shBSN} samples was considerably more sensitive to protease degradation, with the intensity dramatically decreased after 45 sec of PK digestion (FIG. 14A,B).

FIG. 14A, B show the increased susceptibility to PK degradation of misfolded tau aggregates after BSN downregulation in PS19 mice. FIG. 14A, B: Proteinase K sensitivity assay (FIG. 14A) and quantification (FIG. 14B) of MC1 IHC intensity at 0, 10 and 45 sec after PK treatment in scramble and shBSN PS19 brain sections. Scale bar: 100 μm for main pictures and 10μ for insets. Data are shown as the mean±s.e.m. Experiments were performed with n=3 (FIG. 14A, B). Significance was determined by unpaired Student's t-test (FIG. 14B).

This result suggests that in the absence of BSN, tau aggregates have a relaxed structure and are accessible for digestion, whereas in the presence of BSN, tau aggregates are more compacted and PK-resistant.

Since synaptic loss occurs as early as 3 months of age in PS19 mice, (Yoshiyama et al. 2007), the inventors assessed whether the decrease in tau pathology after BSN downregulation corresponded to improved synaptic integrity in 4-month-old PS19 mice. Quantification of the colocalization of presynaptic (synapsin-1) and postsynaptic (PSD-95) marker puncta in the cortex of PS19_{scramble} and PS19_{shBSN} mice (FIG. 5N) revealed a significant improvement in synaptic integrity in PS19 mice in which Bsn was downregulated (FIG. 5O) in both females and males (FIG. 15A).

FIG. 15A-C show the rescue of synaptic integrity in PS19 after BSN downregulation. FIG. 15A: Pearson's correlation of PSD95 and Syn-1 in WT_{scramble}, WT_{shBSN}, PS19_{scramble} and PS19_{shBSN} mice, separated by sex. FIG. 15B: Representative immunofluorescence in scramble and shBSN PS19 mice for misfolded tau (MC1), and Syn-1 protein. Colocalization analysis was performed to determine pixel intensity correlation between MC1 and Syn-1. Scale bar: 50 μm for main panels and 10 μm for insets. FIG. 15C: Pearson's correlation of MC1 and Syn-1 immunostainings in scramble and shBSN PS19 mice. Data are shown as the mean±s.e.m. Experiments were performed with n=4 (FIG. 15A) and n=8 (FIG. 15B, C). Significance was determined by one-way ANOVA (FIG. 15A) and unpaired Student's t-test (FIG. 15C).

The effect of BSN downregulation on synaptic integrity in PS19 mice could be due to a decrease in the accumulation of misfolded tau at the presynapse (FIG. 15B-C).

### Example 6

### Reducing BSN Rescues the Phenotypes in a Tauopathy Mouse Model

A second cohort of 6-month-old PS19 and wild-type littermates were injected at P0 with scramble shRNA or BSN shRNA to determine if this synaptic integrity rescue represented a functional improvement. The inventors measured long-term potentiation (LTP) in the hippocampal area CA1 in mouse brain slices. Field excitatory postsynaptic potentials (fEPSPs) were monitored that were evoked by extracellular stimulation of the Schaffer collateral pathway and induced LTP using high-frequency stimulation (four stimuli of 100 Hz for 1 sec with a 10-sec inter-stimulus interval). Severely impaired hippocampal LTP was observed in PS19_{scramble} vs. WT_{scramble} and WT_{shBSN} mice.

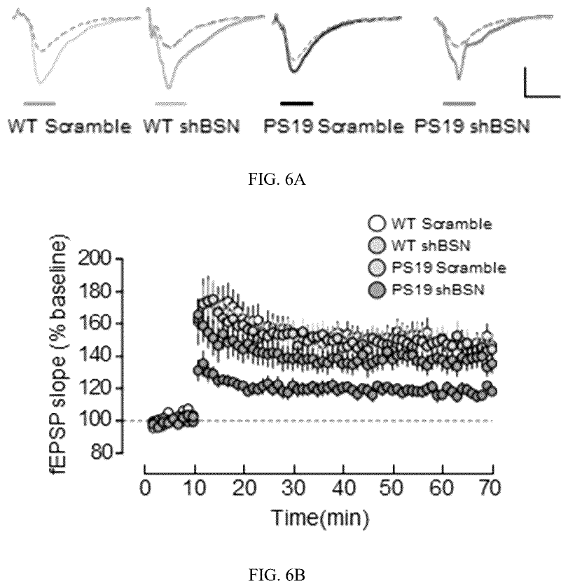

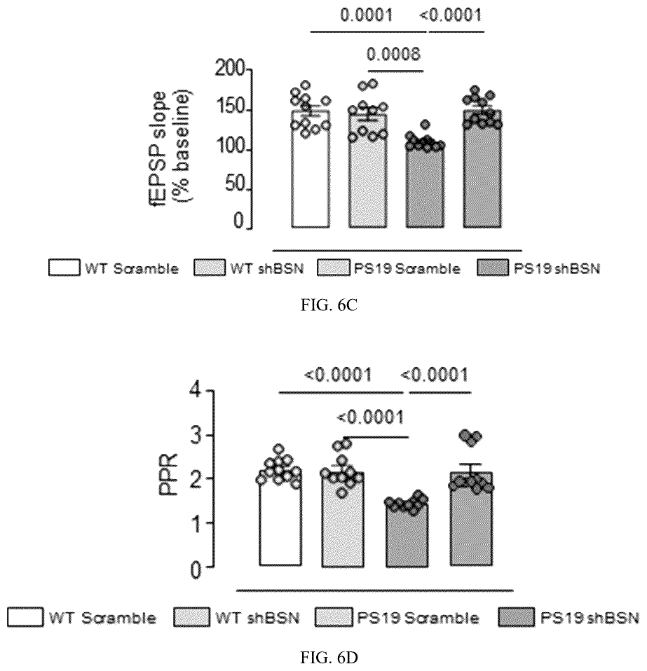

FIG. 6A-D shows BSN downregulation restores electrophysiological impairments in PS19 mice. FIG. 6A: Superimposed fEPSP traces produced by stimulation of the Schaffer collateral pathway and recorded in CA1 brain slices before (gray dotted lines) and after (solid lines) LTP induction. Vertical bar: 300 mV, Horizontal bar: 10 ms. FIG. 6B-D: Summary of in vivo LTP (FIG. 6B), averaged from minute 60 to 70 (FIG. 6C), and paired-pulse ratio (PPR) (FIG. 6D) in 6-month-old WT and PS19 mice injected with shBSN or scramble shRNA. Data represent the mean±s.e.m. Experiments were performed with n=11 for WT_{scramble}, n=11 for PS19_{scramble}, n=10 for WT_{shBSN}, and n=11 for PS19_{shBSN} mice (FIG. 6B-D). Significance was determined by two-way ANOVA (FIG. 6C, D).

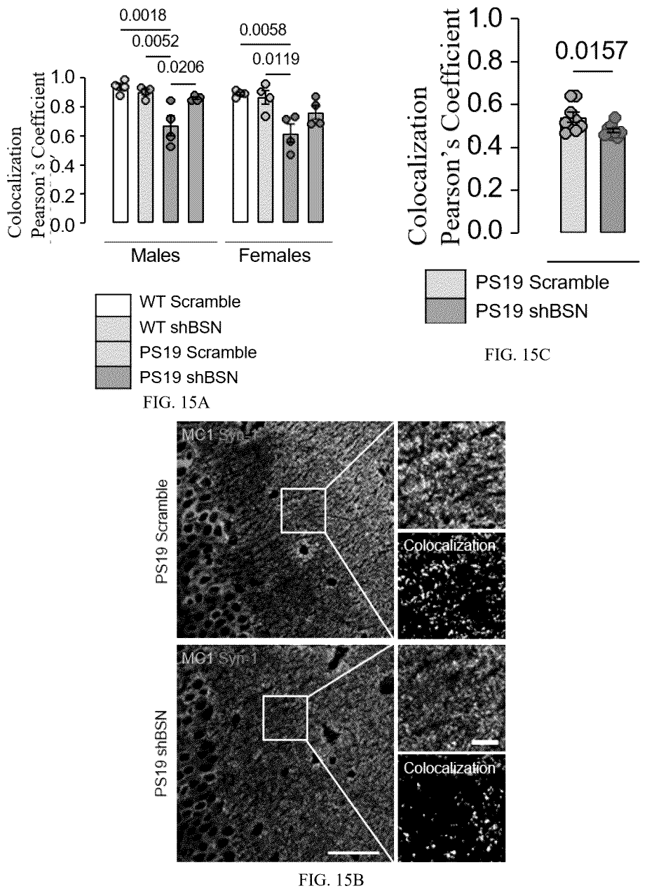

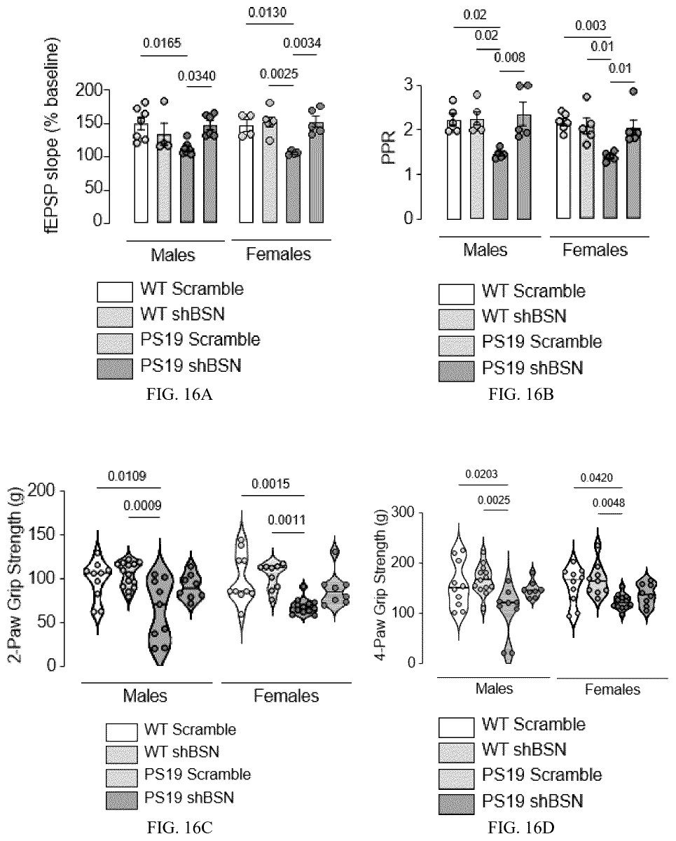

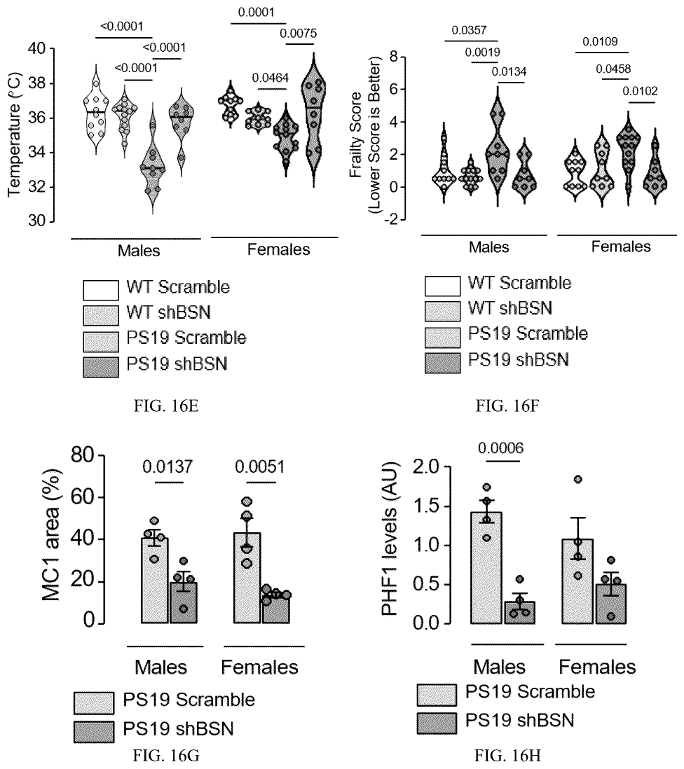

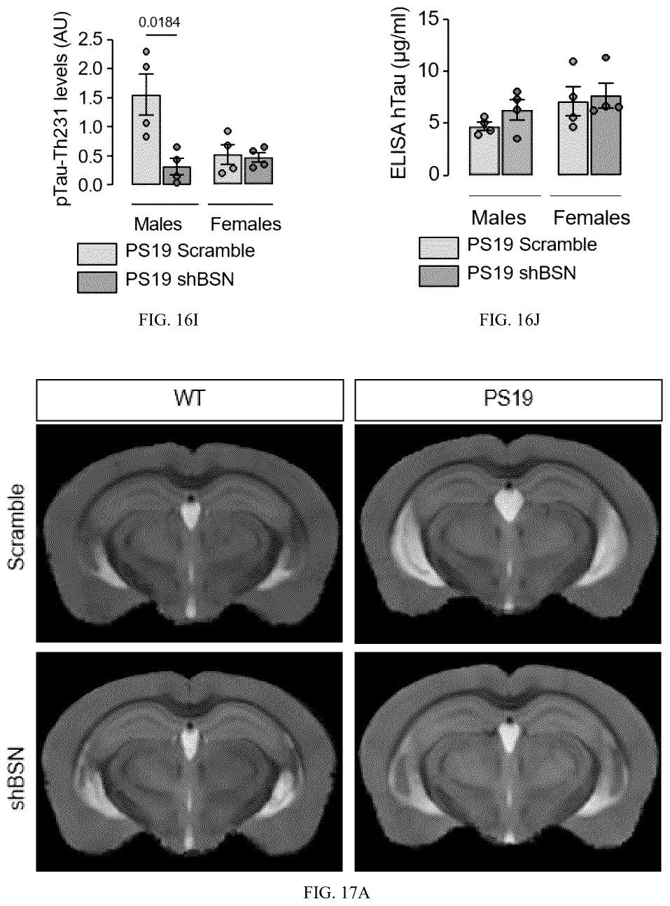

However, a decrease in BSN levels in PS19_{shBSN} mice rescued LTP (FIG. 6A-C). No difference was observed in LTP between the PS19_{shBSN} mice and the WT_{scramble} or WT_{shBSN} controls. The paired-pulse ratio (PPR) was also measured, which decreased in PS19_{scramble} vs. WT_{scramble} and WT_{shBSN} animals, reflecting diminished synaptic vesicular release probability. This reduction was rescued in PS19_{shBSN} (FIG. 6D). The beneficial effect of BSN downregulation on LTP and PPR was observed in both female and male PS19 mice (FIG. 16A-B).

FIG. 16A-J show that BSN downregulation improves behavioral and electrophysiological impairments in PS19 male and females. FIG. 16A, B: Average of fEPSP slope from minute 60 to 70 by sex (FIG. 16A) and paired-pulse ratio (PPR) (FIG. 16B) in 6-month-old WT and PS19 mice injected with shBSN or scramble shRNA. FIG. 16C-F: 2-paw test (FIG. 16C), 4-paw test (FIG. 16D), body temperature (FIG. 16E) and frailty test (FIG. 16F) in 9-month-old WT and PS19 mice injected with shBSN or scramble shRNA. FIG. 16G-J: Quantification of MC1 immunostaining as percentage of area (FIG. 16G), pTauS396/5404 (PHF1) protein levels (FIG. 16H), pTau Thr231 protein levels (FIG. 16I), and total human tau levels by ELISA (FIG. 16J) in shBSN and scrambled PS19 male and female mouse brain lysates. Data represent the mean±s.e.m. Experiments were performed with n=5 for males and n=6 for females for WT_{scramble}, n=5 for males and n=5 for females for PS19_{scramble}, n=5 for males and n=5 for females for WT_{shBSN}, and n=5 for males and n=6 for females for PS19_{shBSN} mice (FIG. 16A-B), n=10 for males and n=10 for females for WT_{scramble}, n=9 for males and n=12 for females for PS19_{scramble}, n=13 for males and n=9 for females for WT_{shBSN}, and n=8 for males and n=8 for females for PS19_{shBSN} mice (FIG. 16C-F), and n=4 per sex (FIG. 16G-J). Significance was determined by one-way ANOVA (FIG. 16A-F) and unpaired Student's t-test (FIG. 16G-J)

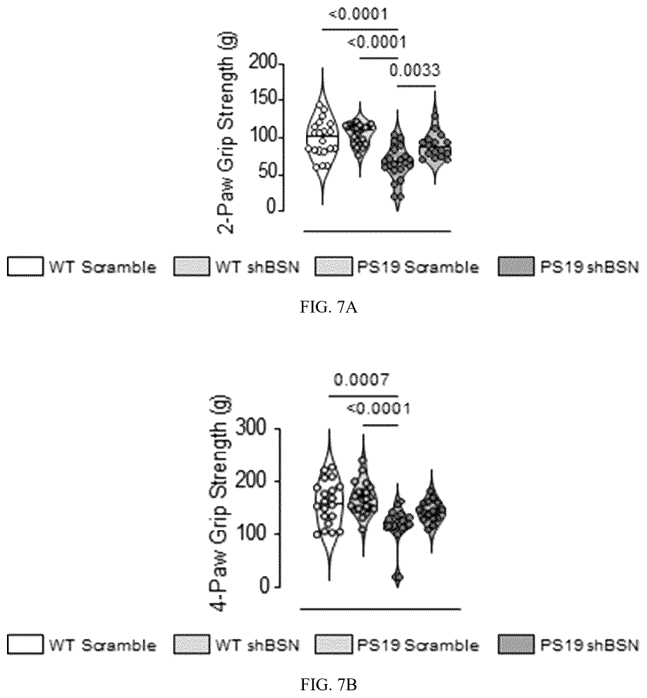

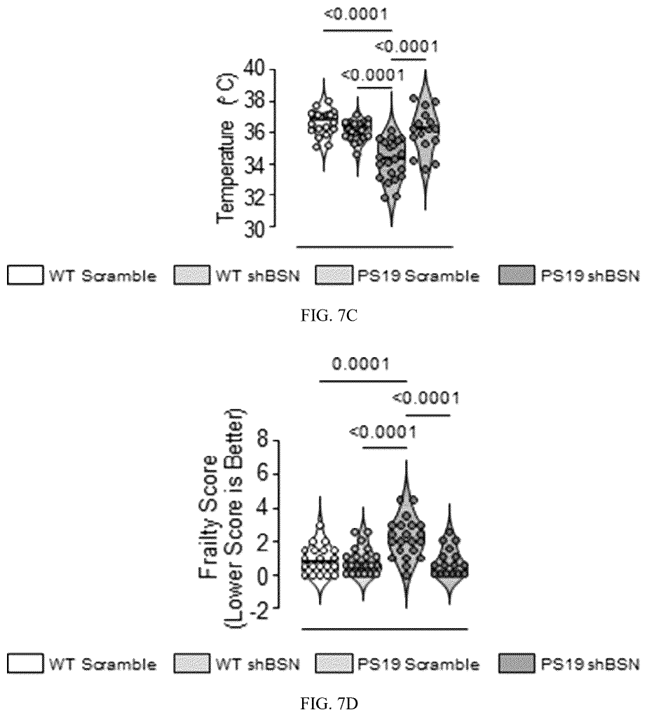

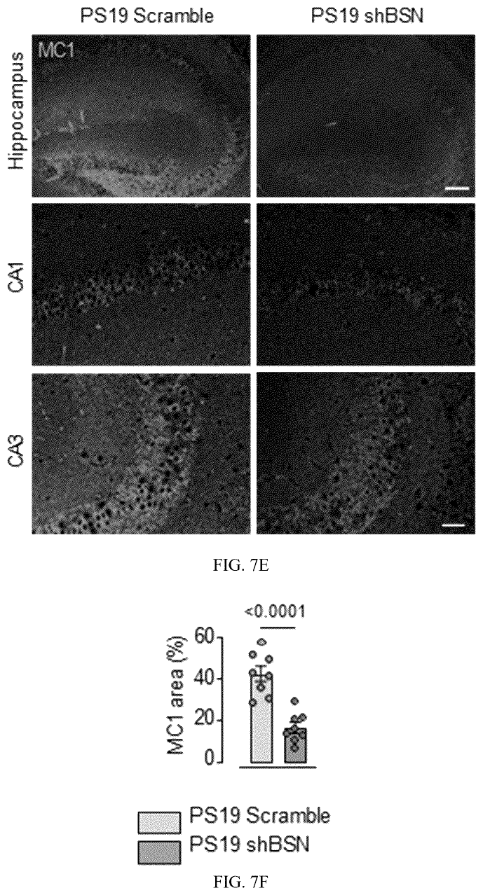

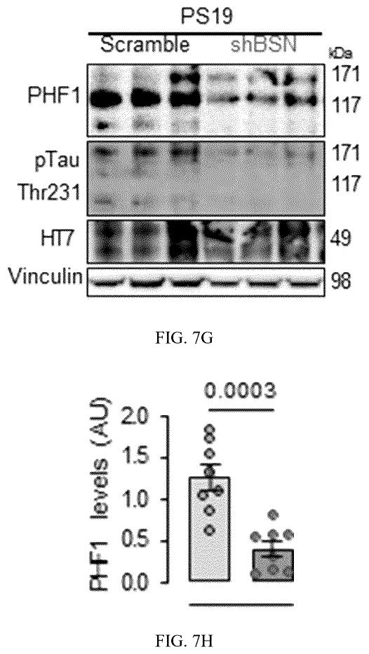

A third cohort of 9-month-old mice was subjected to behavioral and physiological tests to further assess the functional benefit of BSN downregulation. As we have recently demonstrated a decrease in motor strength in PS19 mice, (see Patel et al., “Pathological tau and reactive astrogliosis are associated with distinct functional deficits in a mouse model of tauopathy,” (2022) Neurobiol Aging 109: pp. 52-63) the grip strength of the front two paws and all four paws were tested. PS19_{scramble} mice exhibited reduced 2-paw and 4-paw grip strength compared to WT_{scramble} and WT_{shBSN} mice (FIG. 7A-B).

FIG. 7A-J shows that reducing bassoon levels improves behavioral deficit and diminishes pathological tau species in a late stage of pathology. FIG. 7A-D, 2-paw (FIG. 7A), 4-paw test (FIG. 7B), body temperature (FIG. 7C) and (FIG. 7D) frailty test in 9-month-old WT and PS19 mice injected with shBSN or scramble shRNA. FIG. 7E: Hippocampal MC1 immunostaining in 9-month-old PS19 mice cohort injected with shBSN and scramble shRNA. Scale bar: 200 μm, and 50 μm for CA1 and CA3 insets. FIG. 7F: Quantification of MC1 immunostaining in shBSN and scramble PS19 mice as percentage of area. FIG. 7G-I: Western blot and (FIG. 7G) quantification of specific antibodies against pTauS396/5404 (PHF1) (FIG. 7H) and pTau Thr231 (FIG. 7I) in shBSN and scramble PS19 mouse brain lysates. FIG. 7J: Total human tau levels by ELISA in shBSN and scrambled PS19 mouse brain lysates. Experiments were performed with n=20 for WT_{scramble}, n=21 for PS19_{scramble}, n=22 for WT_{shBSN}, and n=16 for PS19_{shBSN} mice (FIG. 7A-D), n=8 (FIG. 7E-J). Significance was determined by one-way ANOVA (FIG. 7A-D) and unpaired Student's t-test (FIG. 7F, H-J).

There were no differences between PS19_{shBSN} mice and WT_{scramble} or WT_{shBSN} mice, suggesting that BSN downregulation rescues motor impairment. The physiological characteristics of these mice were evaluated by measuring the basal core body temperature and frailty markers. PS19_{scramble} mice had lower basal body temperatures than those of the WT_{scramble} and WT_{shBSN} mice; however, downregulation of BSN levels completely rescued the decreased body temperature in PS19_{shBSN} mice (FIG. 7C).

The inventors then performed a clinical exam to assess the 26 frailty parameters. See Patel et al. 2022. PS19_{scramble} mice exhibited more frailty markers than WT_{scramble} and WT_{shBSN} mice (FIG. 7D), indicating a decline in their general health. No differences were observed between PS19_{shBsn} mice and either WT group, indicating the benefit of BSN downregulation in the context of tau pathology. Motor and physiological rescue by BSN downregulation at 9 months of age were observed in both male and female PS19 mice (FIG. 16C-F).

The inventors next tested whether BSN downregulation could reduce neurodegeneration in PS19 mice. As rising ventricular volumes and a decline in hippocampal volume indicate brain atrophy in 9-month-old PS19 mice (Wu et al., “Complement C3 Is Activated in Human AD Brain and is Required for Neurodegeneration in Mouse Models of Amyloidosis and Tauopathy,” (2019) Cell Rep 28: pp. 2111-2123), the inventors next determined the effect of BSN downregulation on ventricular and hippocampal volume by volumetric MRI (vMRI) analysis.

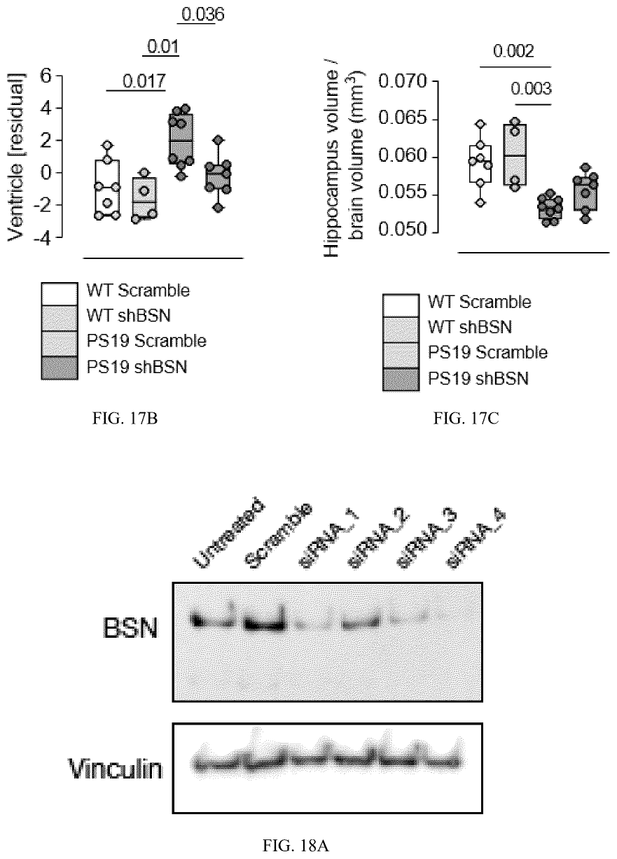

FIG. 17A-C depicts the downregulation of bassoon corresponds to reduced ventricular volume and an increment of hippocampal volume in PS19 mice. FIG. 17A-C: In vivo Magnetic resonance imaging (MRI) (FIG. 17A), quantification of ventricular (FIG. 17B), and hippocampal volume normalized to total brain volume (FIG. 17C) in 9-month-old WT and PS19 mice injected with shBSN or scramble shRNA. Data represent the mean±s.e.m. Experiments were performed with n=7 for WT_{scramble}, n=8 for PS19_{scramble}, n=4 for WT_{shBSN}, and n=7 for PS19_{shBSN} mice. Significance was determined by one-way ANOVA.

BSN downregulation did not significantly alter the ventricular nor hippocampal volume in wild-type mice (FIG. 17A-C). However, decreasing BSN levels significantly ameliorated the increase in ventricular volume and decline in hippocampal volume in PS19 mice at 9 months (FIG. 17A-C), suggesting that BSN downregulation reduces neurodegeneration in tauopathies. Due to the limited number of mice per group analyzed by vMRI, it was not possible to determine sex differences in the beneficial effects of BSN downregulation on restoring ventricular and hippocampal volume.

Finally, it was evaluated whether the functional improvement and rescue of brain atrophy observed in 9-month-old PS19 mice were associated with decreased pathological tau burden. Immunofluorescent analysis revealed that BSN downregulation reduced tau pathology in the hippocampus of 9-month-old PS19 mice (FIG. 7E-F). By western blotting, it was also confirmed that downregulation of BSN levels decreased phosphorylated tau aggregates (FIG. 7G-I) but did not affect total tau levels measured by ELISA in brain samples from 9-month-old PS19 mice (FIG. 7K). The decrease in pathological tau burden was significantly observed in 9-month-old female and male PS19 mice (FIG. 16G-J), suggesting that the effect of BSN downregulation on early stages of tau aggregation (FIG. 5A-O) has lasting effects on tau burden when higher levels of pathology have developed.

### Example 7

### Inhibition of BSN Via RNAi

The inventors contemplate that BSN can also be inhibited via administration of one or more siRNA's utilizing RNAi techniques.

In some embodiments a small interfering RNA (siRNA) is designed to target and degrade a nucleic acid encoding BSN. siRNAs are double-stranded RNA molecules of approximately 20-25 nucleotides in length. While not limited in their features, typically an siRNA is about 20 nucleotides long and has 2-nt 3′ overhangs on both ends. Each strand has a 5′ phosphate group and a 3′ hydroxyl group. In vivo, this structure is the result of processing by Dicer, an enzyme that converts either long dsRNAs or small hairpin RNAs (shRNAs) into siRNAs. However, siRNAs can also be synthesized and exogenously introduced into cells to bring about the specific knockdown of a gene of interest. Essentially any gene of which the sequence is known can be targeted based on sequence complementarity with an appropriately tailored siRNA. For example, those of ordinary skill in the art can synthesize an siRNA (see, e.g., Elbashir, et al., Nature 411: 494 (2001); Elbashir, et al. Genes Dev 15:188 (2001); Tuschl T, et al., Genes Dev 13:3191 (1999)).

In some embodiments, RNAi is utilized to inhibit BSN. RNAi represents an evolutionarily conserved cellular defense for controlling the expression of foreign genes in most eukaryotes, including humans. RNAi is typically triggered by double-stranded RNA (dsRNA) and causes sequence-specific degradation of single-stranded target RNAs (e.g., an mRNA). The mediators of mRNA degradation are small interfering RNAs (siRNAs), which are normally produced from long dsRNA by enzymatic cleavage in the cell. siRNAs are generally approximately twenty nucleotides in length (e.g. 20-23 nucleotides in length) and have a base-paired structure characterized by two-nucleotide 3′ overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, an RNase III enzyme (e.g., Dicer) converts the longer dsRNA into 20-23 nt double-stranded siRNA fragments. In some embodiments, RNAi oligonucleotides are designed to target BSN.

Experiments were performed targeting mouse BSN utilizing at least the following siRNA sequences:

(SEQ ID NO: 3)
GCGUUAUACACAGAGUUUG
(SEQ ID NO: 4)
CGUUGAUUUAGAAGCAAUA
(SEQ ID NO: 5)
GCAGAAAGUUCCAGGAUGA
(SEQ ID NO: 6)
GGACCAACUCAAAAUGGAU.

Other experiments were performed targeting human BSN utilizing at least the following siRNA sequences:

(SEQ ID NO: 7)
GCGUCAUCCUACAGACCUU
(SEQ ID NO: 8)
GCGUUAUACACAGAGUUUG
(SEQ ID NO: 9)
CCCUCAUCCCUAAGUAUGG
(SEQ ID NO: 10)
GCAUGGAGCAAAAGAUAUC.

Efficiency of siRNA Against BSN on Downregulating BSN Levels

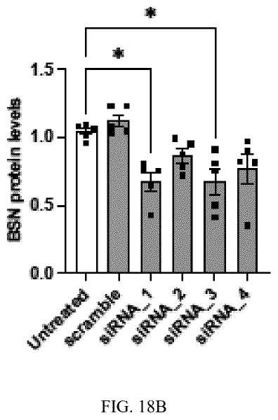

As an initial study, the inventors evaluated the efficiency of several different siRNA against BSN on downregulating BSN levels in primary neuronal culture of wild type mice. As control the inventors utilized a non-targeting siRNA scramble: UGGUUUACAUGUCGACUAA (SEQ ID NO: 11). Neurons from Wild type mice were prepared as we previously described and treated with the siRNAs against Bassoon. See Cisternas et al., “The reduction of astrocytic tau prevents amyloid-β-induced synaptotoxicity,” (2022) Brain Commun. 4(5): pp. 1-15). Fourteen days post treatment, neurons were lysed and analyzed by Western Blot. BSN was significantly downregulated in neurons treated with siRNA1 (SEQ ID NO: 3) and siRNA 3 (SEQ ID NO: 5) (FIG. 18A, B).

FIG. 18A, B depicts that siRNA against BSN downregulated BSN levels in neuronal primary cultures. FIG. 18A) Representative Western blot of cell lysate from neurons treated with siRNA anti-Bassoon. Vinculin was used as loading control. FIG. 18B) Quantification of Bassoon protein levels measure by Western blot. n=4, *p<0.05 and **p<0.01.

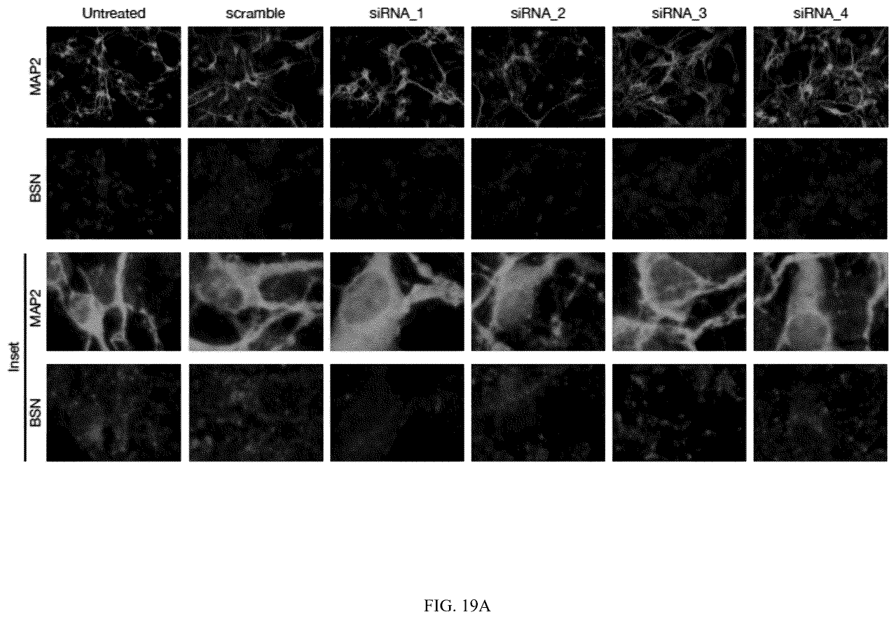

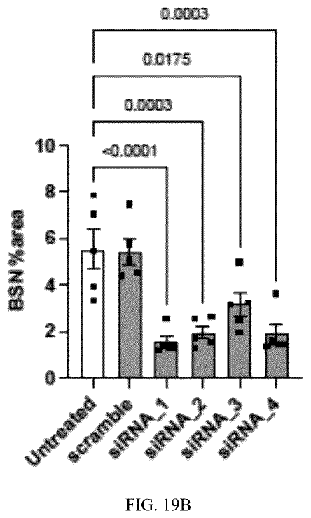

To confirm this result, in a second set of neurons treated with siRNA against BSN, the inventors performed double staining for BSN and the neuronal marker MAP2. Immunofluorescence demonstrated that all four siRNAs against Bassoon downregulated Bassoon levels without affecting neuronal integrity (FIG. 19A, B).

FIG. 19A, B shows the results of siRNA against BSN downregulated BSN levels in neuronal primary cultures. FIG. 19A) Double staining of neurons treated with siRNA against Bassoon using an anti-Bassoon antibody (red) and anti-MAP2 antibody (green). FIG. 19B) Quantification of BSN protein levels measure by percentage (%) of BSN staining per cellular area determine by MAP2 staining. n=5.

The analysis demonstrated that neurons with depleted BSN have a normal dendritic complexity, suggesting that BSN downregulation is well tolerated by neurons.

It is further contemplated that the siRNA sequences can be the administered attached to or part of any vector known in the art including plasmids, viral vectors, bacteriophages, cosmids, and artificial chromosomes.

### SUMMARY

The inventors have identified BSN as a tau-seed interactor exhibiting a preponderant role in tau-seed stability and spreading. Downregulating BSN significantly reduced tau spreading and overall tau pathology, improved synaptic integrity, rescued electrophysiological and behavioral impairments, and ameliorated brain atrophy. BSN is a large scaffolding protein (419 kDa) of the presynaptic active zone involved in the regulation of neurotransmitter release at the synapse. See Annamneedi et al., 2018. Mice with constitutive ablation of the bassoon gene show no abnormalities in brain architecture but have impaired presynaptic functions. See Altrock et al., “Functional inactivation of a fraction of excitatory synapses in mice deficient for the active zone protein bassoon,” (2003) Neuron 37: pp. 787-800. Interestingly, partial loss of Bsn (Bsn^{+/-}) causes no abnormalities, suggesting that a 50% decrease in Bsn levels is well tolerated. Altrock et al., 2003. The recently developed Bsn^{floxP/floxP} mouse model shows no neuronal differences from constitutive knockout allele by Cre-mediated recombination in the germ line and wild-type controls, suggesting that ablation of Bsn is not detrimental. BSN also regulates presynaptic ubiquitination, proteostasis, and autophage. See Okerlund et al., 2017; Waites et al., 2013, Schattleing et al. 2019. Synaptic accumulation of tau oligomers in AD is associated with dysfunction of the ubiquitin-proteasome system, suggesting that these oligomers may be an important mediator of the proteotoxicity that disrupts synapses in AD. See Tai et al., “The synaptic accumulation of hyperphosphorylated tau oligomers in Alzheimer disease is associated with dysfunction of the ubiquitin-proteasome system,” (2012) Am J Pathol 181: pp. 1426-1435.

BSN is also involved in regulating neurotransmitter release from glutamatergic synapses. See Altrock et al., 2003. The selective ablation of Bsn in excitatory neurons enhances learning performance in mice. See Anamneedi et al., 2018. These functions of BSN could affect tau pathology considering recent studies demonstrating that tau accumulation occurs predominantly in excitatory neurons and that tau induces excitotoxicity due to alterations in glutamate neurotransmission. See Fu et al., “A tau homeostasis signature is linked with the cellular and regional vulnerability of excitatory neurons to tau pathology,” (2019) Nat Neurosci 22: pp. 47-56; Hunsberger et al., “ ”P301L tau expression affects glutamate release and clearance in the hippocampal trisynaptic pathway,” (2015) J Neurochem 132: pp. 169-182; Roberson et al., “Reducing endogenous tau ameliorates amyloid beta-induced deficits in an Alzheimer's disease mouse model,” (2007) Science 316: pp. 750-754; Timmer et al., “Cerebral level of vGlut1 is increased and level of glycine is decreased in TgSwDI mice,” (2014) J Alzheimers Dis 39: pp. 89-101.

Little is known about the importance of BSN in tau pathology and neurodegeneration. The strongest evidence linking BSN with tau pathology was published in 2018, revealing four missense mutations in the BSN gene (P3866A) and aggregation of 3 and 4 repeat tau in patients with a spatial distribution of tau pathology consistent with PSP. See Yabe et al., 2018. Another study revealed increased BSN expression in patients with MSA, a neurodegenerative disease characterized by the aggregation of alpha-synuclein and tau protein. See Hashida et al., 1998; Nagaishi et al., “Tau-positive glial cytoplasmic granules in multiple system atrophy,” (2011) Neuropathology 31: pp. 299-305.

A new study recently demonstrated the toxic accumulation of BSN in the neuronal somata of mice and patients with MS. Notably, this study also demonstrated that the genetic ablation of Bsn protected mice from inflammation-induced neuroaxonal injury and enhanced neuronal survival. Overall, these studies suggest associations among BSN, neurodegenerative events, and tau pathology, and support the feasibility of BSN downregulation as a therapeutic avenue for neurodegenerative tauopathies. Shattling et al., 2019.

Apart from BSN, the inventors demonstrate that the identification of other presynaptic interactors. The association between the tau seed and presynaptic proteins is relevant considering that numerous studies have suggested that secreted tau can spread transneuronally among synaptically connected neurons and across functionally connected regions of the brain. See Wang et al., “The release and trans-synaptic transmission of Tau via exosomes,” (2017) Mol Neurodegener 12: p. 5; Vogel et al., “Spread of pathological tau proteins through communicating neurons in human Alzheimer's disease,” (2020) Nat Commun 11: p. 2612. Furthermore, depolarization-induced synaptosomes from AD brains trigger the release of tau from the presynaptic terminal, suggesting that tau secretion is regulated by presynaptic activity. See Sokolow et al., “Pre-synaptic C-terminal truncated tau is released from cortical synapses in Alzheimer's disease,” (2015) J Neurochem 133: pp. 368-379.

Tracy et al., 2022 recently demonstrated that wild-type tau interacts with numerous presynaptic proteins at the active zone in human iPSC-derived neurons, suggesting that tau is closely associated with docked vesicles at presynaptic terminals. The authors demonstrated that neuronal activity enhanced the interaction of tau with synaptic vesicle proteins, suggesting that presynaptic fusion machinery could regulate activity-dependent tau release through direct protein-protein interaction. For instance, the authors observed that tau interacts with synaptotagmin-1 (SYT1) and an increase in neuronal activity enhanced the interaction of tau with SYT1. Interestingly, we identified SYT1 as an interactor of the tau seed, supporting the notion that the tau seed interacts with presynaptic proteins strongly associated with physiological tau during neuronal activity. It is worth mentioning that in this reference, wild-type tau interactome differences were determined in relation to neuronal activity, but no studies were performed concerning aggregation or seeding activity.

In the current invention, the inventors also identified synaptogyring-3 (SYNGR3) as an interactor of the tau seed and monomeric tau. Since tau interaction with SYNGR3 inhibits presynaptic vesicle release, and SYNGR3 downregulation rescues tau-induced defects in a mouse model of tauopathy, this supports the beneficial effects of downregulating the levels of a presynaptic tau interactor.

The current disclosure reiterates the relevance of presynaptic tau interactors in disease pathogenesis via a diverse set of mechanisms that are not mutually exclusive. One mechanism could involve increased stability or resistance to degradation of a toxic tau conformer due to its interaction with presynaptic proteins. Another possibility involves the loss of physiological synaptic functions due to the interaction of these synaptic proteins with tau aggregates. A third possibility is that a tau conformer with seeding activity utilizes the presynaptic vesicle fusion machinery for neuronal release, as suggested for physiological tau, through aberrant protein interactions. In our study, we focused on the presynaptic protein bassoon as a novel interactor of the tau seed, considering the strong genetic link between BSN mutation and human tauopathies.

It has been proposed that flexible hydrophobic surfaces may provide oligomers and protofilaments the capacity to engage in aberrant interactions with metastable proteins that share distinct physicochemical properties. Proteins prone to interact with aggregates such as oligomers and protofilaments are usually larger, have a lower average hydrophobicity, and exhibit high structural flexibility; additionally, significant enrichment is observed in more intrinsically disordered regions. BSN, a large multidomain protein with long stretches of intrinsically disordered residues, is particularly prone to accumulation. The inventors hypothesize that BSN may interact with amyloid-like aggregates.

The current disclosure of cell-based results and analyses of mouse and human brain samples support the notion that BSN interacts with HMW-tau species with seeding activity but not with non-aggregated forms of tau. The fact that BSN solely or mainly interacts with a tau seed representing less than 10% of total tau in the brain could explain why BSN has not previously been identified as a tau interactor. Previous studies identified tau interactors from total cellular or brain lysate without discerning between tau aggregation state as described above which may have masked the effect of proteins that solely interact with the tau seed.

The current results indicate that BSN might contribute to tau pathogenesis by promoting the stabilization and resistance to degradation of abnormal tau structures and assemblies with strong seeding activity rather than the sole loss of its physiological functions.

In summary, the current disclosure indicates that the inhibition of the interaction so tau seed interactors, such as BSN, that could work as scaffolds or stabilizers of the pathogenic seed, present new therapeutic approach for neurodegenerative tauopathies.

As will be appreciated from the descriptions herein, a wide variety of aspects and embodiments are contemplated by the present disclosure, examples of which include, without limitation, the aspects and embodiments listed below:

The current invention provides methods using tau interactors, including BSN, to stabilize tau seeds as a therapeutic approach for neurodegenerative tauopathies including Alzheimer's disease and related disorders including chronic traumatic encephalopathy (CTE).

More specifically, the current invention provides:

Methods of treating neurodegenerative tauopathies in a patient in need thereof comprising administration of an agent that reduces or inhibits the interaction of a tau seed interactor with intracellular tau proteins.

Methods of treating neurodegenerative tauopathies in a patient in need thereof comprising administration of an agent that reduces or inhibits the interaction of a tau seed interactor with intracellular tau proteins, wherein tau seed interactor is Bassoon (BSN), a scaffolding protein of the presynaptic active zone.

Methods of treating neurodegenerative tauopathies in a patient in need thereof comprising administration of an agent that reduces or inhibits the interaction of a tau seed interactor with intracellular tau proteins, wherein, the agent comprises an adeno-associated virus (AAV)-containing an a short-hairpin RNA (shRNA) against BSN (shBSN).

Methods of treating neurodegenerative tauopathies in a patient in need thereof comprising administration of an agent that reduces or inhibits the interaction of a tau seed interactor with intracellular tau proteins, wherein the agent comprises an adeno-associated virus (AAV)-containing an a short-hairpin RNA (shRNA) against BSN (shBSN), wherein the shBSN sequence is CCTAACGCTTTCCTCTGACAT (SEQ ID NO: 1).

Methods of treating neurodegenerative tauopathies in a patient in need thereof comprising administration of an agent that reduces or inhibits the interaction of a tau seed interactor with intracellular tau proteins, wherein the agent is attached to or part of any vector known in the art including plasmids, viral vectors, bacteriophages, cosmids, and artificial chromosomes.

Methods of treating neurodegenerative tauopathies in a patient in need thereof comprising administration of an agent that reduces or inhibits the interaction of a tau seed interactor with intracellular tau proteins, wherein the agent comprises a monoclonal antibody directed against the tau seed interactor. In still other embodiments, the agent is an siRNA or antisense oligonucleotide that targets BSN.

Methods of treating neurodegenerative tauopathies in a patient in need thereof comprising administration of an agent that reduces or inhibits the interaction of a tau seed interactor with intracellular tau proteins, wherein the agent comprises a monoclonal antibody directed against BSN.

Any of the methods disclosed herein, wherein the agent that reduces or inhibits the interaction of a tau seed interactor with intracellular tau proteins leads to a reduction of neurodegeneration in a patient with a neurodegenerative tauopathies.

Any of the methods disclosed herein, wherein the agent that reduces or inhibits the interaction of a tau seed interactor with intracellular tau proteins leads to a behavioral improvement in a patient with a neurodegenerative tauopathies.

Methods of preventing neurodegenerative tauopathies in a patient in need thereof comprising administration of an agent that reduces or inhibits the interaction of a tau seed interactor with intracellular tau proteins.

Any of the methods disclosed herein, wherein the neurodegenerative tauopathies can be neurodegenerative diseases characterized by abnormal metabolism of misfolded tau proteins which in turn leads to intracellular accumulation and formation of neurofibrillary tangles (NFT). In any embodiment, the neurodegenerative tauopathies are selected from Alzheimer's disease, progressive supranuclear palsy (PSP), frontotemporal lobar degeneration (FTLD-TAU), corticobasal degeneration, Pick's disease (frontal temporal dementia), chronic traumatic encephalopathy (CTE), and primary age related taupathy.

Methods of treating or preventing Alzheimer's disease in a patient in need thereof comprising administration of an agent that reduces that reduces the interaction of a tau seed interactor with intracellular tau proteins.

Methods of treating or preventing chronic traumatic encephalopathy (CTE) in a patient in need thereof comprising administration of an agent that reduces the interaction of a tau seed interactor with intracellular tau proteins.

Pharmaceutical compositions comprising an agent that reduces the interaction of a tau seed interactor with intracellular tau proteins, wherein the pharmaceutical compositions can further comprise one or more pharmaceutically acceptable carriers, diluents or excipients.

Any of the methods disclosed herein wherein the method further comprises the administration of one or more additional therapeutic agents.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating neurodegenerative tauopathies in a patient in need thereof comprising administration of an agent that reduces or inhibits the interaction of a tau seed interactor with intracellular tau proteins.

2. The method of claim 1, wherein the tau seed interactor is Bassoon (BSN).

3. The method of any of claims 1 and 2, wherein the agent comprises a vector containing a short-hairpin RNA (shRNA) against BSN (shBSN).

4. The method of claim 3, wherein the sequence of the short-hairpin RNA (shRNA) against BSN (shBSN) is CCTAACGCTTTCCTCTGACAT (SEQ. ID. NO. 1).

5. The method of claim 3, wherein the vector is selected from the group consisting of plasmids, viral vectors, bacteriophages, cosmids, and artificial chromosomes.

6. The method of claim 5, wherein the viral vector comprises an adeno-associated virus (AAV).

7. The method of claim 1, wherein the agent comprises a monoclonal antibody directed against the tau seed interactor.

8. The method of claim 7, wherein the agent comprises a monoclonal antibody directed against BSN.

9. The method of claim 1, wherein the agent comprises an siRNA targeting the tau seed interactor.

10. The method of claim 9, wherein the agent comprises an siRNA targeting BSN.

11. The method of claim 1, wherein the agent that reduces or inhibits the interaction of a tau seed interactor with intracellular tau proteins leads to a reduction of neurodegeneration in a patient with a neurodegenerative tauopathies.

12. The method of claim 1, wherein the agent that reduces or inhibits the interaction of a tau seed interactor with intracellular tau proteins leads to a behavioral improvement in a patient with a neurodegenerative tauopathies.

13. A method of preventing neurodegenerative tauopathies in a patient in need thereof comprising administration of an agent that reduces or inhibits the interaction of a tau seed interactor with intracellular tau proteins.

14. The method of claim 13, wherein the tau seed interactor is Bassoon (BSN).

15. The method of any of claims 13 and 14, wherein the agent comprises a vector containing a short-hairpin RNA (shRNA) against BSN (shBSN).

16. The method of claim 15, wherein the sequence of the short-hairpin RNA (shRNA) against BSN (shBSN) is CCTAACGCTTTCCTCTGACAT (SEQ. ID. NO. 1).

17. The method of claim 15, wherein the vector is selected from the group consisting of plasmids, viral vectors, bacteriophages, cosmids, and artificial chromosomes.

18. The method of claim 17, wherein the viral vector comprises an adeno-associated virus (AAV).

19. The method of claim 13, wherein the agent comprises a monoclonal antibody directed against the tau seed interactor.

20. The method of claim 19, wherein the agent comprises a monoclonal antibody directed against BSN.

21. The method of claim 13, wherein the agent comprises an siRNA targeting the tau seed interactor.

22. The method of claim 21, wherein the agent comprises an siRNA targeting BSN.

23. The method of any of claims 1-22, wherein the neurodegenerative tauopathies is a selected from the group consisting of Alzheimer's disease, progressive supranuclear palsy (PSP), frontotemporal lobar degeneration (FTLD-TAU), corticobasal degeneration, Pick's disease (frontal temporal dementia), chronic traumatic encephalopathy (CTE), and primary age related taupathy.

24. A method of treating or preventing Alzheimer's disease in a patient in need thereof comprising administration of an agent that reduces the interaction of a tau seed interactor with intracellular tau proteins.

25. A method of treating or preventing chronic traumatic encephalopathy (CTE) in a patient in need thereof comprising administration of an agent that reduces the interaction of a tau seed interactor with intracellular tau proteins.

26. A pharmaceutical composition comprising an agent that reduces the interaction of a tau seed interactor with intracellular tau proteins.

27. The pharmaceutical composition of claim 25, wherein the tau seed interactor is Bassoon (BSN).

28. The pharmaceutical composition of any of claims 26 and 27, wherein the agent comprises a vector containing a short-hairpin RNA (shRNA) against BSN (shBSN).

29. The pharmaceutical composition of claim 28, wherein the sequence of the short-hairpin RNA (shRNA) against BSN (shBSN) is CCTAACGCTTTCCTCTGACAT (SEQ. ID. NO. 1).

30. The pharmaceutical composition of claim 28, wherein the vector selected from the group consisting of plasmids, viral vectors, bacteriophages, cosmids, and artificial chromosomes.

31. The pharmaceutical composition of claim 30, wherein the viral vector comprises an adeno-associated virus (AAV).

32. The pharmaceutical composition of claim 26, wherein the agent comprises a monoclonal antibody directed against the tau seed interactor.

33. The pharmaceutical composition of claim 32, wherein the agent comprises a monoclonal antibody directed against BSN.

34. The pharmaceutical composition of claim 26, wherein the agent comprises an siRNA targeting the tau seed interactor.

35. The pharmaceutical composition of claim 34, wherein the agent comprises an siRNA targeting BSN.

36. The pharmaceutical composition of any of claims 26-35, wherein the pharmaceutical composition is administered to a patient in need thereof for the treatment or prevention of neurodegenerative tauopathies.

37. The pharmaceutical composition of claim 36, wherein the neurodegenerative tauopathies is a selected from the group consisting of Alzheimer's disease, progressive supranuclear palsy (PSP), frontotemporal lobar degeneration (FTLD-TAU), corticobasal degeneration, Pick's disease (frontal temporal dementia), chronic traumatic encephalopathy (CTE), and primary age related taupathy.

38. The pharmaceutical composition of claim 37, wherein the neurodegenerative tauopathy is Alzheimer's disease.

39. The pharmaceutical composition of claim 37, wherein the neurodegenerative tauopathy is chronic traumatic encephalopathy (CTE).

40. The pharmaceutical composition of any of claims 26-39, wherein the pharmaceutical composition can further comprise one or more pharmaceutically acceptable carriers, diluents or excipients and optionally one or more additional therapeutic agents.

41. The methods of any of claims 1-25, wherein the method further comprises the administration of one or more additional therapeutic agents.

42. The method of claim 10, wherein the siRNA targeting BSN comprises one or more siRNA comprises one or more of SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.


SEQUENCE LISTING (11 sequences)

SEQ ID NO: 1 - RNA, 21 nt, synthetic construct
  source 1..21: mol_type = other RNA
cctaacgctttcctctgacat

SEQ ID NO: 2 - RNA, 21 nt, synthetic construct
  source 1..21: mol_type = other RNA
cctaaggttaagtcgccctcg

SEQ ID NO: 3 - RNA, 19 nt, synthetic construct
  source 1..19: mol_type = other RNA
gcgttatacacagagtttg

SEQ ID NO: 4 - RNA, 19 nt, synthetic construct
  source 1..19: mol_type = other RNA
cgttgatttagaagcaata

SEQ ID NO: 5 - RNA, 19 nt, synthetic construct
  source 1..19: mol_type = other RNA
gcagaaagttccaggatga

SEQ ID NO: 6 - RNA, 19 nt, synthetic construct
  source 1..19: mol_type = other RNA
ggaccaactcaaaatggat

SEQ ID NO: 7 - RNA, 19 nt, synthetic construct
  source 1..19: mol_type = other RNA
gcgtcatcctacagacctt

SEQ ID NO: 8 - RNA, 19 nt, synthetic construct
  source 1..19: mol_type = other RNA
gcgttatacacagagtttg

SEQ ID NO: 9 - RNA, 19 nt, synthetic construct
  source 1..19: mol_type = other RNA
ccctcatccctaagtatgg

SEQ ID NO: 10 - RNA, 19 nt, synthetic construct
  source 1..19: mol_type = other RNA
gcatggagcaaaagatatc

SEQ ID NO: 11 - RNA, 19 nt, synthetic construct
  source 1..19: mol_type = other RNA
tggtttacatgtcgactaa